(19) United States
(12) Patent Application Publication
(10) Pub. No.: US 2021/0222253 A1
(43) Pub. Date: Jul. 22, 2021

(54) IDENTIFICATION OF BIOMARKERS OF GLIOBLASTOMA AND METHODS OF USING THE SAME

(71) Applicant: 10x Genomics, Inc., Pleasanton, CA (US)

(72) Inventor: Cedric Uytingco, Pleasanton, CA (US)

(21) Appl. No.: 17/153,681

(22) Filed: Jan. 20, 2021

Related U.S. Application Data
(60) Provisional application No. 63/114,404, filed on Nov. 16, 2020.
(60) Provisional application No. 63/108,273, filed on Oct. 30, 2020.
(60) Provisional application No. 62/964,063, filed on Jan. 21, 2020.

(51) Int. Cl. C12Q 1/6886; G01N 33/574

(52) U.S. Cl. CPC C12Q 1/6886; G01N 33/57407; C12Q 2600/106; G01N 2800/52; C12Q 2600/158

(57) ABSTRACT

Provided herein are methods of detecting biomarkers and/or candidate biomarkers for glioblastoma and uses of the same.

### CROSS-REFERENCE TO RELATED APPLICATIONS

This application claims priority and benefit from U.S. Provisional Patent Application 62/964,063, filed Jan. 21, 2020; U.S. Provisional Patent Application 63/108,273, filed Oct. 30, 2020; and U.S. Provisional Patent Application 63/114,404 filed Nov. 16, 2020, the contents and disclosures of which are incorporated herein by reference in their entireties.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provide a lot of analyte data for single cells, but fail to provide information regarding the position of the single cell in a parent biological sample (e.g., tissue sample).

Glioblastoma is a common type of malignant brain tumor with a median survival time of 12-14 months. Aside from standard histological assessment of these tumors, RNA sequencing from these diseased tissues can provide insights in gene expression from biomarkers, which may help support a pathologist's interpretations that could dictate the clinical outcome. However, standard RNA sequencing workflows require dissociation of the tissue, resulting in the loss of spatial patterns of gene expression.

Genetic material, and related gene and protein expression, influences cellular fate and behavior. The spatial heterogeneity in developing systems has typically been studied via RNA hybridization, immunohistochemistry, fluorescent reporters, or purification or induction of pre-defined subpopulations and subsequent genomic profiling (e.g., RNA-seq). Such approaches, however, rely on a small set of pre-defined markers, therefore introducing selection bias that limits discovery and making it costly and laborious to localize RNA transcriptome-wide.

### SUMMARY

Provided herein are methods of differentiating cell types in a biological sample comprising: (a) contacting the biological sample with a plurality of capture probes, wherein a capture probe comprises a capture domain and a spatial barcode having a sequence; (b) releasing nucleic acids from the biological sample, wherein members of the released nucleic acids are specifically bound by the capture domain(s); (c) determining, for the nucleic acids that are specifically bound by the capture domain(s), (1) all or a portion of a sequence of the spatial barcode, or a complement thereof, and (2) all or a portion of a sequence of the nucleic acid, or a complement thereof, and using the determined sequences of (1) and (2) to identify the location and amount of the nucleic acids in the biological sample; (d) comparing the determined location and amount of the nucleic acids at a plurality of different locations in the biological sample; and (e) sorting a subset of the nucleic acids of (d) into a cluster based on the location and amount of the nucleic acids at the plurality of different locations in the biological sample, and using the cluster(s) to differentiate cell types in the biological sample.

Also provided herein are methods of generating an image of a biological sample comprising: (a) contacting the biological sample with a plurality of capture probes, wherein a capture probe comprises a capture domain and a spatial barcode having a sequence; (b) releasing nucleic acids from the biological sample, wherein members of the released nucleic acids are specifically bound by the capture domain(s); (c) determining, for the nucleic acids that are specifically bound by the capture domain(s), (1) all or a portion of a sequence of the spatial barcode, or a complement thereof, and (2) all or a portion of a sequence of the nucleic acid, or a complement thereof, and using the determined sequences of (1) and (2) to identify the location and amount of the nucleic acids in the biological sample; (d) comparing the determined location and amount of the nucleic acids at a plurality of different locations in the biological sample; and (e) sorting a subset of the nucleic acids of (d) into a cluster based on the determined location and amount of the nucleic acids at the plurality of different locations in the biological sample, and using the cluster(s) to biological sample to generate an image of the biological sample. Also provided herein are method of detecting molecular heterogeneity in a biological sample comprising: (a) contacting a biological sample from the subject with a plurality of capture probes, wherein a capture probe comprises a capture domain and a spatial barcode having a sequence; (b) releasing nucleic acids from the biological sample, wherein members of the released nucleic acids are specifically bound by the capture domain(s); (c) determining, for the nucleic acids that are specifically bound by the capture domain(s), (1) all or a portion of a sequence of the spatial barcode or a complement thereof, and (2) all or a portion of a sequence of the nucleic acid or a complement thereof, and using the determined sequences of (1) and (2) to identify the location and amount of the nucleic acids in the biological sample; (d) comparing the determined location and amount of the nucleic acids at a plurality of different locations in the biological sample; and (e) sorting a subset of the nucleic acids of (d) into a cluster based on the determined location and amount of the nucleic acids at the plurality of different locations in the biological sample, and using the cluster(s) to identify molecular heterogeneity in the biological sample relative to a reference biological sample.

Also provided herein are methods of identifying a subject as having abnormal gene expression in at least one tissue comprising: (a) contacting a biological sample obtained from the subject with a plurality of capture probes, wherein a capture probe comprises a capture domain and a spatial barcode having a sequence; (b) releasing nucleic acids from the biological sample, wherein members of the released nucleic acids are specifically bound by the capture domain(s); (c) determining, for the nucleic acids that are specifically bound by the capture domain(s) (1) all or a portion of a sequence of the spatial barcode or a complement thereof, and (2) all or a portion of a sequence of the nucleic acid or a complement thereof, and using the determined sequences of (1) and (2) to identify the location and amount of the nucleic acids in the biological sample; (d) comparing the determined location and amount of the nucleic acids at a plurality of different locations in the biological sample; and (e) sorting a subset of the nucleic acids of (d) into a cluster based on the determined location and amount of the nucleic acids at the plurality of different locations in the biological sample, and using the cluster(s) to identify at least one region in the biological sample with abnormal gene expression relative to a reference biological sample. In some embodiments, the amount of one or more nucleic acids falls outside a predetermined threshold.

Also provided herein are methods of identifying a subject as having a cellular anomaly comprising: (a) contacting a biological sample from the subject with a plurality of capture probes, wherein a capture probe comprises a capture domain and a spatial barcode having a sequence; (b) releasing nucleic acids from the biological sample, wherein members of the released nucleic acids are specifically bound by the capture domain(s); (c) determining, for the nucleic acids that are specifically bound by the capture domain(s) (1) all or a portion of a sequence of the spatial barcode, or a complement thereof, and (2) all or a portion of a sequence of the nucleic acid, or a complement thereof, and using the determined sequences of (1) and (2) to identify the location and amount of the nucleic acids in the biological sample; (d) comparing the determined location and amount of the nucleic acids at a plurality of different locations in the biological sample; and (e) sorting a subset of the nucleic acids of (d) into a cluster based on the determined location and amount of the nucleic acids at the plurality of different locations in the biological sample, and using the cluster(s) to identify at least one cellular anomaly in the biological sample.

Also provided herein are methods of assessing the efficacy of a treatment or therapy in a subject comprising: (a) contacting a biological sample from the subject with a plurality of capture probes, wherein a capture probe comprises a capture domain and a spatial barcode having a sequence; (b) releasing nucleic acids from the biological sample, wherein members of the released nucleic acids are specifically bound by the capture domain(s); (c) determining, for the nucleic acids that are specifically bound by the capture domain(s) (1) all or a portion of a sequence of the spatial barcode, or a complement thereof, and (2) all or a portion of a sequence of the nucleic acid, or a complement thereof, and using the determined sequences of (1) and (2) to identify the location and amount of the nucleic acids in the biological sample; (d) comparing the determined location and amount of the nucleic acids at a plurality of different locations in the biological sample; and (e) sorting a subset of the nucleic acids of (d) into a cluster based on the determined location and amount of the nucleic acids at the plurality of different locations in the biological sample, and using the cluster(s) to identify at least one region in the biological sample having restored gene expression.

Also provided herein are methods of comparing at least two biological samples comprising: (a) contacting the first biological sample with a plurality of capture probes, wherein a capture probe comprises a capture domain and a spatial barcode having a sequence; (b) releasing nucleic acids from the biological sample, wherein members of the released nucleic acids are specifically bound by the capture domain(s); (c) determining, for the nucleic acids that are specifically bound by the capture domain(s) (1) all or a portion of a sequence of the spatial barcode, or a complement thereof, and (2) all or a portion of a sequence of the nucleic acid, or a complement thereof, and using the determined sequences of (1) and (2) to identify the location and amount of the nucleic acids in the biological sample; (d) comparing the determined location and amount of nucleic acids at a plurality of different locations in the biological sample; (e) sorting a subset of the nucleic acids of (d) into a first set of clusters based on the determined location and amount of the nucleic acids at the plurality of different locations in the biological sample, and using the clusters to differentiate cell types in the biological sample; (f) performing steps (a) to (e) on a second biological sample to identify a second set of clusters; and (g) comparing the two sets of clusters.

In some embodiments, the first biological sample is from the same subject as the second biological sample. In some embodiments, there is a period of time between acquiring the first biological sample and acquiring the second biological or subsequent samples from the subject. In some embodiments, the period of time is about 1 month to about two years. In some embodiments, the period of time is about 1 year. In some embodiments, the method further comprises comparing the clusters from additional biological samples obtained from the subject before and after the period of time.

In some embodiments, the first biological sample is obtained from a first subject and the second biological sample is obtained from a second subject. In some embodiments, the second biological sample is obtained from a healthy subject. In some embodiments, the first biological sample is obtained from a subject at risk (e.g., increased risk) of developing a disease.

Also provided herein are methods that include: (a) contacting a biological sample obtained from a subject with a plurality of capture probes, wherein a capture probe comprises a capture domain and a spatial barcode having a sequence; (b) releasing nucleic acids from the biological sample, wherein members of the released nucleic acids are specifically bound by the capture domain(s); (c) determining, for the nucleic acids that are specifically bound by the capture domain(s) (1) all or a portion of a sequence of the spatial barcode or a complement thereof, and (2) all or a portion of a sequence of the nucleic acid or a complement thereof, and using the determined sequences of (1) and (2) to identify the location and amount of the nucleic acids in the biological sample; (d) comparing the determined location and amount of nucleic acids at a plurality of different locations in the biological sample; (e) sorting a subset of the nucleic acids of (d) into a set of clusters based on the determined location and amount of nucleic acids at the plurality of different locations in the biological sample, and using the clusters to differentiate cell types in the biological sample; and (f) comparing the set of clusters to a reference set of clusters.

In some embodiments, the reference set of clusters is a normalized set of clusters from more than one reference biological sample. In some embodiments, each of the more than one reference biological sample includes the same type of tissue as the biological sample obtained from the subject.

In some embodiments, the cluster is identified using nonlinear dimensionality reduction. In some embodiments, the cluster is identified using t-distributed stochastic neighbor embedding (t-SNE). In some embodiments, the cluster is identified using global t-distributed stochastic neighbor embedding (g-SNE). In some embodiments, the cluster is identified using parametric t-SNE. In some embodiments, the cluster is identified using hierarchical t-SNE. In some embodiments, the cluster is identified using uniform manifold approximation and projection (UMAP).

In some embodiments, 2 to 200 clusters are identified. In some embodiments, 2 to 10 clusters are identified. In some embodiments, a cluster consists of about 2 to about 25,000 genes.

In some embodiments, the method further comprises identifying a subpopulation of cells in the biological sample. In some embodiments, the biological sample comprises epithelial tissue, a connective tissue, a muscle tissue, an adipose tissue, a nervous tissue, an embryonic tissue, or a combination thereof.

In some embodiments, the biological sample comprises brain tissue, a spinal cord tissue, a skin tissue, an adipose tissue, an intestinal tissue, a colon tissue, a cervical tissue, a vaginal tissue, a muscle tissue, a cardiac tissue, a liver tissue, a pancreatic tissue, a kidney tissue, a spleen tissue, a lymph node tissue, a bone marrow tissue, a cartilage tissue, a retinal tissue, a corneal tissue, a breast tissue, a prostate tissue, a bladder tissue, a tracheal tissue, a lung tissue, a uterine tissue, a stomach tissue, a thyroid tissue, a thymus tissue, or a combination thereof.

In some embodiments, the biological sample is obtained from a biopsy. In some embodiments, the biological sample is obtained from a surgical excision. In some embodiments, the biological sample was collected during an endoscopy or colposcopy.

In some embodiments, the biological sample is a frozen tissue sample. In some embodiments, the biological sample is a formalin-fixed, paraffin-embedded (FFPE) sample. In some embodiments, the nucleic acid is DNA. In some embodiments, the Dais genomic DNA. In some embodiments, the DNA is mitochondrial DNA. In some embodiments, the nucleic acid is RNA. In some embodiments, the RNA is mRNA.

Also disclosed herein is a method of diagnosing a subject as having glioblastoma, wherein the method comprises: (a) determining a level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, or a byproduct or precursor or degradation product thereof, in a biological sample from a subject; and (b) identifying a subject having an elevated level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, in the biological sample as compared to a reference level, as having glioblastoma.

Also disclosed herein is a method of diagnosing a subject as having glioblastoma, wherein the method comprises: (a) determining a level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, CD44, POSTN, NES, TERT, UMOD, SGK1, GPR37L1, ISG15, or RGS5, or a byproduct or precursor or degradation product thereof, in a biological sample from a subject; and (b) identifying a subject having an elevated level of COL1A1, COL3A1, COL8A1, WEE1, CHI3L1, MGP, SRPX, SERPINE1, COL1A2, TIMIP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, CD44, POSTN, NES, TERT, UMOD, SGK1, GPR37L1, ISG15, or RGS5, in the biological sample as compared to a reference level, as having glioblastoma.

In some instances, the methods further comprise (c) determining a level of SPOCD1, DDK1, TNC, GBE1, SMIM3, CLIC1, MT1X, and CYR61, or a byproduct or precursor or degradation product thereof, in a biological sample from a subject; and (d) identifying a subject having an elevated level of SPOCD1, DDK1, TNC, GBE1, SMIM3, CLIC1, MT1X, and CYR61, in the biological sample as compared to a reference level, as having glioblastoma.

Also disclosed herein is a method of diagnosing a subject as having glioblastoma, wherein the method comprises: (a) determining a level of SPOCD1, DDK1, TNC, GBE1, SMIM3, CLIC1, MTX, and CYR61, or a byproduct or precursor or degradation product thereof, in a biological sample from a subject; and (b) identifying a subject having an elevated level of SPOCD1, DDK1, TNC, GBE1, SMIM3, CLIC1, MT1X, and CYR61, in the biological sample as compared to a reference level, as having glioblastoma.

Also disclosed herein is a method of diagnosing a subject as having glioblastoma, wherein the method comprises: (a) determining elevated abundance of ionized calcium-binding adaptor molecule 1 (IBA1); (b) determining a level of one or more biomarkers selected from DKK1, CHI3L1, HS2ST1, EGR1, TCIM, PLIN2, APOC1, FOS, MGP, SPP1, RPL17, TNC, IFITM3, MT2A, TMSB4X, TMSB10, PDPN, COX6C, VIM, CLIC1, IFITM2, TCEAL9, RPL12, TAGLN, and NAMPT, or a byproduct or precursor or degradation product thereof, in areas of a biological sample from a subject having elevated IBA1 compared to a reference level; and (c) identifying a subject having an elevated level the one or more biomarkers in the areas as compared to the reference level, as having glioblastoma. In some instances, the one or more biomarkers are selected from the group consisting of DKK1, HS2ST1, EGR1, TCIM, FOS, RPL17, TNC, IFITM3, TMSB4X, TMSB10, COX6C, CLIC1, TCEAL9, and RPL12.

Also disclosed herein is a method of diagnosing a subject as having glioblastoma, wherein the method comprises: (a) determining a level of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product thereof, in a biological sample from a subject; and (b) identifying a subject having a decreased level of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, and MT-ND5, or a byproduct or precursor or degradation product thereof, in the biological sample as compared to a reference level, as having glioblastoma.

In some instances, the methods further comprise (c) determining a level of NAPB, BASP1, RUNDC3A, NEFM, RAB3A, GNG3, KIF1A, ATP1A3, CNTN1, CELF4, SYN2, TUBB4A, GRIN1, or a byproduct or precursor or degradation product thereof, in a biological sample from a subject; and (d) identifying a subject having a decreased level of NAPB, BASP1, RUNDC3A, NEFM, RAB3A, GNG3, KIF1A, ATP1A3, CNTN1, CELF4, SYN2, TUBB4A, GRIN1, or a byproduct or precursor or degradation product thereof, in the biological sample as compared to a reference level, as having glioblastoma.

Also disclosed herein is a method of diagnosing a subject as having glioblastoma, wherein the method comprises: (a) determining a level of NAPB, BASP1, RUNDC3A, NEFM, RAB3A, GNG3, KIF1A, ATP1A3, CNTN1, CELF4, SYN2, TUBB4A, GRIN1, or a byproduct or precursor or degradation product thereof, in a biological sample from a subject; and (b) identifying a subject having a decreased level of NAPB, BASP1, RUNDC3A, NEFM, RAB3A, GNG3, KIF1A, ATP1A3, CNTN1, CELF4, SYN2, TUBB4A, GRIN1, or a byproduct or precursor or degradation product thereof, in the biological sample as compared to a reference level, as having glioblastoma.

Also disclosed herein is a method of diagnosing a subject as having glioblastoma, wherein the method comprises: (a) determining elevated abundance of ionized calcium-binding adaptor molecule 1 (IBA1); (b) determining a level of one or more biomarkers selected from HBA2, HBB, HBA1, COL1A2, MALAT1, RBM25, SLC25A37, NKTR, LUC7L3, ATP1A2, PNISR, MEG3, IFI44L, FAM133B, PNN, PLEKHA4, PTMS, BDP1, MTRNR2L12, SREK1, ARGLU1, XAF1, MTRNR2L8, SRRM2, and COL4A1, or a byproduct or precursor or degradation product thereof, in areas of a biological sample from a subject having elevated IBA1 compared to a reference level; and (c) identifying a subject having an decreased level of the one or more biomarkers in the areas as compared to the reference level, as having glioblastoma.

In some instances, the one or more biomarkers is selected from the group consisting of HBA2, HBB, HBA1, MALAT1, RBM25, SLC25A37, NKTR, LUC7L3, PNISR, MEG3, IFI44L, FAM133B, PNN, PLEKHA4, PTMS, BDP1, MTRNR2L12, SREK1, ARGLU1, XAF1, MTRNR2L8, and SRRM2. In some instances, the method further comprises confirming a diagnosis of glioblastoma in the subject by obtaining an image of the subject's brain or performing neurological testing on the subject. In some instances, the method further comprises administering a treatment of glioblastoma to the subject.

Also disclosed herein is a method of identifying a subject as having an increased likelihood of developing glioblastoma, wherein the method comprises: (a) determining a level of COL1A1, COL3A1, COL8A1, WEE1, CHI3L1, MGP, SRPX, SERPINE1, COL1A2, TIMIP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, or a byproduct or precursor or degradation product thereof, in a biological sample from a subject; and (b) identifying a subject having an elevated level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, or a byproduct or precursor or degradation product thereof, in the biological sample as compared to a reference level, as having an increased likelihood of developing glioblastoma.

Also disclosed herein is a method of identifying a subject as having an increased likelihood of developing glioblastoma, wherein the method comprises: (a) determining a level of CD44, POSTN, NES, TERT, UMOD, SGK1, GPR37L1, ISG15, or RGS5, or a byproduct or precursor or degradation product thereof, in a biological sample from a subject; and (b) identifying a subject having an elevated level of CD44, POSTN, NES, TERT, UMOD, SGK1, GPR37L1, ISG15, and RGS5, or a byproduct or precursor or degradation product thereof, in the biological sample as compared to a reference level, as having an increased likelihood of developing glioblastoma.

In some instances, the method further comprise (c) determining a level of SPOCD1, DDK1, TNC, GBE1, SMIM3, CLIC1, MT1X, or CYR61, or a byproduct or precursor or degradation product thereof, in a biological sample from a subject; and (d) identifying a subject having an elevated level of SPOCD1, DDK1, TNC, GBE1, SMIM3, CLIC1, MT1X, or CYR61, or a byproduct or precursor or degradation product thereof, in the biological sample as compared to a reference level, as having an increased likelihood of developing glioblastoma.

Also disclosed herein is a method of identifying a subject as having an increased likelihood of developing glioblastoma, wherein the method comprises: (a) determining a level of SPOCD1, DDK1, TNC, GBE1, SMIM3, CLIC1, MT1X, or CYR61, or a byproduct or precursor or degradation product thereof, in a biological sample from a subject; and (b) identifying a subject having an elevated level of SPOCD1, DDK1, TNC, GBE1, SMIM3, CLIC1, MT1X, or CYR61, or a byproduct or precursor or degradation product thereof, in the biological sample as compared to a reference level, as having an increased likelihood of developing glioblastoma.

Also disclosed herein is a method of identifying a subject as having an increased likelihood of developing glioblastoma, wherein the method comprises: (a) determining elevated abundance of IBA1; (b) determining a level of one or more biomarkers selected from DKK1, CHI3L1, HS2ST1, EGR1, TCIM, PLIN2, APOC1, FOS, MGP, SPP1, RPL17, TNC, IFITM3, MT2A, TMSB4X, TMSB10, PDPN, COX6C, VIM, CLIC1, IFITM2, TCEAL9, RPL12, TAGLN, and NAMPT, or RGS5, or a byproduct or precursor or degradation product thereof, in areas of a biological sample from a subject having elevated IBA1 compared to a reference level; and (c) identifying a subject having an elevated level of the one or more biomarkers in the areas as compared to a reference level, as having an increased likelihood of developing glioblastoma.

In some instances, the one or more biomarkers are selected from the group consisting of DKK1, HS2ST1, EGR1, TCIM, FOS, RPL17, TNC, IFITM3, TMSB4X, TMSB10, COX6C, CLIC1, TCEAL9, and RPL12.

Also disclosed herein is a method of identifying a subject as having an increased likelihood of developing glioblastoma, wherein the method comprises: (a) determining a level of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product thereof, in a biological sample from a subject; and (b) identifying a subject having a decreased level of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product thereof, in the biological sample as compared to a reference level, as having an increased likelihood of developing glioblastoma.

In some instances, the methods further include (c) determining a level of NAPB, BASP1, RUNDC3A, NEFM, RAB3A, GNG3, KIF1A, ATP1A3, CNTN1, CELF4, SYN2, TUBB4A, GRIN1, or a byproduct or precursor or degradation product thereof, in a biological sample from a subject; and (d) identifying a subject having a decreased level of NAPB, BASP1, RUNDC3A, NEFM, RAB3A, GNG3, KIF1A, ATP1A3, CNTN1, CELF4, SYN2, TUBB4A, GRIN1, or a byproduct or precursor or degradation product thereof, in the biological sample as compared to a reference level, as having an increased likelihood of developing glioblastoma.

Also disclosed herein is a method of identifying a subject as having an increased likelihood of developing glioblastoma, wherein the method comprises: (a) determining a level of NAPB, BASP1, RUNDC3A, NEFM, RAB3A, GNG3, KIF1A, ATP1A3, CNTN1, CELF4, SYN2, TUBB4A, GRIN1, or a byproduct or precursor or degradation product thereof, in a biological sample from a subject; and (b) identifying a subject having a decreased level of NAPB, BASP1, RUNDC3A, NEFM, RAB3A, GNG3, KIF1A, ATP1A3, CNTN1, CELF4, SYN2, TUBB4A, GRIN1, or a byproduct or precursor or degradation product thereof, in the biological sample as compared to a reference level, as having an increased likelihood of developing glioblastoma.

Also disclosed herein is a method of identifying a subject as having an increased likelihood of developing glioblastoma, wherein the method comprises: (a) determining elevated abundance of IBA1; (b) determining a level of one or more biomarkers selected from DKK1, CHI3L1, HS2ST1, EGR1, TCIM, PLIN2, APOC1, FOS, MGP, SPP1, RPL17, TNC, IFITM3, MT2A, TMSB4X, TMSB10, PDPN, COX6C, VIM, CLIC1, IFITM2, TCEAL9, RPL12, TAGLN, and NAMPT, or RGS5, or a byproduct or precursor or degradation product thereof, in areas of a biological sample from a subject having elevated IBA1 compared to a reference level; and (c) identifying a subject having an elevated level of the one or more biomarkers in the areas as compared to a reference level, as having an increased likelihood of developing glioblastoma.

In some instances, the one or more biomarkers are selected from the group consisting of DKK1, HS2ST1, EGR1, TCIM, FOS, RPL17, TNC, IFITM3, TMSB4X, TMSB10, COX6C, CLIC1, TCEAL9, and RPL12.

In some instances, the method further comprises monitoring the identified subject for the development of symptoms of glioblastoma. In some instances, the method further comprises recording in the identified subject's clinical record that the subject has an increased likelihood or susceptibility of developing glioblastoma. In some instances, the method further comprises notifying the subject's family that the subject has an increased likelihood or susceptibility of developing glioblastoma. In some instances, the method further comprises administering to the subject a treatment for decreasing the rate of progression or decreasing the likelihood or susceptibility of developing glioblastoma. In some instances, the biological sample comprises brain tissue or cerebrospinal fluid. In some instances, the biological sample comprises blood, serum, plasma, or a cell culture sample. In some instances, the methods further include obtaining the biological sample from the subject. In some instances, the level is a level of protein or a byproduct or precursor or degradation product thereof. In some instances, the level is a level of mRNA or a fragment thereof.

Also disclosed herein is a method of monitoring progression of glioblastoma in a subject over time, wherein the method comprises: (a) determining a first level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, or a byproduct or precursor or degradation product thereof, in a first biological sample obtained from a subject at a first time point; (b) determining a second level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, or a byproduct or precursor or degradation product thereof, in a second biological sample obtained from the subject at a second time point; (c) identifying: (i) a subject having an increased second level, as compared to the first level, as having progressing glioblastoma, or (ii) a subject having about the same or a decreased second level as compared to the first level, as having static or regressing glioblastoma.

Also disclosed herein is a method of monitoring progression of glioblastoma in a subject over time, wherein the method comprises: (a) determining a first level of CD44, POSTN, NES, TERT, UMOD, SGK1, GPR37L1, ISG15, or RGS5, or a byproduct or precursor or degradation product thereof, in a first biological sample obtained from a subject at a first time point; (b) determining a second level of CD44, POSTN, NES, TERT, UMOD, SGK1, GPR37L1, ISG15, or RGS5, or a byproduct or precursor or degradation product thereof, in a second biological sample obtained from the subject at a second time point; (c) identifying: (i) a subject having an increased second level, as compared to the first level, as having progressing glioblastoma, or (ii) a subject having about the same or a decreased second level as compared to the first level, as having static or regressing glioblastoma. In some instances, the methods further include (a) determining a first level of SPOCD1, DDK1, TNC, GBE1, SMIM3, CLIC1, MT1X, and CYR61, or a byproduct or precursor or degradation product thereof, in a first biological sample obtained from a subject at a first time point; (b) determining a second level of SPOCD1, DDK1, TNC, GBE1, SMIM3, CLIC1, MT1X, and CYR61, or a byproduct or precursor or degradation product thereof, in a second biological sample obtained from the subject at a second time point; (c) identifying: (i) a subject having an increased second level, as compared to the first level, as having progressing glioblastoma, or (ii) a subject having about the same or a decreased second level as compared to the first level, as having static or regressing glioblastoma.

Also disclosed herein is a method of monitoring progression of glioblastoma in a subject over time, wherein the method comprises: (a) determining a first level of SPOCD1, DDK1, TNC, GBE1, SMIM3, CLIC1, MT1X, and CYR61, or a byproduct or precursor or degradation product thereof, in a first biological sample obtained from a subject at a first time point; (b) determining a second level of SPOCD1, DDK1, TNC, GBE1, SMIM3, CLIC1, MT1X, and CYR61, or a byproduct or precursor or degradation product thereof, in a second biological sample obtained from the subject at a second time point; (c) identifying: (i) a subject having an increased second level, as compared to the first level, as having progressing glioblastoma, or (ii) a subject having about the same or a decreased second level as compared to the first level, as having static or regressing glioblastoma.

Also disclosed herein is a method of monitoring progression of glioblastoma in a subject over time, wherein the method comprises: (a) determining an abundance of IBA1; (b) determining a first level of one or more biomarkers selected from DKK1, CHI3L1, HS2ST1, EGR1, TCIM, PLIN2, APOC1, FOS, MGP, SPP1, RPL17, TNC, IFITM3, MT2A, TMSB4X, TMSB10, PDPN, COX6C, VIM, CLIC1, IFITM2, TCEAL9, RPL12, TAGLN, and NAMPT, or a byproduct or precursor or degradation product thereof, in areas having elevated IBA1 in a first biological sample obtained from a subject at a first time point compared to a reference level; (c) determining a second level of the one or more biomarkers, or a byproduct or precursor or degradation product thereof, in the areas at a second time point; (d) identifying: (i) a subject having an increased second level, as compared to the first level, as having progressing glioblastoma, or (ii) a subject having about the same or a decreased second level as compared to the first level, as having static or regressing glioblastoma.

In some instances, the one or more biomarkers are selected from the group consisting of DKK1, HS2ST1, EGR1, TCIM, FOS, RPL17, TNC, IFITM3, TMSB4X, TMSB10, COX6C, CLIC1, TCEAL9, and RPL12.

Also disclosed herein is a method of monitoring progression of glioblastoma in a subject over time, wherein the method comprises: (a) determining a first level of: GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product thereof, in a first biological sample obtained from a subject at a first time point; (b) determining a second level of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product thereof, in a second biological sample obtained from the subject at a second time point; (c) identifying: (i) a subject having a decreased second level, as compared to the first level, as having progressing glioblastoma, or (ii) a subject having about the same or an increased second level as compared to the first level, as having static or regressing glioblastoma. In some instances, the methods further include (a) determining a first level of NAPB, BASP1, RUNDC3A, NEFM, RAB3A, GNG3, KIF1A, ATP1A3, CNTN1, CELF4, SYN2, TUBB4A, and GRIN1, or a byproduct or precursor or degradation product thereof, in a first biological sample obtained from a subject at a first time point; (b) determining a second level of NAPB, BASP1, RUNDC3A, NEFM, RAB3A, GNG3, KIF1A, ATP1A3, CNTN1, CELF4, SYN2, TUBB4A, and GRIN1, or a byproduct or precursor or degradation product thereof, in a second biological sample obtained from the subject at a second time point; (c) identifying: (i) a subject having a decreased second level, as compared to the first level, as having progressing glioblastoma, or (ii) a subject having about the same or an increased second level as compared to the first level, as having static or regressing glioblastoma.

Also disclosed herein is a method of monitoring progression of glioblastoma in a subject over time, wherein the method comprises: (a) determining a first level of NAPB, BASP1, RUNDC3A, NEFM, RAB3A, GNG3, KIF1A, ATP1A3, CNTN1, CELF4, SYN2, TUBB4A, and GRIN1, or a byproduct or precursor or degradation product thereof, in a first biological sample obtained from a subject at a first time point; (b) determining a second level of SPOCD1, DDK1, TNC, GBE1, SMIM3, CLIC1, MT1X, and CYR61, or a byproduct or precursor or degradation product thereof, in a second biological sample obtained from the subject at a second time point; (c) identifying: (i) a subject having a decreased second level, as compared to the first level, as having progressing glioblastoma, or (ii) a subject having about the same or an increased second level as compared to the first level, as having static or regressing glioblastoma.

Also disclosed herein is a method of monitoring progression of glioblastoma in a subject over time, wherein the method comprises: (a) determining an abundance of IBA1; (b) determining a first level of one or more biomarkers selected from HBA2, HBB, HBA1, COL1A2, MALAT1, RBM25, SLC25A37, NKTR, LUC7L3, ATP1A2, PNISR, MEG3, IFI44L, FAM133B, PNN, PLEKHA4, PTMS, BDP1, MTRNR2L12, SREK1, ARGLU1, XAF1, MTRNR2L8, SRRM2, and COL4A1, or a byproduct or precursor or degradation product thereof, in areas having elevated IBA1 in a first biological sample obtained from a subject at a first time point compared to a reference level; (c) determining a second level of the one or more biomarkers, or a byproduct or precursor or degradation product thereof, in the areas at a second time point; (d) identifying: (i) a subject having an increased second level, as compared to the first level, as having progressing glioblastoma, or (ii) a subject having about the same or a decreased second level as compared to the first level, as having static or regressing glioblastoma. In some instances, the one or more biomarkers are selected from the group consisting of HBA2, HBB, HBA1, MALAT1, RBM25, SLC25A37, NKTR, LUC7L3, PNISR, MEG3, IFI44L, FAM133B, PNN, PLEKHA4, PTMS, BDP1, MTRNR2L12, SREK1, ARGLU1, XAF1, MTRNR2L8, and SRRM2. In some instances, the method comprises identifying a subject as having progressing glioblastoma. In some instances, the method further comprises administering a treatment for glioblastoma to the subject or increasing the dose of a treatment for glioblastoma to be administered to the subject. In some instances, the method further comprises recording in the subject's clinical record that the subject has progressing glioblastoma. In some instances, the method comprises identifying a subject as having static or regressing glioblastoma. In some instances, the method further comprises recording in the subject's clinical record that the subject has static or regressing glioblastoma.

Also disclosed herein is a method of determining efficacy of treatment of a treatment for glioblastoma in a subject, wherein the method comprises: (a) determining a first level of COL1A1, COL3A1, COL8A1, WEE1, CHI3L1, MGP, SRPX, SERPINE1, COL1A2, TIMIP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, or a byproduct or precursor or degradation product thereof, in a first biological sample obtained from a subject at a first time point; (b) determining a second level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, or a byproduct or precursor or degradation product thereof, in a second biological sample obtained from the subject at a second time point, wherein the subject is administered one or more doses of a therapeutic treatment between the first and second time points; (c) identifying: (i) the therapeutic treatment as being effective in a subject having about the same or a decreased second level, as compared to the first level, or (ii) the therapeutic treatment as not being effective in a subject having an increased second level, as compared to the first level.

Also disclosed herein is a method of determining efficacy of treatment of a treatment for glioblastoma in a subject, wherein the method comprises: (a) determining a first level of CD44, POSTN, NES, TERT, UMOD, SGK1, GPR37L1, ISG15, or RGS5, or a byproduct or precursor or degradation product thereof, in a first biological sample obtained from a subject at a first time point; (b) determining a second level of CD44, POSTN, NES, TERT, UMOD, SGK1, GPR37L1, ISG15, or RGS5, or a byproduct or precursor or degradation product thereof, in a second biological sample obtained from the subject at a second time point, wherein the subject is administered one or more doses of a therapeutic treatment between the first and second time points; (c) identifying: (i) the therapeutic treatment as being effective in a subject having about the same or a decreased second level, as compared to the first level, or (ii) the therapeutic treatment as not being effective in a subject having an increased second level, as compared to the first level. In some instances, the methods further include (a) determining a first level of SPOCD1, DDK1, TNC, GBE1, SMIM3, CLIC1, MT1X, and CYR61, or a byproduct or precursor or degradation product thereof, in a first biological sample obtained from a subject at a first time point; (b) determining a second level of SPOCD1, DDK1, TNC, GBE1, SMIM3, CLIC1, MT1X, and CYR61, or a byproduct or precursor or degradation product thereof, in a second biological sample obtained from the subject at a second time point, wherein the subject is administered one or more doses of a therapeutic treatment between the first and second time points; (c) identifying: (i) the therapeutic treatment as being effective in a subject having about the same or a decreased second level, as compared to the first level, or (ii) the therapeutic treatment as not being effective in a subject having an increased second level, as compared to the first level.

Also disclosed herein is a method of determining efficacy of treatment of a treatment for glioblastoma in a subject, wherein the method comprises: (a) determining a first level of SPOCD1, DDK1, TNC, GBE1, SMIM3, CLIC1, MT1X, and CYR61, or a byproduct or precursor or degradation product thereof, in a first biological sample obtained from a subject at a first time point; (b) determining a second level of SPOCD1, DDK1, TNC, GBE1, SMIM3, CLIC1, MT1X, and CYR61, or a byproduct or precursor or degradation product thereof, in a second biological sample obtained from the subject at a second time point, wherein the subject is administered one or more doses of a therapeutic treatment between the first and second time points; (c) identifying: (i) the therapeutic treatment as being effective in a subject having about the same or a decreased second level, as compared to the first level, or (ii) the therapeutic treatment as not being effective in a subject having an increased second level, as compared to the first level.

Also disclosed herein is a method of determining efficacy of treatment of a treatment for glioblastoma in a subject, wherein the method comprises: (a) determining an abundance of IBA1 in a first biological sample; (b) determining a first level of one or more biomarkers selected from DKK1, CHI3L1, HS2ST1, EGR1, TCIM, PLIN2, APOC1, FOS, MGP, SPP1, RPL17, TNC, IFITM3, MT2A, TMSB4X, TMSB10, PDPN, COX6C, VIM, CLIC1, IFITM2, TCEAL9, RPL12, TAGLN, and NAMPT, or a byproduct or precursor or degradation product thereof, in areas having elevated IBA1 in the first biological sample obtained from a subject at a first time point; (c) determining a second level of the one or more biomarkers in a second biological sample in areas having elevated IBA1 obtained from the subject at a second time point, wherein the subject is administered one or more doses of a therapeutic treatment between the first and second time points; (d) identifying: (i) the therapeutic treatment as being effective in a subject having about the same or a decreased second level, as compared to the first level, or (ii) the therapeutic treatment as not being effective in a subject having an increased second level, as compared to the first level. In some instances, the one or more biomarkers are selected from the group consisting of DKK1, HS2ST1, EGR1, TCIM, FOS, RPL17, TNC, IFITM3, TMSB4X, TMSB10, COX6C, CLIC1, TCEAL9, and RPL12.

Also disclosed herein is a method of determining efficacy of treatment of a treatment for glioblastoma in a subject, wherein the method comprises: (a) determining a first level of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product thereof, in a first biological sample obtained from a subject at a first time point; (b) determining a second level of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product thereof, in a second biological sample obtained from the subject at a second time point, wherein the subject is administered one or more doses of a therapeutic treatment between the first and second time points; (c) identifying: (i) the therapeutic treatment as being effective in a subject having an increased second level as compared to the first level, or (ii) the therapeutic treatment as not being effective in a subject having about the same or a decreased second level as compared to the first level. In some instances, the method further includes (a) determining a first level of NAPB, BASP1, RUNDC3A, NEFM, RAB3A, GNG3, KIF1A, ATP1A3, CNTN1, CELF4, SYN2, TUBB4A, GRIN1, or a byproduct or precursor or degradation product thereof, in a first biological sample obtained from a subject at a first time point; (b) determining a second level of NAPB, BASP1, RUNDC3A, NEFM, RAB3A, GNG3, KIF1A, ATP1A3, CNTN1, CELF4, SYN2, TUBB4A, GRIN1, or a byproduct or precursor or degradation product thereof, in a second biological sample obtained from the subject at a second time point, wherein the subject is administered one or more doses of a therapeutic treatment between the first and second time points; (c) identifying: (i) the therapeutic treatment as being effective in a subject having an increased second level as compared to the first level, or (ii) the therapeutic treatment as not being effective in a subject having about the same or a decreased second level as compared to the first level.

Also disclosed herein is a method of determining efficacy of treatment of a treatment for glioblastoma in a subject, wherein the method comprises: (a) determining a first level of NAPB, BASP1, RUNDC3A, NEFM, RAB3A, GNG3, KIF1A, ATP1A3, CNTN1, CELF4, SYN2, TUBB4A, GRIN1, or a byproduct or precursor or degradation product thereof, in a first biological sample obtained from a subject at a first time point; (b) determining a second level of NAPB, BASP1, RUNDC3A, NEFM, RAB3A, GNG3, KIF1A, ATP1A3, CNTN1, CELF4, SYN2, TUBB4A, GRIN1, or a byproduct or precursor or degradation product thereof, in a second biological sample obtained from the subject at a second time point, wherein the subject is administered one or more doses of a therapeutic treatment between the first and second time points; (c) identifying: (i) the therapeutic treatment as being effective in a subject having an increased second level as compared to the first level, or (ii) the therapeutic treatment as not being effective in a subject having about the same or a decreased second level as compared to the first level.

Also disclosed herein is a method of determining efficacy of treatment of a treatment for glioblastoma in a subject, wherein the method comprises: (a) determining an abundance of IBA1 in a first biological sample; (b) determining a first level of one or more biomarkers selected from HBA2, HBB, HBA1, COL1A2, MALAT1, RBM25, SLC25A37, NKTR, LUC7L3, ATP1A2, PNISR, MEG3, IFI44L, FAM133B, PNN, PLEKHA4, PTMS, BDP1, MTRNR2L12, SREK1, ARGLU1, XAF1, MTRNR2L8, SRRM2, and COL4A1, or a byproduct or precursor or degradation product thereof, in a first biological sample obtained from a subject at a first time point; (c) determining a second level of the one or more biomarkers in a second biological sample obtained from the subject at a second time point, wherein the subject is administered one or more doses of a therapeutic treatment between the first and second time points; (d) identifying: (i) the therapeutic treatment as being effective in a subject having an increased second level as compared to the first level, or (ii) the therapeutic treatment as not being effective in a subject having about the same or a decreased second level as compared to the first level. In some instances, the one or more biomarkers are selected from the group consisting of HBA2, HBB, HBA1, MALAT1, RBM25, SLC25A37, NKTR, LUC7L3, PNISR, MEG3, IFI44L, FAM133B, PNN, PLEKHA4, PTMS, BDP1, MTRNR2L12, SREK1, ARGLU1, XAF1, MTRNR2L8, and SRRM2. In some instances, the method comprises identifying the therapeutic treatment as being effective in the subject. In some instances, the method further comprises selecting additional doses of the therapeutic treatment for the subject. In some instances, the method further comprises administering additional doses of the therapeutic treatment to the subject. In some instances, the method further comprises recording in the subject's clinical record that the therapeutic treatment is effective in the subject. In some instances, the method comprises identifying the therapeutic treatment as not being effective in the subject. In some instances, the method further comprises selecting a different therapeutic treatment for the subject. In some instances, the method further comprises administering a different therapeutic treatment to the subject. In some instances, the method further comprises increasing the dose of the therapeutic treatment to be administered to the subject. In some instances, the method further comprises administering one or more additional doses of the therapeutic treatment to the subject in combination with an additional therapeutic treatment. In some instances, the first and second biological samples comprise brain tissue or cerebrospinal fluid. In some instances, the biological sample comprises blood, serum, plasma, or a cell culture sample. In some instances, the method further includes obtaining the first and second biological samples from the subject. In some instances, each of the first and second level is a level of protein or a byproduct or precursor or degradation product thereof. In some instances, each of the first and second level is a level of mRNA or a fragment thereof.

Also disclosed herein is a method of quantitatively profiling gene expression signatures correlating to a disease state of a subject, wherein the disease state is glioblastoma, comprising: generating a profile of expression levels of a plurality of analytes, wherein an analyte in the plurality of analytes is correlated with the glioblastoma in a biological sample obtained from the subject, wherein the profile is generated from a library generated by: (a) contacting the biological sample with an substrate comprising a plurality of attached capture probes, wherein a capture probe of the plurality of attached capture probes comprises (i) the spatial barcode and (ii) a capture domain that binds specifically to a sequence present in the analyte; (b) hybridizing the analyte to the capture domain; (c) extending a 3′ end of the capture probe using the analyte that is specifically bound to the capture domain as a template to generate an extended capture probe; and (d) amplifying the extended capture probe.

Also disclosed herein is a method of treating glioblastoma in a subject in need thereof, comprising administering an effective amount of a therapeutic agent to the subject, wherein the subject has been identified by profiling expression levels of a plurality of analytes, wherein an analyte in the plurality of analytes is correlated with the glioblastoma in a biological sample obtained from the subject, wherein the profile is generated from a library, wherein the library is generated by: (a) contacting the biological sample with an substrate comprising a plurality of attached capture probes, wherein a capture probe of the plurality comprises (i) the spatial barcode and (ii) a capture domain that binds specifically to a sequence present in the analyte; (b) hybridizing the analyte to the capture domain; (c) extending a 3′ end of the capture probe using the analyte that is specifically bound to the capture domain as a template to generate an extended capture probe; and (d) amplifying the extended capture probe.

In some embodiments of the above methods, the analyte of the plurality of analytes are selected from the group consisting of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, and MT-ND5, or a byproduct or precursor or degradation product thereof. In some embodiments, the analyte of the plurality of analytes are selected from the group consisting of CD44, POSTN, NES, TERT, UMOD, SGK1, GPR37L1, ISG15, and RGS5, or a byproduct or precursor or degradation product thereof. In some embodiments, he analyte of the plurality of analytes are selected from the group consisting of SPOCD1, DDK1, TNC, GBE1, SMIM3, CLIC1, MT1X, CYR61, NAPB, BASP1, RUNDC3A, NEFM, RAB3A, GNG3, KIF1A, ATP1A3, CNTN1, CELF4, SYN2, TUBB4A, and GRIN1, or a byproduct or precursor or degradation product thereof. In some embodiments, the methods further comprise determining (i) all or a portion of the sequence of the spatial barcode or the complement thereof, and (ii) all or a portion of the sequence of the analyte from the biological sample or the capture agent barcode domain. In some embodiments, the method further comprises using the determined sequences of (i) and (ii) to identify the location of the analyte in the biological sample.

Also disclosed herein is a kit comprising: an antibody that binds specifically to COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, or a byproduct or precursor or degradation product thereof, or any combination thereof, and instructions for performing the methods disclosed herein.

Also disclosed herein is a kit comprising: an antibody that binds specifically to COL1A1, COL3A1, COL8A1, WEE1, CHI3L1, MGP, SRPX, SERPINE1, COL1A2, TIMIP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, CD44, POSTN, NES, TERT, UMOD, SGK1, GPR37L1, ISG15, RGS5, or a byproduct or precursor or degradation product thereof, or any combination thereof, and instructions for performing the methods disclosed herein.

Also disclosed herein is a kit comprising: an antibody that binds specifically to COL1A1, COL3A1, COL8A1, WEE1, CHI3L1, MGP, SRPX, SERPINE1, COL1A2, TIMIP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, CD44, POSTN, NES, TERT, UMOD, SGK1, GPR37L1, ISG15, RGS5, SPOCD1, DDK1, TNC, GBE1, SMIM3, CLIC1, MT1X, CYR61, NAPB, BASP1, RUNDC3A, NEFM, RAB3A, GNG3, KIF1A, ATP1A3, CNTN1, CELF4, SYN2, TUBB4A, GRIN1, DKK1, HS2ST1, EGR1, TCIM, FOS, RPL17, TNC, IFITM3, TMSB4X, TMSB10, COX6C, CLIC1, TCEAL9, RPL12, HBA2, HBB, HBA1, MALAT1, RBM25, SLC25A37, NKTR, LUC7L3, PNISR, MEG3, IFI44L, FAM133B, PNN, PLEKHA4, PTMS, BDP1, MTRNR2L12, SREK1, ARGLU1, XAF1, MTRNR2L8, SRRM2, or a byproduct or precursor or degradation product thereof, or any combination thereof, and instructions for performing the methods disclosed herein.

Also disclosed herein is a method of identifying a patient subpopulation for which a therapeutic treatment is effective for glioblastoma, the method comprising: (a) administering a therapeutic treatment for glioblastoma to a patient subpopulation; (b) determining (i) a first level of one or more biomarkers selected from COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, or a byproduct or precursor or degradation product thereof, in first biological samples obtained from a patient subpopulation at a first time point and (ii) a second level of the one or more biomarkers in second biological samples obtained from the patient population at a second time point, wherein the patient subpopulation is administered one or more doses of a therapeutic treatment between the first and second time points; (c) determining a correlation between efficacy of the therapeutic treatment and the second level in samples from the patient subpopulation as compared to the level in a sample obtained from an untreated patient, wherein a lower second level in the samples from the patient subpopulation as compared to the level in the sample from the untreated patient is indicative that the therapeutic treatment is effective for glioblastoma in the patient subpopulation.

Also disclosed herein is a method of identifying a patient subpopulation for which a therapeutic treatment is effective for glioblastoma, the method comprising: (a) administering a therapeutic treatment for glioblastoma to a patient subpopulation; (b) determining (i) a first level of one or more biomarkers selected from COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, CD44, POSTN, NES, TERT, UMOD, SGK1, GPR37L1, ISG15, or RGS5, or a byproduct or precursor or degradation product thereof, in first biological samples obtained from a patient subpopulation at a first time point and (ii) a second level of the one or more biomarkers, or a byproduct or precursor or degradation product thereof, in second biological samples obtained from the patient population at a second time point, wherein the patient subpopulation is administered one or more doses of a therapeutic treatment between the first and second time points; (c) determining a correlation between efficacy of the therapeutic treatment and the second level in samples from the patient subpopulation as compared to the level in a sample obtained from an untreated patient, wherein a lower second level in the samples from the patient subpopulation as compared to the level in the sample from the untreated patient is indicative that the therapeutic treatment is effective for glioblastoma in the patient subpopulation.

In some instances, the methods further include (a) administering a therapeutic treatment for glioblastoma to a patient subpopulation; (b) determining (i) a first level of one or more of SPOCD1, DDK1, TNC, GBE1, SMIM3, CLIC1, MT1X, or CYR61, or a byproduct or precursor or degradation product thereof, in first biological samples obtained from a patient subpopulation at a first time point and (ii) a second level of one or more of SPOCD1, DDK1, TNC, GBE1, SMIM3, CLIC1, MT1X, or CYR61, or a byproduct or precursor or degradation product thereof, in second biological samples obtained from the patient population at a second time point, wherein the patient subpopulation is administered one or more doses of a therapeutic treatment between the first and second time points; (c) determining a correlation between efficacy of the therapeutic treatment and the second level in samples from the patient subpopulation as compared to the level in a sample obtained from an untreated patient, wherein a lower second level in the samples from the patient subpopulation as compared to the level in the sample from the untreated patient is indicative that the therapeutic treatment is effective for glioblastoma in the patient subpopulation.

Also disclosed herein is a method of identifying a patient subpopulation for which a therapeutic treatment is effective for glioblastoma, the method comprising: (a) administering a therapeutic treatment for glioblastoma to a patient subpopulation; (b) determining (i) a first level of one or more biomarkers selected from SPOCD1, DDK1, TNC, GBE1, SMIM3, CLIC1, MT1X, or CYR61, or a byproduct or precursor or degradation product thereof, in first biological samples obtained from a patient subpopulation at a first time point and (ii) a second level of the one or more biomarkers, or a byproduct or precursor or degradation product thereof, in second biological samples obtained from the patient population at a second time point, wherein the patient subpopulation is administered one or more doses of a therapeutic treatment between the first and second time points; (c) determining a correlation between efficacy of the therapeutic treatment and the second level in samples from the patient subpopulation as compared to the level in a sample obtained from an untreated patient, wherein a lower second level in the samples from the patient subpopulation as compared to the level in the sample from the untreated patient is indicative that the therapeutic treatment is effective for glioblastoma in the patient subpopulation.

Also disclosed herein is a method of identifying a patient subpopulation for which a therapeutic treatment is effective for glioblastoma, the method comprising: (a) administering a therapeutic treatment for glioblastoma to a patient subpopulation; (b) determining abundance of IBA1; (c) determining (i) a first level of one or more biomarkers selected from DKK1, CHI3L1, HS2ST1, EGR1, TCIM, PLIN2, APOC1, FOS, MGP, SPP1, RPL17, TNC, IFITM3, MT2A, TMSB4X, TMSB10, PDPN, COX6C, VIM, CLIC1, IFITM2, TCEAL9, RPL12, TAGLN, and NAMPT, or a byproduct or precursor or degradation product thereof, in areas of the first biological samples with elevated IBA1 obtained from a patient subpopulation at a first time point and (ii) a second level of the one or more biomarkers, or a byproduct or precursor or degradation product thereof, in second biological samples obtained from the patient population at a second time point in the areas, wherein the patient subpopulation is administered one or more doses of a therapeutic treatment between the first and second time points; (d) determining a correlation between efficacy of the therapeutic treatment and the second level in samples from the patient subpopulation as compared to the level in a sample obtained from an untreated patient, wherein a lower second level in the samples from the patient subpopulation as compared to the level in the sample from the untreated patient is indicative that the therapeutic treatment is effective for glioblastoma in the patient subpopulation. In some instances, the one or more biomarkers are selected from the group consisting of DKK1, HS2ST1, EGR1, TCIM, FOS, RPL17, TNC, IFITM3, TMSB4X, TMSB10, COX6C, CLIC1, TCEAL9, and RPL12. In some instances, the therapeutic treatment is an antagonist of the gene, or a byproduct or precursor or degradation product thereof.

Also disclosed herein is a method of identifying a patient subpopulation for which a therapeutic treatment is effective for glioblastoma, the method comprising: (a) administering a therapeutic treatment for glioblastoma to a patient subpopulation; (b) determining (i) a first level of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product thereof, in first biological samples obtained from a patient subpopulation at a first time point and (ii) a second level of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product thereof, in second biological samples obtained from the patient population at a second time point, wherein the patient subpopulation is administered one or more doses of a therapeutic treatment between the first and second time points; (c) determining a correlation between efficacy of the therapeutic treatment and the second level in samples from the patient subpopulation as compared to a level in a sample obtained from an untreated patient, wherein about the same or an elevated second level in the samples from the patient subpopulation as compared to the level in the sample from the untreated patient is indicative that the therapeutic treatment is effective for glioblastoma in the patient subpopulation. In some instances, the therapeutic treatment is an agonist of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product thereof. In some instances, the method further includes (a) administering a therapeutic treatment for glioblastoma to a patient subpopulation; (b) determining (i) a first level of one or more of NAPB, BASP1, RUNDC3A, NEFM, RAB3A, GNG3, KIF1A, ATP1A3, CNTN1, CELF4, SYN2, TUBB4A, GRIN1, or a byproduct or precursor or degradation product thereof, in first biological samples obtained from a patient subpopulation at a first time point and (ii) a second level of one or more of NAPB, BASP1, RUNDC3A, NEFM, RAB3A, GNG3, KIF1A, ATP1A3, CNTN1, CELF4, SYN2, TUBB4A, GRIN1, or a byproduct or precursor or degradation product thereof, in second biological samples obtained from the patient population at a second time point, wherein the patient subpopulation is administered one or more doses of a therapeutic treatment between the first and second time points; (c) determining a correlation between efficacy of the therapeutic treatment and the second level in samples from the patient subpopulation as compared to a level in a sample obtained from an untreated patient, wherein about the same or an elevated second level in the samples from the patient subpopulation as compared to the level in the sample from the untreated patient is indicative that the therapeutic treatment is effective for glioblastoma in the patient subpopulation.

Also disclosed herein is a method of identifying a patient subpopulation for which a therapeutic treatment is effective for glioblastoma, the method comprising: (a) administering a therapeutic treatment for glioblastoma to a patient subpopulation; (b) determining (i) a first level of one or more biomarkers selected from NAPB, BASP1, RUNDC3A, NEFM, RAB3A, GNG3, KIF1A, ATP1A3, CNTN1, CELF4, SYN2, TUBB4A, GRIN1, or a byproduct or precursor or degradation product thereof, in first biological samples obtained from a patient subpopulation at a first time point and (ii) a second level of the one or more biomarkers, or a byproduct or precursor or degradation product thereof, in second biological samples obtained from the patient population at a second time point, wherein the patient subpopulation is administered one or more doses of a therapeutic treatment between the first and second time points; (c) determining a correlation between efficacy of the therapeutic treatment and the second level in samples from the patient subpopulation as compared to a level in a sample obtained from an untreated patient, wherein about the same or an elevated second level in the samples from the patient subpopulation as compared to the level in the sample from the untreated patient is indicative that the therapeutic treatment is effective for glioblastoma in the patient subpopulation.

Also disclosed herein is a method of identifying a patient subpopulation for which a therapeutic treatment is effective for glioblastoma, the method comprising: (a) administering a therapeutic treatment for glioblastoma to a patient subpopulation; (b) determining abundance of IBA1; (c) determining (i) a first level of one or more biomarkers selected from HBA2, HBB, HBA1, COL1A2, MALAT1, RBM25, SLC25A37, NKTR, LUC7L3, ATP1A2, PNISR, MEG3, IFI44L, FAM133B, PNN, PLEKHA4, PTMS, BDP1, MTRNR2L12, SREK1, ARGLU1, XAF1, MTRNR2L8, SRRM2, and COL4A1, or a byproduct or precursor or degradation product thereof, in areas of the first biological samples with elevated IBA1 obtained from a patient subpopulation at a first time point and (ii) a second level of the one or more biomarkers, or a byproduct or precursor or degradation product thereof, in second biological samples obtained from the patient population at a second time point in the areas, wherein the patient subpopulation is administered one or more doses of a therapeutic treatment between the first and second time points; (d) determining a correlation between efficacy of the therapeutic treatment and the second level in samples from the patient subpopulation as compared to a level in a sample obtained from an untreated patient, wherein about the same or an elevated second level in the samples from the patient subpopulation as compared to the level in the sample from the untreated patient is indicative that the therapeutic treatment is effective for glioblastoma in the patient subpopulation. In some instances, the one or more biomarkers are selected from the group consisting of HBA2, HBB, HBA1, MALAT1, RBM25, SLC25A37, NKTR, LUC7L3, PNISR, MEG3, IFI44L, FAM133B, PNN, PLEKHA4, PTMS, BDP1, MTRNR2L12, SREK1, ARGLU1, XAF1, MTRNR2L8, and SRRM2.

Also disclosed herein is a method of modifying treatment of a glioblastoma patient with a therapeutic treatment, the method comprising: (a) administering a therapeutic treatment to a glioblastoma patient; (b) determining (i) a pre-treatment level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, CD44, POSTN, NES, TERT, UMOD, SGK1, GPR37L1, ISG15, or RGS5, or a byproduct or precursor or degradation product thereof, in a pre-treatment sample obtained from the glioblastoma patient before treatment and (ii) a post-treatment level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, or a byproduct or precursor or degradation product thereof, in a post-treatment sample obtained from the glioblastoma patient after treatment, wherein a higher post-treatment level, as compared to the pre-treatment level, is indicative of the responsiveness to treatment with the therapeutic treatment; and (c) increasing the amount of the therapeutic treatment administered to the patient based on the higher post-treatment level as compared to the pre-treatment level. In some instances, the therapeutic treatment is an antagonist of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, CD44, POSTN, NES, TERT, UMOD, SGK1, GPR37L1, ISG15, or RGS5, or a byproduct or precursor or degradation product thereof.

Also disclosed herein is a method of modifying treatment of a glioblastoma patient with a therapeutic treatment, the method comprising: (a) administering a therapeutic treatment to a glioblastoma patient; (b) determining (i) a pre-treatment level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, CD44, POSTN, NES, TERT, UMOD, SGK1, GPR37L1, ISG15, or RGS5, or a byproduct or precursor or degradation product thereof, in a pre-treatment sample obtained from the glioblastoma patient before treatment and (ii) a post-treatment level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, CD44, POSTN, NES, TERT, UMOD, SGK1, GPR37L1, ISG15, or RGS5, or a byproduct or precursor or degradation product thereof, in a post-treatment sample obtained from the glioblastoma patient after treatment, wherein a higher post-treatment level, as compared to the pre-treatment level, is indicative of the responsiveness to treatment with the therapeutic treatment; and (c) increasing the amount of the therapeutic treatment administered to the patient based on the higher post-treatment level as compared to the pre-treatment level. In some instances, the therapeutic treatment is an antagonist of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, CD44, POSTN, NES, TERT, UMOD, SGK1, GPR37L1, ISG15, or RGS5, CD44, POSTN, NES, TERT, UMOD, SGK1, GPR37L1, ISG15, RGS5, or a byproduct or precursor or degradation product thereof.

Also disclosed herein is a method of modifying treatment of a glioblastoma patient with a therapeutic treatment, the method comprising: (a) administering a therapeutic treatment to a glioblastoma patient; (b) determining (i) a pre-treatment level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, CD44, POSTN, NES, TERT, UMOD, SGK1, GPR37L1, ISG15, or RGS5, or a byproduct or precursor or degradation product thereof, in a pre-treatment sample obtained from the glioblastoma patient before treatment and (ii) a post-treatment level of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, CD44, POSTN, NES, TERT, UMOD, SGK1, GPR37L1, ISG15, RGS5, SPOCD1, DDK1, TNC, GBE1, SMIM3, CLIC1, MT1X, or CYR61, or a byproduct or precursor or degradation product thereof, in a post-treatment sample obtained from the glioblastoma patient after treatment, wherein a higher post-treatment level, as compared to the pre-treatment level, is indicative of the responsiveness to treatment with the therapeutic treatment; and (c) increasing the amount of the therapeutic treatment administered to the patient based on the higher post-treatment level as compared to the pre-treatment level. In some instances, the therapeutic treatment is an antagonist of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, CD44, POSTN, NES, TERT, UMOD, SGK1, GPR37L1, ISG15, RGS5, CD44, POSTN, NES, TERT, UMOD, SGK1, GPR37L1, ISG15, RGS5, SPOCD1, DDK1, TNC, GBE1, SMIM3, CLIC1, MT1X, or CYR61, or a byproduct or precursor or degradation product thereof.

Also disclosed herein is a method of modifying treatment of a glioblastoma patient with a therapeutic treatment, the method comprising: (a) administering a therapeutic treatment to a glioblastoma patient; (b) determining the abundance of IBA1; (c) determining (i) a pre-treatment level of one or more biomarkers selected from DKK1, CHI3L1, HS2ST1, EGR1, TCIM, PLIN2, APOC1, FOS, MGP, SPP1, RPL17, TNC, IFITM3, MT2A, TMSB4X, TMSB10, PDPN, COX6C, VIM, CLIC1, IFITM2, TCEAL9, RPL12, TAGLN, and NAMPT, or a byproduct or precursor or degradation product thereof, in areas of a pre-treatment sample having elevated IBA1 obtained from the glioblastoma patient before treatment and (ii) a post-treatment level of the one or more biomarkers in the areas in a post-treatment sample obtained from the glioblastoma patient after treatment, wherein a higher post-treatment level, as compared to the pre-treatment level, is indicative of the responsiveness to treatment with the therapeutic treatment; and (c) increasing the amount of the therapeutic treatment administered to the patient based on the higher post-treatment level as compared to the pre-treatment level. In some instances, the one or more biomarkers is selected from the group consisting of DKK1, HS2ST1, EGR1, TCIM, FOS, RPL17, TNC, IFITM3, TMSB4X, TMSB10, COX6C, CLIC1, TCEAL9, and RPL12.

Also disclosed herein is a method of modifying treatment of a glioblastoma patient with a therapeutic treatment, the method comprising: (a) administering a therapeutic treatment to a glioblastoma patient; (b) determining (i) a pre-treatment level of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product thereof, in a pre-treatment sample obtained from the glioblastoma patient before treatment and (ii) a post-treatment level of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, or MT-ND5, or a byproduct or precursor or degradation product thereof, in a post-treatment sample obtained from the glioblastoma patient after treatment, wherein a decreased post-treatment level as compared to the pre-treatment level, is indicative of the responsiveness to treatment with the therapeutic treatment; and (c) increasing the amount of the therapeutic treatment administered to the patient based on the decreased post-treatment level as compared to the pre-treatment level.

Also disclosed herein is a method of modifying treatment of a glioblastoma patient with a therapeutic treatment, the method comprising: (a) administering a therapeutic treatment to a glioblastoma patient; (b) determining (i) a pre-treatment level of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, NAPB, BASP1, RUNDC3A, NEFM, RAB3A, GNG3, KIF1A, ATP1A3, CNTN1, CELF4, SYN2, TUBB4A, GRIN1, or a byproduct or precursor or degradation product thereof, in a pre-treatment sample obtained from the glioblastoma patient before treatment and (ii) a post-treatment level of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, NAPB, BASP1, RUNDC3A, NEFM, RAB3A, GNG3, KIF1A, ATP1A3, CNTN1, CELF4, SYN2, TUBB4A, GRIN1, or a byproduct or precursor or degradation product thereof, in a post-treatment sample obtained from the glioblastoma patient after treatment, wherein a decreased post-treatment level as compared to the pre-treatment level, is indicative of the responsiveness to treatment with the therapeutic treatment; and (c) increasing the amount of the therapeutic treatment administered to the patient based on the decreased post-treatment level as compared to the pre-treatment level.

Also disclosed herein is a method of modifying treatment of a glioblastoma patient with a therapeutic treatment, the method comprising: (a) administering a therapeutic treatment to a glioblastoma patient; (b) determining the abundance of IBA1; (c) determining (i) a pre-treatment level of one or more biomarkers selected from HBA2, HBB, HBA1, COL1A2, MALAT1, RBM25, SLC25A37, NKTR, LUC7L3, ATP1A2, PNISR, MEG3, IFI44L, FAM133B, PNN, PLEKHA4, PTMS, BDP1, MTRNR2L12, SREK1, ARGLU1, XAF1, MTRNR2L8, SRRM2, and COL4A1, or a byproduct or precursor or degradation product thereof, in areas of a pre-treatment sample having elevated IBA1 obtained from the glioblastoma patient before treatment and (ii) a post-treatment level of the one or more biomarkers in the areas in a post-treatment sample obtained from the glioblastoma patient after treatment, wherein a decreased post-treatment level as compared to the pre-treatment level, is indicative of the responsiveness to treatment with the therapeutic treatment; and (d) increasing the amount of the therapeutic treatment administered to the patient based on the decreased post-treatment level as compared to the pre-treatment level. In some instances, the one or more biomarkers is selected from the group consisting of HBA2, HBB, HBA1, MALAT1, RBM25, SLC25A37, NKTR, LUC7L3, PNISR, MEG3, IFI44L, FAM133B, PNN, PLEKHA4, PTMS, BDP1, MTRNR2L12, SREK1, ARGLU1, XAF1, MTRNR2L8, and SRRM2.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise. Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

### DESCRIPTION OF DRAWINGS

The patent or application file contains at least one drawing executed in color. Copies of this patent or patent application publication with color drawing(s) will be provided by the Office upon request and payment of the necessary fee.

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is a schematic illustrating a cleavable capture probe, wherein the cleaved capture probe can enter into a non-permeabilized cell and bind to target analytes within the sample.

FIG. 3 is a schematic diagram of an exemplary multiplexed spatially-barcoded feature.

FIG. 4 is a schematic diagram of an exemplary analyte capture agent.

FIG. 5 is a schematic diagram depicting an exemplary interaction between a feature-immobilized capture probe 524 and an analyte capture agent 526.

FIGS. 6A, 6B, and 6C are schematics illustrating how streptavidin cell tags can be utilized in an array-based system to produce a spatially-barcoded cells or cellular contents.

FIG. 7A shows a histological section of a human cerebral cortex (unspecified) sample.

FIG. 7B shows a tissue plot with spots colored by unsupervised clustering.

FIG. 8A shows a histological section of a human cerebral cortex (temporal) sample.

FIG. 8B shows a tissue plot with spots colored by unsupervised clustering.

FIG. 9A shows a histological section of a human spinal cord sample.

FIG. 9B shows a tissue plot with spots colored by unsupervised clustering.

FIG. 10A shows a histological section of a human cerebellum sample.

FIG. 10B shows a tissue plot with spots colored by unsupervised clustering.

FIG. 11A is a t-SNE plot of spots colored by unsupervised clustering.

FIG. 11B is a UMAP plot of spots colored by unsupervised clustering.

FIG. 12 is a scatter plot showing the differential expression of genes.

FIG. 13A is a t-SNE plot of spots colored by unsupervised clustering.

FIG. 13B is a UMAP plot of spots colored by unsupervised clustering.

FIG. 14 is a scatter plot showing the differential expression of genes.

FIG. 15A is a t-SNE plot of spots colored by unsupervised clustering.

FIG. 15B is a UMAP plot of spots colored by unsupervised clustering.

FIG. 16A is a table illustrating differential gene expression in glioblastoma and healthy brain samples.

FIG. 16B is a scatter plot illustrating differential gene expression in glioblastoma and healthy brain samples.

FIG. 17A is a table illustrating differential gene expression in glioblastoma and healthy brain samples.

FIG. 17B is a scatter plot illustrating differential gene expression in glioblastoma and healthy brain samples.

FIG. 18A is a table illustrating differential gene expression in glioblastoma and healthy brain samples.

FIG. 18B is a table illustrating differential gene expression in glioblastoma and healthy brain samples.

FIG. 19 shows tissue plots from glioblastoma samples with spots colored by unsupervised clustering.

FIG. 20A shows a representative H&E stain image for a glioblastoma sample.

FIG. 20B shows cluster expression data for eight different clusters in a glioblastoma sample.

FIG. 20C shows a t-SNE plot of gene expression measurements within each spot on the gene expression array in a glioblastoma sample.

FIG. 21A shows a representative H&E stain image from a normal sample.

FIG. 21B shows cluster expression data for seven different clusters in a normal sample.

FIG. 21C shows a t-SNE plot of gene expression measurements within each spot on the gene expression array in a normal sample.

FIG. 22A shows a representative H&E stain image from a normal sample.

FIG. 22B shows cluster expression data for eight different clusters in a normal sample.

FIG. 22C shows a t-SNE plot of gene expression measurements within each spot on the gene expression array in a normal sample.

FIGS. 23A-23H show upregulation of CD44, POSTN, NES, TERT, UMOD, SGK1, GPR37L1, and ISG15, respectively in a glioblastoma sample.

FIG. 24 shows co-localized upregulation of ISG15, UMOD, SGK1, RGS5, and GPR37L1 in a glioblastoma sample. Numbers in parentheses represent the number of spots (out of 5000 total spots) where gene expression was detected.

FIGS. 25A-25B show immunofluorescence detection of GFAP (FIG. 25A) and IBA1 (FIG. 25B) in a glioblastoma sample.

FIG. 26A shows co-localized expression of GFAP protein and GFAP mRNA.

FIG. 26B shows co-localized expression of IBA1 protein and IBA1 mRNA.

FIG. 26C shows cluster expression data for nine different clusters in a glioblastoma sample.

FIG. 26D shows spot expression of IBA1, showing spots with high expression of IBA1 and spots with low expression of IBA1.

FIG. 27A is a table illustrating differential gene expression in glioblastoma and healthy brain samples.

FIG. 27B is a scatter plot illustrating differential gene expression in glioblastoma and healthy brain samples.

FIG. 28A is a table illustrating differential gene expression in glioblastoma and healthy brain samples.

FIG. 28B is a scatter plot illustrating differential gene expression in glioblastoma and healthy brain samples.

FIG. 29A is a table illustrating differential gene expression in glioblastoma and healthy brain samples.

FIG. 29B is a table illustrating differential gene expression in glioblastoma and healthy brain samples.

FIG. 30 shows tissue plots from glioblastoma samples with spots colored by unsupervised clustering.

FIG. 31 shows a heat map of differentially expressed biomarkers in glioblastoma samples. Scale is shown as a log 2 fold change.

FIG. 32 shows a heat map of differentially expressed biomarkers that correlate with differential expression of IBA1 in glioblastoma samples. Scale is shown as a log 2 fold change.

### DETAILED DESCRIPTION

### I. Introduction

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a connected probe (e.g., a ligation product) or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)).

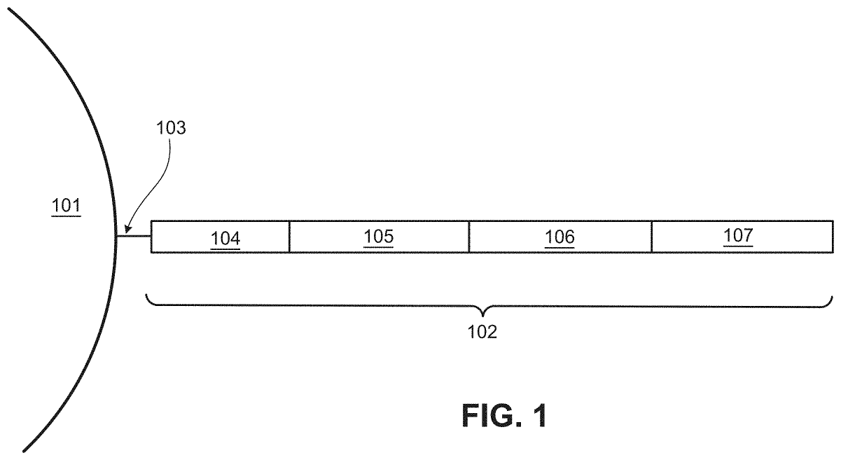

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that are useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. In some embodiments, the capture probe comprises an additional functional sequence that can be located, e.g., between spatial barcode 105 and UMI sequence 106, between UMI sequence 106 and capture domain 107, or following capture domain 107. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 is common to all of the probes attached to a given feature. In some embodiments, the UMI sequence 106 of a capture probe attached to a given feature is different from the UMI sequence of a different capture probe attached to the given feature.

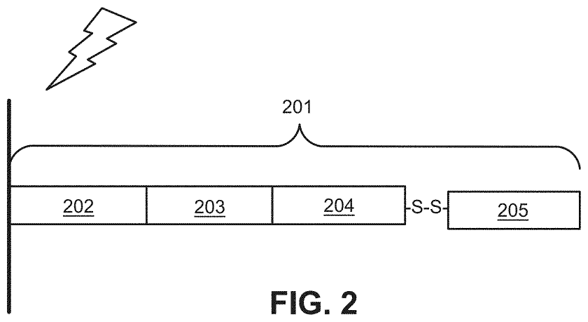

FIG. 2 is a schematic illustrating a cleavable capture probe, wherein the cleaved capture probe can enter into a non-permeabilized cell and bind to analytes within the sample. The capture probe 201 contains a cleavage domain 202, a cell penetrating peptide 203, a reporter molecule 204, and a disulfide bond (—S—S—). 205 represents all other parts of a capture probe, for example a spatial barcode and a capture domain.

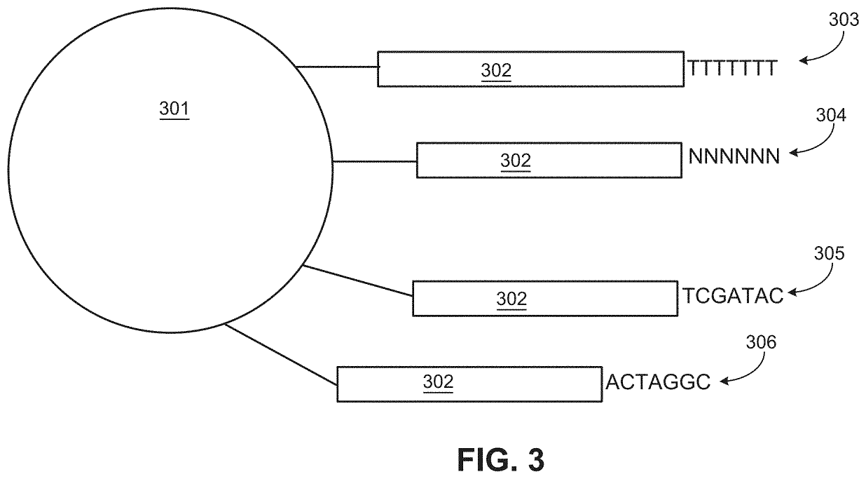

FIG. 3 is a schematic diagram of an exemplary multiplexed spatially-barcoded feature. In FIG. 3, the feature 301 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 302. One type of capture probe associated with the feature includes the spatial barcode 302 in combination with a poly(T) capture domain 303, designed to capture mRNA target analytes. A second type of capture probe associated with the feature includes the spatial barcode 302 in combination with a random N-mer capture domain 304 for gDNA analysis. A third type of capture probe associated with the feature includes the spatial barcode 302 in combination with a capture domain complementary to a capture handle sequence of an analyte capture agent of interest 305. A fourth type of capture probe associated with the feature includes the spatial barcode 302 in combination with a capture domain that can specifically bind a nucleic acid molecule 306 that can function in a CRISPR assay (e.g., CRISPR/Cas9). While only four different capture probe-barcoded constructs are shown in FIG. 3, capture-probe barcoded constructs can be tailored for analyses of any given analyte associated with a nucleic acid and capable of binding with such a construct. For example, the schemes shown in FIG. 3 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) a capture handle sequence. As used herein, the term “analyte binding moiety barcode” refers to a barcode that is associated with or otherwise identifies the analyte binding moiety. As used herein, the term “analyte capture sequence” or “capture handle sequence” refers to a region or moiety configured to hybridize to, bind to, couple to, or otherwise interact with a capture domain of a capture probe. In some embodiments, a capture handle sequence is complementary to a capture domain of a capture probe. In some cases, an analyte binding moiety barcode (or portion thereof) may be able to be removed (e.g., cleaved) from the analyte capture agent.

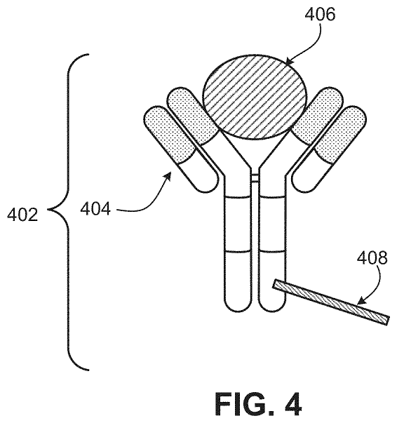

FIG. 4 is a schematic diagram of an exemplary analyte capture agent 402 comprised of an analyte-binding moiety 404 and an analyte-binding moiety barcode domain 408. The exemplary analyte-binding moiety 404 is a molecule capable of binding to an analyte 406 and the analyte capture agent is capable of interacting with a spatially-barcoded capture probe. The analyte-binding moiety can bind to the analyte 406 with high affinity and/or with high specificity. The analyte capture agent can include an analyte-binding moiety barcode domain 408, a nucleotide sequence (e.g., an oligonucleotide), which can hybridize to at least a portion or an entirety of a capture domain of a capture probe. The analyte-binding moiety barcode domain 408 can comprise an analyte binding moiety barcode and a capture handle sequence described herein. The analyte-binding moiety 404 can include a polypeptide and/or an aptamer. The analyte-binding moiety 404 can include an antibody or antibody fragment (e.g., an antigen-binding fragment).

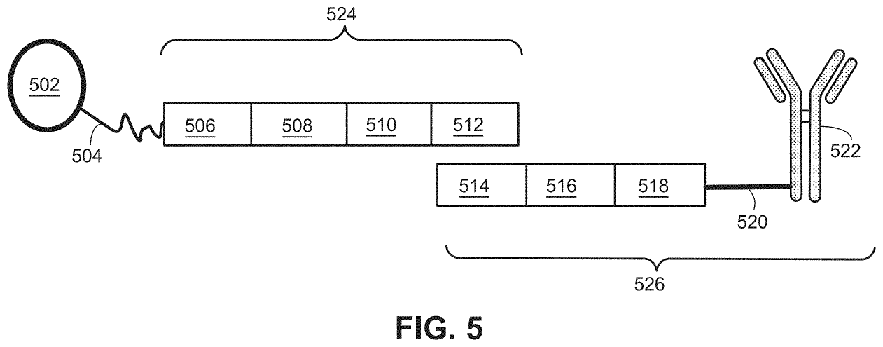

FIG. 5 is a schematic diagram depicting an exemplary interaction between a feature-immobilized capture probe 524 and an analyte capture agent 526. The feature-immobilized capture probe 524 can include a spatial barcode 508 as well as functional sequences 506 and UMI 510, as described elsewhere herein. The capture probe can also include a capture domain 512 that is capable of binding to an analyte capture agent 526. The analyte capture agent 526 can include a functional sequence 518, analyte binding moiety barcode 516, and a capture handle sequence 514 that is capable of binding to the capture domain 512 of the capture probe 524. The analyte capture agent can also include a linker 520 that allows the capture agent barcode domain 516 to couple to the analyte binding moiety 522.

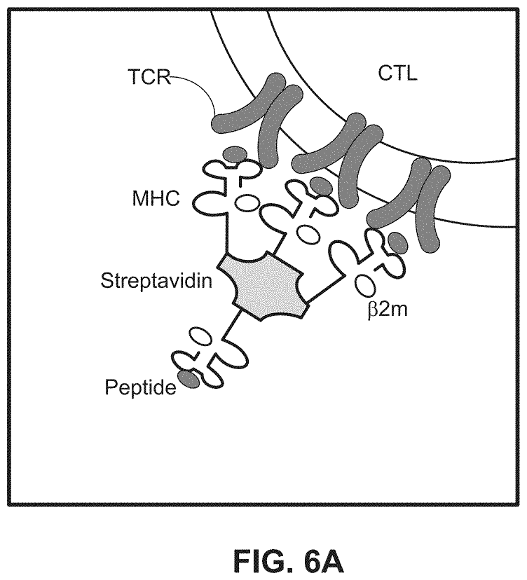

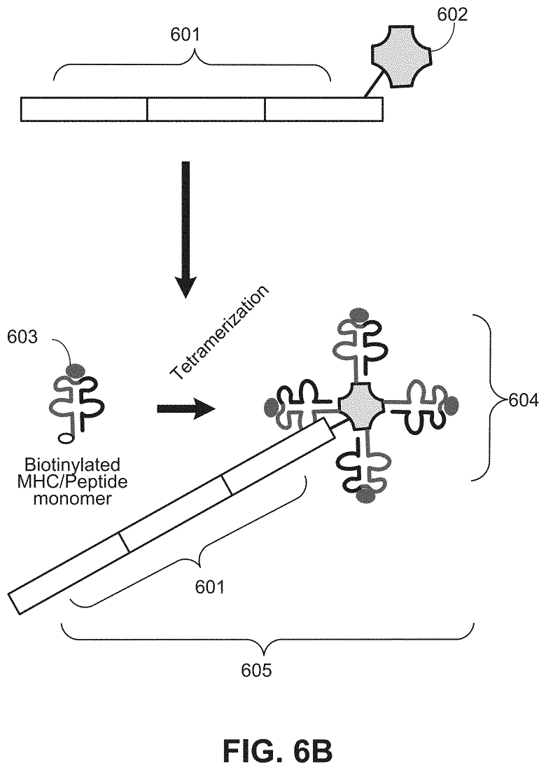

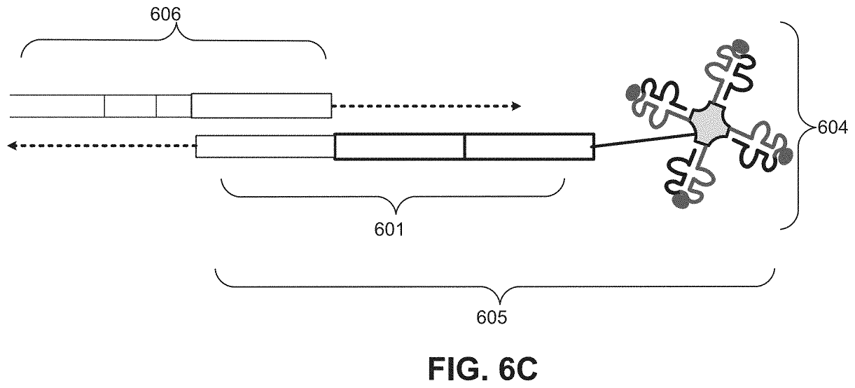

FIGS. 6A, 6B, and 6C are schematics illustrating how streptavidin cell tags can be utilized in an array-based system to produce a spatially-barcoded cell or cellular contents. For example, as shown in FIG. 6A, peptide-bound major histocompatibility complex (MHC) can be individually associated with biotin (β2m) and bound to a streptavidin moiety such that the streptavidin moiety comprises multiple pMHC moieties. Each of these moieties can bind to a TCR such that the streptavidin binds to a target T-cell via multiple MCH/TCR binding interactions. Multiple interactions synergize and can substantially improve binding affinity. Such improved affinity can improve labelling of T-cells and also reduce the likelihood that labels will dissociate from T-cell surfaces. As shown in FIG. 6B, a capture agent barcode domain 601 can be modified with streptavidin 602 and contacted with multiple molecules of biotinylated MHC 603 such that the biotinylated MHC 603 molecules are coupled with the streptavidin conjugated capture agent barcode domain 601. The result is a barcoded MHC multimer complex 1105. As shown in FIG. 6B, the capture agent barcode domain sequence 601 can identify the MHC as its associated label and also includes optional functional sequences such as sequences for hybridization with other oligonucleotides. As shown in FIG. 6C, one example oligonucleotide is capture probe 606 that comprises a complementary sequence (e.g., rGrGrG corresponding to C C C), a barcode sequence and other functional sequences, such as, for example, a UMI, an adapter sequence (e.g., comprising a sequencing primer sequence (e.g., R1 or a partial R1 (“pR1”), R2), a flow cell attachment sequence (e.g., P5 or P7 or partial sequences thereof)), etc. In some cases, capture probe 606 may at first be associated with a feature (e.g., a gel bead) and released from the feature. In other embodiments, capture probe 606 can hybridize with a capture agent barcode domain 601 of the MHC-oligonucleotide complex 605. The hybridized oligonucleotides (Spacer C C C and Spacer rGrGrG) can then be extended in primer extension reactions such that constructs comprising sequences that correspond to each of the two spatial barcode sequences (the spatial barcode associated with the capture probe, and the barcode associated with the MHC-oligonucleotide complex) are generated. In some cases, one or both of these corresponding sequences may be a complement of the original sequence in capture probe 606 or capture agent barcode domain 601. In other embodiments, the capture probe and the capture agent barcode domain are ligated together. The resulting constructs can be optionally further processed (e.g., to add any additional sequences and/or for clean-up) and subjected to sequencing. As described elsewhere herein, a sequence derived from the capture probe 606 spatial barcode sequence may be used to identify a feature and the sequence derived from spatial barcode sequence on the capture agent barcode domain 601 may be used to identify the particular peptide MHC complex 604 bound on the surface of the cell (e.g., when using MHC-peptide libraries for screening immune cells or immune cell populations).

Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a connected probe (e.g., a ligation product) or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form a connected probe (e.g., a ligation product) with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe.

In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a connected probe (e.g., a ligation product). In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the connected probe (e.g., a ligation product) is released from the analyte. In some instances, the connected probe (e.g., a ligation product) is released using an endonuclease (e.g., RNAse H). The released connected probe (e.g., a ligation product) can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### II. Spatial Cell-Based Analytical Methodology and Methods Involving Sorting Subsets of Nucleic Acids

Provided herein are methods for sorting subsets of nucleic acids from a biological sample into a cluster. For example, in some embodiments, such methods include contacting the biological sample with a plurality of capture probes, wherein a capture probe comprises a capture domain and a spatial barcode having a sequence; releasing nucleic acids from the biological sample, wherein members of the released nucleic acids are specifically bound by the capture domain(s); determining, for the nucleic acids that are specifically bound by the capture domain(s), (1) all or a portion of a sequence of the spatial barcode, or a complement thereof, and (2) all or a portion of a sequence of the nucleic acid or a complement thereof, and using the determined sequences of (1) and (2) to identify the location and amount of the nucleic acids in the biological sample; and comparing the determined location and amount of the nucleic acids at a plurality of different locations in the biological sample.

In some embodiments, methods of differentiating cell types in a biological sample are provided herein, e.g., the methods comprise sorting a subset of nucleic acids into a cluster based on the determined location and amount of the nucleic acids at the plurality of different locations in the biological sample, and using the cluster(s) to differentiate cell types in the biological sample. In some embodiments, methods of identifying a biological sample are provided herein, e.g., the methods comprise sorting a subset of nucleic acids into a cluster based on the determined location and amount of the nucleic acids at the plurality of different locations in the biological sample, and using the cluster(s) to identify the biological sample (e.g., the type of tissue the biological sample is from). In some embodiments, methods of generating an image of a biological sample are provided herein, e.g., the methods comprise sorting a subset of nucleic acids into a cluster based on the determined location and amount of the nucleic acids at the plurality of different locations in the biological sample, and using the cluster(s) to generate an image of the biological sample.

In some embodiments, methods of molecular heterogeneity in a biological sample, e.g., the methods comprise sorting a subset of nucleic acids into a cluster based on the determined location and amount of the nucleic acids at the plurality of different locations in the biological sample, and using the cluster(s) to identify molecular heterogeneity in the biological sample relative to a reference biological sample. In some embodiments, methods of identifying a subject as having abnormal gene expression in at least one tissue, e.g., sorting a subset of nucleic acids of into a cluster based on the determined location and amount of the nucleic acids at a plurality of different locations in the biological sample, and using the cluster(s) to identify at least one region in the biological sample with abnormal gene expression relative to a reference biological sample. In some embodiments, methods of identifying a subject as having a cellular anomaly are provided herein, e.g., the methods comprise sorting a subset of nucleic acids into a cluster based on the determined location and amount of the nucleic acids at the plurality of different locations in the biological sample, and using the cluster(s) to identify at least one cellular anomaly in the biological sample. In some embodiments, methods of assessing the efficacy of a treatment or therapy in a subject are provided herein, e.g., sorting a subset of nucleic acids of into a cluster based on the determined location and amount of the nucleic acids at the plurality of different locations in the biological sample, and using the cluster(s) to identify at least one region in the biological sample having restored gene expression.

In some embodiments, the amount of one or more nucleic acids falls outside a predetermined threshold. In some embodiments, the amount of one or more nucleic acids are elevated compared to the amount of a reference nucleic acid. In some embodiments, the amount of one or more nucleic acids are reduced compared to the amount of a reference nucleic acid.

In some embodiments, methods of comparing at least two biological samples are provided herein, e.g., the methods comprise sorting a subset of nucleic acids into a first set of clusters based on the determined location and amount of the nucleic acid sat the plurality of different locations in a first biological sample, sorting a subset of nucleic acids into a second set of clusters based on the determined location and amount of the nucleic acids at the plurality of different locations in a second biological sample; and comparing the first and second sets of clusters (i.e., the clusters from the first and second biological samples).

In some embodiments, the first biological sample is from the same subject as the second biological sample. In some embodiments, there is a period of time between acquiring the first biological sample and acquiring the second biological or subsequent samples from the subject. In some embodiments, the period of time is about 1 day to about five years, e.g., about 1 day to about 10 days, about 1 day to about 1 month, about 1 day to about 6 months, about 1 day to about 1 year, about 1 day to about 1.5 years, about 1 day to about 2 years, about 1 day to about 2 years, about 1 day to about 4 years, about 4 years to about 5 years, about 3 years to about 5 years, about 2 years to about 5 years, or about 1 year to about 5 years. For example, about 1.5 years to about 2 years, about 1 year to about 2 years, about 6 months to about 2 years, about 1 to about 3 years, or about 2 to about 4 years. In some embodiments, the period of time is about 1 month, about 6 months, about 1 year, about 2 years, about 3 years, about 4 years, or about 5 years. In some embodiments, the method further comprises comparing the clusters from additional biological samples obtained from the subject before and after the period of time.

In some embodiments, the first biological sample is obtained from a first subject and the second biological sample is obtained from a second subject. In some embodiments, the second biological sample is obtained from a healthy subject. In some embodiments, the first biological sample is obtained from a subject at risk (e.g., increased risk) of developing a disease.

In some embodiments, methods provided herein include sorting a subset of nucleic acids into a first set of clusters based on the determined amount and location of the nucleic acids at the plurality of different locations in the biological sample; and comparing the set of clusters to a reference set of clusters. In some embodiments, the reference set of clusters is a normalized set of clusters from more than one reference biological sample. In some embodiments, each of the more than one reference biological sample comprises the same type of tissue as the biological sample obtained from the subject.

In some embodiments, a method as described herein can further comprise identifying a subpopulation of cells in the biological sample.

In some embodiments, the biological sample comprises an epithelial tissue, a connective tissue, a muscle tissue, an adipose tissue, a nervous tissue, an embryonic tissue, or a combination thereof. In some embodiments, the biological sample comprises a brain tissue, a spinal cord tissue, a skin tissue, an adipose tissue, an intestinal tissue, a colon tissue, a cervical tissue, a vaginal tissue, a muscle tissue, a cardiac tissue, a liver tissue, a pancreatic tissue, a kidney tissue, a spleen tissue, a lymph node tissue, a bone marrow tissue, a cartilage tissue, a retinal tissue, a corneal tissue, a breast tissue, a prostate tissue, a bladder tissue, a tracheal tissue, a lung tissue, a uterine tissue, a stomach tissue, a thyroid tissue, a thymus tissue, or a combination thereof.

In some embodiments, the biological sample is obtained from a biopsy. In some embodiments, the biological sample is obtained from a surgical excision. In some embodiments, the biological sample was collected during an endoscopy or colposcopy.

(a) Reference Amounts

A reference amount of a nucleic acid/protein can be any appropriate reference amount. In some embodiments, a reference amount of a nucleic acid/protein can be determined based on an amount of the nucleic acid/protein in a corresponding sample (e.g., a reference sample such as a control subject not diagnosed with a disorder, not presenting with any of the symptoms of a disorder, not having a family history of a disorder, and not having any known risk factors of a disorder) at a corresponding position. In some embodiments, a reference amount of a nucleic acid/protein can be determined based on an amount of the nucleic acid/protein in one or more other locations in a sample. In some embodiments, a reference amount of a nucleic acid/protein can be a composite or averaged amount (e.g., the averaged amount of a population of persons having or not having a particular disorder).

In some embodiments, a reference amount can be based on a reference amount as published by an appropriate body (e.g., a government agency (e.g., the United States Food and Drug Administration) or a professional organization (e.g., the American Medical Association or American Psychiatric Association)), for example, a reference amount that is a threshold amount for a nucleic acid/protein at the location in the tissue of a subject.

In some embodiments, a reference amount of a nucleic acid/protein can be determined based on any appropriate criteria. For example, in some embodiments, a reference amount of a nucleic acid/protein can come from an age-matched healthy subject. In some embodiments, a reference amount of a nucleic acid/protein can come from a sex-matched healthy subject or a sex-matched healthy subject population. In some embodiments, a reference amount of a nucleic acid/protein can come from an age-matched, sex-matched healthy subject or an age-matched, sex-matched healthy subject population. In some embodiments, a reference amount of a nucleic acid/protein can come from an aggregate sample (e.g., an average of 2 or more individual) of healthy subjects (e.g., that are age-matched and/or sex-matched).

A healthy subject can be any appropriate healthy subject. In some embodiments, a healthy subject does not have the disorder of interest, does not have symptoms of the disorder, does not have a genetic mutation associated with the disorder of interest, does not have a family medical history of the disorder of interest, no behavior risk factors of the disorder of interest, or combinations thereof. For example, in some embodiments, a healthy subject has one or more of: no known brain disorder, no presentation of symptoms, or no more than three (e.g., no more than two, or no more than one) of: a brain disorder, no known genetic mutations associated with risk of a brain disorder, no family medical history of a brain disorder, and no behavioral risk factors of a brain disorder. Other non-limiting examples of healthy subjects are those that do not have a disorder of a biological system of interest (e.g., circulatory system, digestive and excretory system, endocrine system, integumentary or exocrine system, immune and lymphatic system, muscular system, nervous system, see the brain example above, renal and urinary system, reproductive system, respiratory system, skeletal system, or combinations thereof), does not have symptoms of the disorder, does not have a genetic mutation associated with the disorder of interest, does not have a family medical history of the disorder of interest, no behavior risk factors of the disorder of interest, or combinations thereof.

In some cases, an amount of a nucleic acid/protein can be elevated relative to a reference amount. For example, an amount of a nucleic acid/protein can be at least 0.2-fold (e.g., at least 0.4-fold, at least 0.6-fold, at least 0.8-fold, at least 1-fold, at least 1.3-fold, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, 30-fold, 40-fold, 50-fold, or more) greater than a reference amount (e.g., any of the exemplary reference amounts described herein or known in the art).

In some cases, an amount of a nucleic acid/protein can be decreased relative to a reference amount. For example, an amount of a nucleic acid/protein can be at least 0.2-fold (e.g., at least 0.4-fold, at least 0.6-fold, at least 0.8-fold, at least 1-fold, at least 1.3-fold, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, 30-fold, 40-fold, 50-fold, or more) less than a reference amount (e.g., any of the exemplary reference amounts described herein or known in the art).

In some cases, an amount of a nucleic acid/protein can be elevated relative to a reference amount. For example, an amount of a nucleic acid can be at least 5% more, at least 10% more, at least 15% more, at least 20% more, at least 25% more, at least 30% more, at least 35% more, at least 40% more, at least 45% more, at least 50% more, at least 55%, at least 60% more, at least 65% more, at least 70% more, at least 75% more, at least 80% more, at least 85% more, at least 90% more, at least 95% elevated (e.g., about a 5% to about a 99% increase, about a 5% increase to about a 80% increase, about a 5% increase to about a 60% increase, about a 5% increase to about a 40% increase, about a 5% increase to about a 20% increase, about a 20% increase to about a 95% increase, about a 20% increase to about a 80% increase, about a 20% increase to about a 60% increase, about a 20% increase to about a 40% increase, about a 40% increase to about a 99% increase, about a 40% increase to about a 80% increase, about a 40% increase to about a 60% increase, about a 60% increase to about a 99% increase, about a 60% increase to about a 80% increase, about a 80% increase to about a 99% increase) as compared to a reference amount (e.g., any of the exemplary reference amounts described herein).

In some cases, an amount of a nucleic acid can be decreased relative to a reference amount. For example, an amount of a nucleic acid/protein can be at least 5% less, at least 10% less, at least 15% less, at least 20% less, at least 25% less, at least 30% less, at least 35% less, at least 40% less, at least 45% less, at least 50% less, at least 55%, at least 60% less, at least 65% less, at least 70% less, at least 75% less, at least 80% less, at least 85% less, at least 90% less, at least 95% decreased (e.g., about a 5% to about a 99% decrease, about a 5% decrease to about a 80% decrease, about a 5% decrease to about a 60% decrease, about a 5% decrease to about a 40% decrease, about a 5% decrease to about a 20% decrease, about a 20% decrease to about a 95% decrease, about a 20% decrease to about a 80% decrease, about a 20% decrease to about a 60% decrease, about a 20% decrease to about a 40% decrease, about a 40% decrease to about a 99% decrease, about a 40% decrease to about a 80% decrease, about a 40% decrease to about a 60% decrease, about a 60% decrease to about a 99% decrease, about a 60% decrease to about a 80% decrease, about a 80% decrease to about a 99% decrease) as compared to a reference amount (e.g., any of the exemplary reference amounts described herein). Other suitable reference amounts and methods of determining the same will be apparent to those skilled in the field.

(b) Locations in a Sample

As used herein, a location in a sample can be any appropriate location. For example, in some embodiments, a location can be in one or more of a basal ganglia (e.g., a striatum, a caudate nucleus, a putamen, a nucleus accumbens, an olfactory tubercle, a globus pallidus, a ventral pallidum, substantia nigra, a subthanamic nucleus, or a combination or substructure or any thereof), a brain stem (e.g., a medulla oblongata, a midbrain, a pons, or a combination or substructure of any thereof), a cerebellum, a cerebral cortex (e.g., a lobe of a cerebral cortex, an isocortex, a cortical subplate, or a combination or substructure of any thereof), a limbic system (e.g., a prefrontal cortex (e.g., a cingulate gyrus, a thalamus, a hippocampus (e.g., a parahippocampal gyrus and/or a subiculum)), an amygdala, a nucleus accumbens, a hypothalamus, a ventral tegmental area, a raphe nuclei, a habenular commissure, an entorhinal cortex, an olfactory bulb(s), a medial forebrain bundle, and a piriform cortex.

In some embodiments, a location can be a lobe of a cerebral cortex (e.g., a frontal lobe, a parietal lobe, a temporal lobe, or an occipital lobe). In some embodiments, a location can be in a hypothalamus (or substructure thereof). In some embodiments, a location can be in a limbic system (or substructure thereof). In some embodiments, a location can be in a hippocampus (or substructure thereof). In some embodiments, a location can be in a cerebral cortex (or substructure thereof). In some embodiments, a location can be in a brain stem (or substructure thereof). In some embodiments, a location can be in a basal ganglia (or a substructure thereof). In some embodiments, a location can be in a substantia nigra (or a substructure thereof).

In some embodiments, a location can be in one or more of epithelial tissue, connective tissue, a muscle, adipose tissue, nervous tissue, and embryonic tissue. In some embodiments a location can be in one or more of a brain, a spinal cord, skin, adipose tissue, an intestine, a colon, a cervix, vaginal tissue, a muscle, a cardiac muscle, a liver, a pancreas, a kidney, a spleen, a lymph node, bone marrow, cartilage, a retina, a cornea, a breast, a prostate, a bladder, a trachea, a lung, a uterus, a stomach, a thyroid, and a thymus.

(c) Clusters

Many methods can be used to help identify a cluster. Non-limiting examples of such methods include nonlinear dimensionality reduction methods such as t-distributed stochastic neighbor embedding (t-SNE), global t-distributed stochastic neighbor embedding (g-SNE), and uniform manifold approximation and projection (UMAP).

Any number of clusters can be identified. In some embodiments, 2 to 500 clusters can be identified using the methods as described herein. For example, 2 to 10, 2 to 20, 2 to 50, 2 to 75, 2 to 100, 2 to 150, 2 to 200, 2 to 300, 2 to 400, 400 to 500, 300 to 500, 200 to 500, 100 to 500, 75 to 500, 50 to 500, or 25 to 200 clusters can be identified. In some embodiments, 25 to 75, 50 to 100, 50 to 150, 75 to 150, or 100 to 200 clusters can be identified.

Any number of nucleic acids can be sorted into a cluster. For example, a cluster can include about 1 to about 200,000 nucleic acids. In some embodiments, a cluster can include about 1 to about 150,000, about 1 to about 100,000, about 1 to about 75,000, about 1 to about 50,000, about 100,000 to about 200,000, or about 50,000 to about 200,000 nucleic acids. In some embodiments, a cluster includes about 2 to about 25,000 nucleic acids. For example, about 2 to about 50, about 2 to about 100, about 2 to about 500, about 2 to about 1,000, about 2 to about 5,000, about 2 to about 10,000, about 2 to about 15,000, about 2 to about 20,000, about 20,000 to about 25,000, about 15,000 to about 25,000, about 10,000 to about 25,000, about 5,000 to about 25,000, about 1,000 to about 25,000, about 500 to about 25,000, or about 100 to about 25,000 nucleic acids.

In some embodiments, a nucleic acid included in a cluster is different than each of the other nucleic acids in the cluster. For example, the nucleic acid has a sequence that is not identical to any of the other nucleic acids in the cluster. In some embodiments, a nucleic acid corresponds to a gene.

(d) Cancer

Pre-cancerous and cancerous cells can display genetic changes compared to non-cancerous cells. Furthermore, cancer can be a hetergenous disease, e.g., cancers can vary from patient to patient, and cells within the same tumor can even display heterogeneity. See, e.g., Allison and Sledge. Oncology (Williston Park). 28(9):772-8, 2014; which is incorporated by reference herein in its entirety. Additional genetic changes can occur in cancerous cells over time (see, e.g., Lipinski et al. Trends Cancer. 2(1):49-63, 2016; which is incorporated by reference herein in its entirety). Several factors including specific genetic changes, tumoral heterogeneity, and subclonal heterogeneity, can all affect prognosis and/or treatment outcomes in subjects. The methods provided herein can be used to diagnose or assess cancers in a subject. For example, the methods provided herein can be used to determine intratumoral heterogeneity in one or more tumor samples from the subject, e.g., a subset of nucleic acids, proteins, or other biomarkers, can be identified by any number of nonlinear dimensionality reduction techniques applied to the tumor sample dataset derived from detected biomarker amount and location at a plurality of different locations in the tumor sample. Generally, the nonlinear dimensionality reduction technique can identify cells sharing particular biomarkers or traits by clustering like cells together within an output or model, wherein the cluster(s) can be used to identify populations of cells that may not be visually identifiable. In the case of cancer, the cluster(s) can be used to identify whether a biological sample contains cancer cells. Further, the cluster(s) can be used to identify specific sub-populations of cancer cells within a tumor sample (e.g., intratumoral heterogeneity). Further, the cluster(s) can be used to determine the invasiveness of cancer. In some embodiments, comparison of tumor samples harvested at different time points can be used to determine whether cells within a tumor are changing over time.

In some embodiments, methods for identifying clusters disclosed herein (e.g., UMAP, t-distributed stochastic neighbor embedding (t-SNE) plot or a global t-distributed stochastic neighbor embedding (g-SNE) plot, etc.) can be used to visualize heterogeneity across samples. For example, overlap between clusters on a UMAP plot can be indicative of similar genetic expression. No or little overlap between clusters on a UMAP plot can indicate that those samples have no or little similar genetic expression. In some embodiments, no or little overlap of clusters on a UMAP plot between a cancerous sample from a subject and a non-cancerous sample from the same tissue of origin as the cancerous sample can indicate that the cancerous cells have acquired many genetic changes compared to noncancerous cells. In some embodiments, no or little overlap of clusters on a UMAP plot between a cancerous sample from a subject and a non-cancerous sample from the same tissue of origin as the cancerous sample can indicate that the cancer has progressed farther than a cancer with more overlap. In some embodiments, overlap between tumor cell clusters on a UMAP plot indicate the tumor cell populations are similar, with the closer the clusters the more similar the tumor cell populations. In some embodiments, overlap between tumor clusters may show how tumor cell populations are changing (e.g., one set of tumor cells gradually acquiring additional genetic or biomarker changes).

(e) Biomarkers and Candidate Biomarkers

As used herein, a biomarker can be any appropriate biomarker. In some embodiments, a biomarker can be a nucleic acid (e.g., genomic DNA (gDNA), mRNA, or rRNA (e.g., bacterial 16S rRNA)), a protein (e.g., an enzyme, a cell surface marker, a structural protein, a tumor suppressor, an antibody, a cytokine, a peptide hormone, or an identifiable fragment, precursor, or degradation product of any thereof), a lipoprotein, a fatty acid, a cell (e.g., a cell type, for example, in a location indicative of disease), or a small molecule (e.g., an enzymatic cofactor, a hormone (e.g., a steroid hormone or a eicosanoid hormone), or a metabolite). In some embodiments, a biomarker can include an alteration in a nucleic acid (e.g., an insertion, a deletion, a point mutation, and/or methylation), for example, relative to a wildtype or control nucleic acid. In some embodiments, a biomarker can include an alteration in a protein (e.g., an inserted amino acid, a deletion of an amino acid, an amino acid substitution, and/or a post-translational modification (e.g., presence, absence, or a change in, for example, acylation, isoprenylation, phosphorylation, glycosylation, methylation, hydroxylation, amidation, and/or ubiquitinylation)), for example, relative to a control or wildtype protein.

In some embodiments, a biomarker is a nucleic acid. In some embodiments, a biomarker is an mRNA. In some embodiments, a biomarker is a protein. In some embodiments, a biomarker is an enzyme. In some embodiments, a biomarker is a cell surface marker.

(f) Biomarkers of Glioblastoma

In some instances of the present disclosure, the term “biomarkers of glioblastoma” includes biomarkers of microglial cells. In some instances, the microglial cells are associated with glioblastoma. Microglia are innate immune cells in the central nervous system that make up a substantial portion of the tumor mass in gliomas, including glioblastomas (Abels, Erik R et al. (2019) Cell reports vol. 28, 12: 3105-3119). Glioblastomas are capable of interacting with microglia, which contributes to the growth of these tumors (Matias D et al., (2017) Reviews on Cancer, 1868(1): 333-340).

Non-limiting biomarkers of glioblastoma include COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, SLN, SRPX2, METTL7B, POSTN, NNMT, TIMP4, SERPINA3, KLHDC8A, NES, F2R, XIST, COL1A2, COL4A1, CA12, ANXA2, WWTR1, COL4A1, LAMB2, SPARC, FN1, TNFRSF1A, HLA-DRA, ALDH1L1, FLNA, NAMPT, VEGFA, C3, HLA-A, GRN, HLA-B, TPP1, HLA-B, HLA-DRA, LAMB2, and NAMPT.

Additional non-limiting biomarkers of glioblastoma include CD44, periostin (POSTN), nestin (NES), telomerase reverse transcriptase (TERT), uromodulin (UMOD), serum/glucocorticoid regulated kinase 1 (SGK1), G protein-coupled receptor 37 like 1 (GPR37L1), ISG15 ubiquitin like modifier (ISG15), and regulator of G protein signaling 5 (RGS5).

In some instances, additional non-limiting biomarkers include biomarkers of microglial cells. In some instances, the biomarkers are also associated with glioblastoma. In some instances, these biomarkers include one or more of proteolipid protein 1 (PLP1), chimerin 1 (CHN1), F-box and leucine rich repeat protein 16 (FBXL16), NSF attachment protein beta (NAPB), ATP synthase FO subunit 8 (MT-ATP8), maturin, neural progenitor differentiation regulator homolog (MTURN), N-terminal EF-hand calcium binding protein 1 (NECAB1), brain abundant membrane attached signal protein 1 (BASP1), RUN domain containing 3A (RUNDC3A), neurofilament medium (NEFM), phytanoyl-CoA 2-hydroxylase interacting protein (PHYHIP), RAB3A, member RAS oncogene family (RAB3A), ectodermal-neural cortex 1 (ENC1), transgelin 3 (TAGLN3), G protein subunit gamma 3 (GNG3), visinin like 1 (VSNL1), kinesin family member 1A (KIF1A), stathmin 2 (STMN2), ATPase Na+/K+ transporting subunit alpha 3 (ATP1A3), contactin 1 (CNTN1), eukaryotic translation elongation factor 1 alpha 2 (EEF1A2), neurogranin (NRGN), calcium binding protein 1 (CABP1), CUGBP Elav-like family member 4 (CELF4), calcyon neuron specific vesicular protein (CALY), synapsin II (SYN2), tubulin beta 4A class IVa (TUBB4A), myelin basic protein (MBP), synapsin I (SYN1), ATPase plasma membrane Ca2+ transporting 2 (ATP2B2), synaptosome associated protein 25 (SNAP25), gamma-aminobutyric acid type A receptor subunit alpha1 (GABRA1), solute carrier family 17 member 7 (SLC17A7), glutamate ionotropic receptor NMDA type subunit 1 (GRIN1), cholecystokinin (CCK), collagen type I alpha 1 chain (COL1A1), SPOC domain containing 1 (SPOCD1), WEE1 G2 checkpoint kinase (WEE1), serpin family E member 1 (SERPINE1), collagen type VIII alpha 1 chain (COL8A1), chitinase 3 like 1 (CI31), perilipin 2 (PLIN2), mitochondrial genome maintenance exonuclease 1 (DDK1), matrix G1a protein (MGP), annexin A1 (ANXA1), sushi repeat containing protein X-linked (SRPX), TIMP metallopeptidase inhibitor 1 (TIMP1), fibronectin 1 (FN1), secreted protein acidic and cysteine rich (SPARC), transgelin 2 (TAGLN2), cellular communication network factor 2 (CTGF), insulin like growth factor binding protein 7 (IGFBP7), nicotinamide phosphoribosyltransferase (NAMPT), caveolin 1 (CAV1), tenascin C (TNC), vascular endothelial growth factor A (VEGFA), adrenomedullin (ADM), CD44, insulin like growth factor binding protein 2 (IGFBP2), secreted phosphoprotein 1 (SPP1), 1,4-alpha-glucan branching enzyme 1 (GBE1), Y-box binding protein 3 (YBX3), vimentin (VIM), galectin 3 (LGALS3), small integral membrane protein 3 (SMIM3), chloride intracellular channel 1 (CLIC1), collagen type VI alpha 2 chain (COL6A2), podoplanin (PDPN), epithelial membrane protein 1 (EMP1), apolipoprotein C1 (APOC1), epithelial membrane protein 3 (EMP3), interferon induced transmembrane protein 2 (IFITM2), WW domain containing transcription regulator 1 (WWTR1), metallothionein 2A (MT2A), metallothionein 1× (MT1X), insulin like growth factor binding protein 3 (IGFBP3), cellular communication network factor 1 (CYR61), and insulin like growth factor binding protein 5 (IGFBP5).

In some instances, non-limiting biomarkers of glioblastoma include one or more of PLP1, CHN1, FBXL16, NAPB, MT-ATP8, MTURN, NECAB1, BASP1, RUNDC3A, NEFM, PHYHIP, RAB3A, ENC1, TAGLN3, GNG3, VSNL1, KIF1A, STMN2, ATP1A3, CNTN1, EEF1A2, NRGN, CABP1, CELF4, CALY, SYN2, TUBB4A, MBP, SYN1, ATP2B2, SNAP25, GABRA, SLC17A7, GRIN1, and CCK. In some embodiments, such biomarkers of glioblastoma are downregulated as compared to a reference level disclosed herein.

In some instances, non-limiting biomarkers of glioblastoma include one or more of COL1A1, SPOCD1, WEE1, SERPINE1, COL8A1, CHI3L1, PLIN2, DDK1, MGP, ANXA1, SRPX, TIMP1, FN1, SPARC, TAGLN2, CTGF, IGFBP7, NAMPT, CAV1, TNC, VEGFA, ADM, CD44, IGFBP2, SPP1, GBE1, YBX3, VIM, LGALS3, SMIM3, CLIC1, COL6A2, PDPN, EMP1, APOC1, EMP3, IFITM2, WWTR1, MT2A, MT1X, IGFBP3, CYR61, and IGFBP5. In some embodiments, such biomarkers of glioblastoma are upregulated as compared to a reference level disclosed herein

In some instances, non-limiting biomarkers of glioblastoma include one or more of NAPB, BASP1, RUNDC3A, NEFM, RAB3A, GNG3, KIF1A, ATP1A3, CNTN1, CELF4, SYN2, TUBB4A, GRIN1, SPOCD1, DDK1, TNC, GBE1, SMIM3, CLIC1, MT1X, and CYR61. In some instances, additional non-limiting biomarkers of glioblastoma include one or more of NAPB, BASP1, RUNDC3A, NEFM, RAB3A, GNG3, KIF1A, ATP1A3, CNTN1, CELF4, SYN2, TUBB4A, and GRIN1. In some instances, additional non-limiting biomarkers of glioblastoma include one or more of SPOCD1, DDK1, TNC, GBE1, SMIM3, CLIC1, MT1X, and CYR61. In some embodiments, levels of any one or more of these biomarkers are decreased as compared to a reference sample.

In some instances, additional non-limiting biomarkers of glioblastoma include biomarkers of microglia (i.e., co-expression of IBA1) associated with glioblastoma, including one or more of hemoglobin subunit alpha 2 (HBA2), hemoglobin subunit beta (HBB), hemoglobin subunit alpha 1 (HBA1), COL1A2, metastasis associated lung adenocarcinoma transcript 1 (MALAT1), RNA binding motif protein 25 (RBM25), solute carrier family 25 member 37 (SLC25A37), natural killer cell triggering receptor (NKTR), LUC7 like 3 pre-mRNA splicing factor (LUC7L3), ATP1A2, PNN interacting serine and arginine rich protein (PNISR), maternally expressed 3 (MEG3), interferon induced protein 44 like (IFI44L), family with sequence similarity 133 member B (FAM133B), pinin, desmosome associated protein (PNN), pleckstrin homology domain containing A4 (PLEKHA4), parathymosin (PTMS), B double prime 1, subunit of RNA polymerase III transcription initiation factor IIIB (BDP1), MTRNR2L12, splicing regulatory glutamic acid and lysine rich protein 1 (SREK1), arginine and glutamate rich 1 (ARGLU1), XIAP associated factor 1 (XAF1), MT-RNR2 like 8 (MTRNR2L8), serine/arginine repetitive matrix 2 (SRRM2), COL4A1, dickkopf WNT signaling pathway inhibitor 1 (DKK1), CHI3L1, heparan sulfate 2-O-sulfotransferase 1 (HS2ST1), early growth response 1 (EGR1), transcriptional and immune response regulator (TCIM), PLIN2, APOC1, Fos proto-oncogene, AP-1 transcription factor subunit (FOS), MGP, SPP1, ribosomal protein L17 (RPL17), TNC, interferon induced transmembrane protein 3 (IFITM3), MT2A, thymosin beta 4 X-linked (TMSB4X), thymosin beta 10 (TMSB10), PDPN, cytochrome c oxidase subunit 6C (COX6C), VIM, chloride intracellular channel 1 (CLIC1), IFITM2, transcription elongation factor A like 9 (TCEAL9), ribosomal protein L12 (RPL12), TAGLN, and NAMPT.

In some instances, non-limiting biomarkers of glioblastoma (e.g., biomarkers of microglia associated with glioblastoma) that are downregulated include one or more of HBA2, HBB, HBA1, COL1A2, MALAT1, RBM25, SLC25A37, NKTR, LUC7L3, ATP1A2, PNISR, MEG3, IFI44L, FAM133B, PNN, PLEKHA4, PTMS, BDP1, MTRNR2L12, SREK1, ARGLU1, XAF1, MTRNR2L8, SRRM2, and COL4A1.

In some instances, non-limiting biomarkers of glioblastoma (e.g., biomarkers of microglia associated with glioblastoma) that are upregulated include one or more of DKK1, CHI3L1, HS2ST1, EGR1, TCIM, PLIN2, APOC1, FOS, MGP, SPP1, RPL17, TNC, IFITM3, MT2A, TMSB4X, TMSB10, PDPN, COX6C, VIM, CLIC1, IFITM2, TCEAL9, RPL12, TAGLN, and NAMPT.

In some instances, non-limiting biomarkers of glioblastoma include one or more of HBA2, HBB, HBA1, MALAT1, RBM25, SLC25A37, NKTR, LUC7L3, PNISR, MEG3, IFI44L, FAM133B, PNN, PLEKHA4, PTMS, BDP1, MTRNR2L12, SREK1, ARGLU1, XAF1, MTRNR2L8, SRRM2, COL4A1, DKK1, HS2ST1, EGR1, TCIM, FOS, RPL17, TNC, IFITM3, TMSB4X, TMSB10, COX6C, CLIC1, TCEAL9, RPL12, TAGLN, AND NAMPT. In some instances, non-limiting biomarkers of glioblastoma include one or more of HBA2, HBB, HBA1, MALAT1, RBM25, SLC25A37, NKTR, LUC7L3, PNISR, MEG3, IFI44L, FAM133B, PNN, PLEKHA4, PTMS, BDP1, MTRNR2L12, SREK1, ARGLU1, XAF1, MTRNR2L8, SRRM2, and COL4A1. In some instances, non-limiting biomarkers of glioblastoma include one or more ofDKK1, HS2ST1, EGR1, TCIM, FOS, RPL17, TNC, IFITM3, TMSB4X, TMSB10, COX6C, CLIC1, TCEAL9, RPL12, TAGLN, and NAMPT.

TABLE 1
Exemplary Biomarkers of Glioblastoma

| | Protein PubMed | cDNA PubMed |
|---|---|---|
| Biomarker | Accession No. | Accession No. |
| COL1A1 | NP_000079.2 | NM_000088.4 |
| COL3A1 | NP_000081.2 | NM_000090.4 |
| COL8A1 | NP_065084.2 | NM_020351 |
| WEE1 | NP_003381.1 | NM_003390.4 |
| CHI3L1 | NP_001267.2 | NM_001276.4 |
| MGP | NP_000891.2 | NM_000900.5 |
| SRPX | NP_006298.1 | NM_006307.5 |
| SERPINE1 | NP_000593.1 | NM_000602.5 |
| COL1A2 | NP_000080.2 | NM_000089.4 |
| TIMP1 | NP_003245.1 | NM_003254.3 |
| ANXA1 | NP_000691.1 | NM_000700.3 |
| COL6A2 | NP_001840.3 | NM_001849.4 |
| CAV1 | NP_001744.2 | NM_001753.5 |
| PLIN2 | NP_001113.2 | NM_001122.4 |
| CD44 | NP_000601.3 | NM_000610.4 |
| APOC1 | NP_001636.1 | NM_001645.5 |
| IGFBP2 | NP_000588.3 | NM_000597.3 |
| PDPN | NP_006465.3 | NM_006474.4 |
| VIM | NP_003371.2 | NM_003380.5 |
| LGALS3 | NP_002297.2 | NM_002306.4 |
| VEGFA | NP_003367.4 | NM_003376.6 |
| IGFBP5 | NP_000590.1 | NM_000599.4 |
| CTGF | NP_001892.1 | NM_001901.3 |
| EMP1 | NP_001414.1 | NM_001423.3 |
| EMP3 | NP_001416.1 | NM_001425.3 |
| IGFBP3 | NP_001013416.1 | NM_001013398.2 |
| A2M | NP_000005.3 | NM_000014.6 |
| ANXA2 | NP_004030.1 | NM_004039.3 |
| FLNA | NP_001104026.1 | NM_001110556.2 |
| IFGBP7 | NP_001544.1 | NM_001553.3 |
| S100A11 | NP_005611.1 | NM_005620.2 |
| ADM | NP_001115.1 | NM_001124.3 |
| FN1 | NP_997647.1 | NM_212482.3 |
| SERPING1 | NP_000053.2 | NM_000062.3 |
| MT2A | NP_005944.1 | NM_005953.5 |
| S100A10 | NP_002957.1 | NM_002966.3 |
| SPARC | NP_003109.1 | NM_003118.4 |
| ITGB1 | NP_002202.2 | NM_002211.4 |
| SLC5A3 | NP_008864.4 | NM_006933.7 |
| FABP7 | NP_001437.1 | NM_001446.5 |
| YBX3 | NP_003642.3 | NM_003651.5 |
| IFITM2 | NP_006426.2 | NM_006435.2 |
| TAGLN2 | NP_003555.1 | NM_003564.3 |
| COL6A1 | NP_001839.2 | NM_001848.3 |
| HLA-A | NP_002107.3 | NM_002116.8 |
| LGALS3BP | NP_005558.1 | NM_005567.4 |
| ANXA5 | NP_001145.1 | NM_001154.4 |
| APOE | NP_000032.1 | NM_000041.4 |
| GADD45A | NP_001915.1 | NM_001924.4 |
| TPM4 | NP_003281.1 | NM_003290.3 |
| SPP1 | NP_001035147.1 | NM_001040058.2 |
| GABRA1 | NP_001121116.1 | NM_001127644.2 |
| CCK | NP_000720.1 | NM_000729.6 |
| SLC17A7 | NP_064705.1 | NM_020309.4 |
| CHGA | NP_001266.1 | NM_001275.4 |
| STMN2 | NP_008960.2 | NM_007029.4 |
| CALY | NP_056537.1 | NM_015722.4 |
| EEF1A2 | NP_001949.1 | NM_001958.5 |
| CABP1 | NP_001028849.1 | NM_001033677.1 |
| NRGN | NP_006167.1 | NM_006176.3 |
| SNAP25 | NP_570824.1 | NM_130811.4 |
| ATP2B2 | NP_001001331.1 | NM_001001331.4 |
| SYN1 | NP_008881.2 | NM_006950.3 |
| NECAB1 | NP_071746.1 | NM_022351.5 |
| MBP | NP_001020272.1 | NM_001025101.2 |
| PHYHIP | NP_055574.3 | NM_014759.5 |
| BASP | NP_006308.3 | NM_006317.5 |
| CPLX1 | NP_006642.1 | NM_006651.4 |
| VSNL1 | NP_003376.2 | NM_003385.5 |
| TAGLN3 | NP_001008273.1 | NM_001008272.2 |
| ENC1 | NP_003624.1 | NM_003633.3 |
| FBXL16 | NP_699181.2 | NM_153350.4 |
| CHN1 | NP_001813.1 | NM_001822.7 |
| KIF5A | NP_004975.2 | NM_004984.4 |
| PLP1 | NP_000524.3 | NM_000533.5 |
| OLFM1 | NP_001269540.1 | NM_001282611.2 |
| SNCB | NP_003076.1 | NM_003085.5 |
| STXBP1 | NP_001027392.1 | NM_001032221.6 |
| ATP1B1 | NP_001668.1 | NM_001677.4 |
| DNM1 | NP_004399.2 | NM_004408.4 |
| SERPINI1 | NP_001116224.1 | NM_001122752.1 |
| PRKAR1B | NP_001158232.1 | NM_001164760.2 |
| MEF2C | NP_002388.2 | NM_002397.5 |
| MTURN | NP_690006.2 | NM_152793.3 |
| NSF | NP_006169.2 | NM_006178.4 |
| SYT1 | NP_005630.1 | NM_005639.3 |
| MAP2 | NP_001362434.1 | NM_001375505.1 |
| MT-ATP8 | YP_003024030.1 | |
| MAP1A | NP_002364.5 | NM_002373.6 |
| UCHL1 | NP_004172.2 | NM_004181.5 |
| FAIM2 | NP_036438.2 | NM_012306.4 |
| STMN1 | NP_005554.1 | NM_005563.4 |
| APLP1 | NP_001019978.1 | NM_001024807.3 |
| NCDN | NP_055099.1 | NM_014284.3 |
| STMN3 | NP_056978.2 | NM_015894.4 |
| MT-ND4L | YP_003024034.1 | |
| BEX1 | NP_060946.3 | NM_018476.4 |
| MT-ND2 | YP_003024027.1 | |
| PPP3CA | NP_000935.1 | NM_000944.5 |
| CPLX2 | NP_006641.1 | NM_006650.4 |
| ST8SIA3 | NP_056963.2 | NM_015879.3 |
| GABRG2 | NP_944494.1 | NM_198904.3 |
| KCNC2 | NP_631875.1 | NM_139137.4 |
| MT-ND5 | YP_003024036.1 | |
| CD44 | NP_000601.3 | NM_000610.4 |
| POSTN | NP_001129406.1 | NM_001135934.2 |
| NES | NP_006608.1 | NM_006617.2 |
| TERT | NP_001180305.1 | NM_001193376.3 |
| | NP_937983.2 | NM_198253.3 |
| UMOD | NP_001008390.1 | NM_001008389.3 |
| SGK1 | NP_001137148.1 | NM_001143676.2 |
| GPR37L1 | NP_004758.3 | NM_004767.5 |
| ISG15 | NP_005092.1 | NM_005101.4 |
| RGS5 | NP_001182232.1 | NM_001195303.3 |
| NAPB | NP_001269947.1 | NM_001283018.2 |
| BASP1 | NP_001258535.1 | NM_001271606.2 |
| RUNDC3A | NP_001138297.1 | NM_001144825.2 |
| NEFM | NP_001099011.1 | NM_001105541.2 |
| RAB3A | NP_002857.1 | NM_002866.5 |
| GNG3 | NP_036334.1 | NM_012202.5 |
| KIF1A | NP_001230937.1 | NM_001244008.2 |
| ATP1A3 | NP_001243142.1 | NM_001256213.2 |
| CNTN1 | NP_001242992.1 | NM_001256063.2 |
| CELF4 | NP_001020258.1 | NM_001025087.2 |
| SYN2 | NP_003169.2 | NM_003178.6 |
| TUBB4A | NP_001276052.1 | NM_001289123.2 |
| GRIN1 | NP_000823.4 | NM_000832.7 |
| SPOCD1 | NP_001268916.1 | NM_001281987.2 |
| DDK1 | NP_001297267.1 | NM_001310338.2 |
| TNC | NP_002151.2 | NM_002160.4 |
| GBE1 | NP_000149.4 | NM_000158.4 |
| SMIM3 | NP_116565.3 | NM_032947.5 |
| CLIC1 | NP_001274522.1 | NM_001287593.1 |
| MT1X | NP_005943.1 | NM_005952.4 |
| CYR61 | NP_001545.2 | NM_001554.5 |
| HBA2 | NP_000508.1 | NM_000517.6 |
| HBB | NP_000509.1 | NM_000518.5 |
| HBA1 | NP_000549.1 | NM_000558.5 |
| MALAT1 | | NR_002819.4 |
| RBM25 | NP_067062.1 | NM_021239.3 |
| SLC25A37 | NP_001304741.1 | NM_001317812.2 |
| NKTR | NP_001336053.1 | NM_001349124.2 |
| LUC7L3 | NP_001317259.1 | NM_001330330.2 |
| PNISR | NP_001309334.1 | NM_001322405.2 |
| IFI44L | NP_001362575.1 | NM_001375646.1 |
| FAM133B | NP_001035146.1 | NM_001040057.3 |
| PNN | NP_002678.3 | NM_002687.4 |
| PLEKHA4 | NP_001154826.1 | NM_001161354.2 |
| PTMS | NP_001317262.1 | NM_001330333.2 |
| BDP1 | NP_060899.2 | NM_018429.3 |
| SREK1 | NP_001070667.1 | NM_001077199.3 |
| ARGLU1 | NP_060481.3 | NM_018011.4 |
| XAF1 | NP_001340063.1 | NM_001353134.2 |
| MTRNR2L8 | NP_001177631.1 | NM_001190702.2 |
| SRRM2 | NP_057417.3 | NM_016333.4 |
| DKK1 | NP_036374.1 | NM_012242.4 |
| HS2ST1 | NP_001127964.1 | NM_001134492.2 |
| EGR1 | NP_001955.1 | NM_001964.3 |
| TCIM | NP_064515.2 | NM_020130.5 |
| FOS | NP_005243.1 | NM_005252.4 |
| RPL17 | NP_000976.1 | NM_000985.5 |
| IFITM3 | NP_066362.2 | NM_021034.3 |
| TMSB4X | NP_066932.1 | NM_021109.4 |
| TMSB10 | NP_066926.1 | NM_021103.4 |
| COX6C | NP_004365.1 | NM_004374.4 |
| CLIC1 | NP_001274522.1 | NM_001287593.1 |
| TCEAL9 | NP_001006613.1 | NM_001006612.2 |
| RPL12 | NP_000967.1 | NM_000976.4 |

Some embodiments of any of the methods described herein can include the detection of a level of one or more of COLA, COUA1, COL8A1, WEE, CH3L1, MGP, SRPX, SERPINE1, COL1A2, TIMIP1, ANXA1, COL6A2, CAV1, PLIN42, CD44, APOC1, IGFBP2, PDPN, VTM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APO, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, SLN, SRPX2, METTL7B, POSTN, NNMT, TIMP4, SERPINA3, KLHDC8A, NES, F2R, XIST, COL1A2, COL4A1, CA12, ANXA2, WWTR1, COL4A1, LAMB2, SPARC, FN1, TNFRSF1A, HLA-DRA, ALDH1L1, FLNA, NAMPT, VEGFA, C3, HLA-A, GRN, HLA-B, TPP1, HLA-B, HLA-DRA, LAMB2, and NAMPT or a byproduct, a degradation product, or a precursor thereof.

Some embodiments of any of the methods described herein can include the detection of a level of one or more of CD44, POSTN, NES, TERT, UMOD, SGK1, GPR37L1, ISG15, and RGS5, or a byproduct, a degradation product, or a precursor thereof.

Some embodiments of any of the methods described herein can include the detection of a level of one or more of PLP1, CHN1, FBXL16, NAPB, MT-ATP8, MTURN, NECAB1, BASP1, RUNDC3A, NEFM, PHYHIP, RAB3A, ENC1, TAGLN3, GNG3, VSNL1, KIF1A, STMN2, ATP1A3, CNTN1, EEF1A2, NRGN, CABP1, CELF4, CALY, SYN2, TUBB4A, MBP, SYN1, ATP2B2, SNAP25, GABRA1, SLC17A7, GRIN1, and CCK.

Some embodiments of any of the methods described herein can include the detection of a level of one or more of COL1A1, SPOCD1, WEE1, SERPINE1, COL8A1, CHI3L1, PLIN2, DDK1, MGP, ANXA1, SRPX, TIMP1, FN1, SPARC, TAGLN2, CTGF, IGFBP7, NAMPT, CAV1, TNC, VEGFA, ADM, CD44, IGFBP2, SPP1, GBE1, YBX3, VIM, LGALS3, SMIM3, CLIC1, COL6A2, PDPN, EMP1, APOC1, EMP3, IFITM2, WWTR1, MT2A, MT1X, IGFBP3, CYR61, and IGFBP5.

Some embodiments of any of the methods described herein can include the detection of a level of one or more of NAPB, BASP1, RUNDC3A, NEFM, RAB3A, GNG3, KIF1A, ATP1A3, CNTN1, CELF4, SYN2, TUBB4A, GRIN1, SPOCD1, DDK1, TNC, GBE1, SMIM3, CLIC1, MT1X, and CYR61.

Some embodiments of any of the methods described herein can include the detection of a level of one or more of HBA2, HBB, HBA1, COL1A2, MALAT1, RBM25, SLC25A37, NKTR, LUC7L3, ATP1A2, PNISR, MEG3, IFI44L, FAM133B, PNN, PLEKHA4, PTMS, BDP1, MTRNR2L12, SREK1, ARGLU1, XAF1, MTRNR2L8, SRRM2, COL4A1, or a byproduct, a degradation product, or a precursor thereof. Some embodiments of any of the methods described herein can include the detection of a level of one or more of DKK1, CHI3L1, HS2ST1, EGR1, TCIM, PLIN2, APOC1, FOS, MGP, SPP1, RPL17, TNC, IFITM3, MT2A, TMSB4X, TMSB10, PDPN, COX6C, VIM, CLIC1, IFITM2, TCEAL9, RPL12, TAGLN, NAMPT, or a byproduct, a degradation product, or a precursor thereof.

Some embodiments of any of the methods described herein can include the detection of a level of one or more of HBA2, HBB, HBA1, MALAT1, RBM25, SLC25A37, NKTR, LUC7L3, PNISR, MEG3, IFI44L, FAM133B, PNN, PLEKHA4, PTMS, BDP1, MTRNR2L12, SREK1, ARGLU1, XAF1, MTRNR2L8, SRRM2, DKK1, HS2ST1, EGR1, TCIM, FOS, RPL17, TNC, IFITM3, TMSB4X, TMSB10, COX6C, CLIC1, TCEAL9, RPL12, or a byproduct, a degradation product, or a precursor thereof. Some embodiments of any of the methods described herein can include the detection of a level of one or more of HBA2, HBB, HBA1, MALAT1, RBM25, SLC25A37, NKTR, LUC7L3, PNISR, MEG3, IFI44L, FAM133B, PNN, PLEKHA4, PTMS, BDP1, MTRNR2L12, SREK1, ARGLU1, XAF1, MTRNR2L8, SRRM2, or a byproduct, a degradation product, or a precursor thereof. Some embodiments of any of the methods described herein can include the detection of a level of one or more of DKK1, HS2ST1, EGR1, TCIM, FOS, RPL17, TNC, IFITM3, TMSB4X, TMSB10, COX6C, CLIC1, TCEAL9, RPL12, or a byproduct, a degradation product, or a precursor thereof.

(g) Methods of Detecting Biomarker(s) in a Location in a Sample

Any of the exemplary methods described herein can be used to determine a level and/or at least one activity of one or more biomarkers (e.g., one or more of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, CD44, POSTN, NES, TERT, UMOD, SGK1, GPR37L1, ISG15, RGS5, SLN, SRPX2, METTL7B, POSTN, NNMT, TIMP4, SERPINA3, KLHDC8A, NES, F2R, XIST, COL1A2, COL4A1, CA12, ANXA2, WWTR1, COL4A1, LAMB2, SPARC, FN1, TNFRSF1A, HLA-DRA, ALDH1L1, FLNA, NAMPT, VEGFA, C3, HLA-A, GRN, HLA-B, TPP1, HLA-B, HLA-DRA, LAMB2, NAMPT, NAPB, BASP1, RUNDC3A, NEFM, RAB3A, GNG3, KIF1A, ATP1A3, CNTN1, CELF4, SYN2, TUBB4A, GRIN1, SPOCD1, DDK1, TNC, GBE1, SMIM3, CLIC1, MT1X, CYR61, HBA2, HBB, HBA1, MALAT1, RBM25, SLC25A37, NKTR, LUC7L3, PNISR, MEG3, IFI44L, FAM133B, PNN, PLEKHA4, PTMS, BDP1, MTRNR2L12, SREK1, ARGLU1, XAF1, MTRNR2L8, SRRM2, DKK1, HS2ST1, EGR1, TCIM, FOS, RPL17, TNC, IFITM3, TMSB4X, TMSB10, COX6C, CLIC1, TCEAL9, and RPL12, or a byproduct, degradation product, or fragment, or precursor thereof) in a sample (e.g., a brain tissue sample or cerebrospinal fluid) or at a location in a sample (e.g., a brain tissue sample). In some embodiments, determining a level and/or an activity of one or more biomarkers (e.g., one or more of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, CD44, POSTN, NES, TERT, UMOD, SGK1, GPR37L1, ISG15, RGS5, SLN, SRPX2, METTL7B, POSTN, NNMT, TIMP4, SERPINA3, KLHDC8A, NES, F2R, XIST, COL1A2, COL4A1, CA12, ANXA2, WWTR1, COL4A1, LAMB2, SPARC, FN1, TNFRSF1A, HLA-DRA, ALDH1L1, FLNA, NAMPT, VEGFA, C3, HLA-A, GRN, HLA-B, TPP1, HLA-B, HLA-DRA, LAMB2, NAMPT NAPB, BASP1, RUNDC3A, NEFM, RAB3A, GNG3, KIF1A, ATP1A3, CNTN1, CELF4, SYN2, TUBB4A, GRIN1, SPOCD1, DDK1, TNC, GBE1, SMIM3, CLIC1, MT1X, CYR61, HBA2, HBB, HBA1, MALAT1, RBM25, SLC25A37, NKTR, LUC7L3, PNISR, MEG3, IFI44L, FAM133B, PNN, PLEKHA4, PTMS, BDP1, MTRNR2L12, SREK1, ARGLU1, XAF1, MTRNR2L8, SRRM2, DKK1, HS2ST1, EGR1, TCIM, FOS, RPL17, TNC, IFITM3, TMSB4X, TMSB10, COX6C, CLIC1, TCEAL9, and RPL12) can include any of the workflows described herein.

In some embodiments, the methods can include contacting the sample with a binding agent that specifically binds to a biomarker (e.g., one of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, CD44, POSTN, NES, TERT, UMOD, SGK1, GPR37L1, ISG15, RGS5, NAPB, BASP1, RUNDC3A, NEFM, RAB3A, GNG3, KIF1A, ATP1A3, CNTN1, CELF4, SYN2, TUBB4A, GRIN1, SPOCD1, DDK1, TNC, GBE1, SMIM3, CLIC1, MT1X, CYR61, HBA2, HBB, HBA1, MALAT1, RBM25, SLC25A37, NKTR, LUC7L3, PNISR, MEG3, IFI44L, FAM133B, PNN, PLEKHA4, PTMS, BDP1, MTRNR2L12, SREK1, ARGLU1, XAF1, MTRNR2L8, SRRM2, DKK1, HS2ST1, EGR1, TCIM, FOS, RPL17, TNC, IFITM3, TMSB4X, TMSB10, COX6C, CLIC1, TCEAL9, or RPL12, or a byproduct, degradation product, or fragment, or precursor thereof) (e.g., gDNA, mRNA, a protein, or a byproduct, degradation product, or fragment, or precursor thereof), wherein the binding agent further comprises an oligonucleotide having a sequence; and sequencing all or a portion of the sequence of the oligonucleotide or a complement thereof, from a probe specifically bound to the biomarker (e.g., one of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, CD44, POSTN, NES, TERT, UMOD, SGK1, GPR37L1, ISG15, RGS5, NAPB, BASP1, RUNDC3A, NEFM, RAB3A, GNG3, KIF1A, ATP1A3, CNTN1, CELF4, SYN2, TUBB4A, GRIN1, SPOCD1, DDK1, TNC, GBE1, SMIM3, CLIC1, MT1X, CYR61, HBA2, HBB, HBA1, MALAT1, RBM25, SLC25A37, NKTR, LUC7L3, PNISR, MEG3, IFI44L, FAM133B, PNN, PLEKHA4, PTMS, BDP1, MTRNR2L12, SREK1, ARGLU1, XAF1, MTRNR2L8, SRRM2, DKK1, HS2ST1, EGR1, TCIM, FOS, RPL17, TNC, IFITM3, TMSB4X, TMSB10, COX6C, CLIC1, TCEAL9, or RPL12, or a byproduct, degradation product, or fragment, or precursor thereof), to determine the level of the biomarker (e.g., COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, CD44, POSTN, NES, TERT, UMOD, SGK1, GPR37L1, ISG15, RGS5, NAPB, BASP1, RUNDC3A, NEFM, RAB3A, GNG3, KIF1A, ATP1A3, CNTN1, CELF4, SYN2, TUBB4A, GRIN1, SPOCD1, DDK1, TNC, GBE1, SMIM3, CLIC1, MT1X, CYR61, HBA2, HBB, HBA1, MALAT1, RBM25, SLC25A37, NKTR, LUC7L3, PNISR, MEG3, IFI44L, FAM133B, PNN, PLEKHA4, PTMS, BDP1, MTRNR2L12, SREK1, ARGLU1, XAF1, MTRNR2L8, SRRM2, DKK1, HS2ST1, EGR1, TCIM, FOS, RPL17, TNC, IFITM3, TMSB4X, TMSB10, COX6C, CLIC1, TCEAL9, or RPL12, or a byproduct, degradation product, or fragment, or precursor thereof) in the sample (e.g., cerebrospinal fluid or brain tissue) or at a location in the sample (e.g., brain tissue).

In some embodiments, the methods can include delivering a plurality of probes to a sample (e.g., a tissue sample, for instance, affixed to a support), wherein a probe of the plurality of probes includes a protein that specifically binds to a biomarker (e.g., one of COL1A1, COL3A1, COL8A1, WEE1, CHI3L1, MGP, SRPX, SERPINE1, COL1A2, TIMIP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, CD44, POSTN, NES, TERT, UMOD, SGK1, GPR37L1, ISG15, RGS5, NAPB, BASP1, RUNDC3A, NEFM, RAB3A, GNG3, KIF1A, ATP1A3, CNTN1, CELF4, SYN2, TUBB4A, GRIN1, SPOCD1, DDK1, TNC, GBE1, SMIM3, CLIC1, MT1X, CYR61, HBA2, HBB, HBA1, MALAT1, RBM25, SLC25A37, NKTR, LUC7L3, PNISR, MEG3, IFI44L, FAM133B, PNN, PLEKHA4, PTMS, BDP1, MTRNR2L12, SREK1, ARGLU1, XAF1, MTRNR2L8, SRRM2, DKK1, HS2ST1, EGR1, TCIM, FOS, RPL17, TNC, IFITM3, TMSB4X, TMSB10, COX6C, CLIC1, TCEAL9, RPL12, or a byproduct, degradation product, or fragment, or precursor thereof) (e.g., a protein, or a byproduct, degradation product, or fragment, or precursor thereof) in the tissue sample, wherein the protein is conjugated to an oligonucleotide having a sequence, and separating the probe specifically bound to the biomarker at the location of the tissue sample from the plurality of probes not specifically bound to the biomarker at the location of the tissue sample; and sequencing all or a portion of the sequence of the oligonucleotide or a complement thereof, from the specifically bound probe, and using the determined sequence to determine the level of the biomarker in a sample (e.g., cerebrospinal fluid or brain tissue) or to associate presence or abundance of the biomarker with the location of the tissue sample (e.g., brain tissue).

In some embodiments, the methods can include delivering a plurality of probes to a sample (e.g., a tissue sample, for instance, affixed to a support), wherein a probe of the plurality of probes includes a first oligonucleotide that specifically binds to a biomarker (e.g., one of COL1A1, COL3A1, COL8A1, WEE1, CHI3L1, MGP, SRPX, SERPINE1, COL1A2, TIMIP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, CD44, POSTN, NES, TERT, UMOD, SGK1, GPR37L1, ISG15, RGS5, NAPB, BASP1, RUNDC3A, NEFM, RAB3A, GNG3, KIF1A, ATP1A3, CNTN1, CELF4, SYN2, TUBB4A, GRIN1, SPOCD1, DDK1, TNC, GBE1, SMIM3, CLIC1, MT1X, CYR61, HBA2, HBB, HBA1, MALAT1, RBM25, SLC25A37, NKTR, LUC7L3, PNISR, MEG3, IFI44L, FAM133B, PNN, PLEKHA4, PTMS, BDP1, MTRNR2L12, SREK1, ARGLU1, XAF1, MTRNR2L8, SRRM2, DKK1, HS2ST1, EGR1, TCIM, FOS, RPL17, TNC, IFITM3, TMSB4X, TMSB10, COX6C, CLIC1, TCEAL9, or RPL12, or a byproduct, degradation product, or fragment, or precursor thereof) (e.g., gDNA, mRNA, or a byproduct, degradation product, or fragment, or precursor thereof) in the tissue sample, wherein the first oligonucleotide is conjugated to a second oligonucleotide having a sequence, and separating the probe specifically bound to the biomarker at the location of the tissue sample from the plurality of probes not specifically bound to the biomarker at the location of the tissue sample; and sequencing all or a portion of the sequence of the second oligonucleotide or a complement thereof, from the specifically bound probe, and using the determined sequence to determine the presence or level of the biomarker in the sample (e.g., brain tissue or cerebrospinal fluid) or to determine the presence or level of the biomarker at the location in the sample (e.g., brain tissue).

In some embodiments, the methods can include delivering a plurality of probes to a tissue sample, wherein at least one probe of the plurality of probes comprises a protein that specifically binds to a biomarker (e.g., one of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, CD44, POSTN, NES, TERT, UMOD, SGK1, GPR37L1, ISG15, RGS5, NAPB, BASP1, RUNDC3A, NEFM, RAB3A, GNG3, KIF1A, ATP1A3, CNTN1, CELF4, SYN2, TUBB4A, GRIN1, SPOCD1, DDK1, TNC, GBE1, SMIM3, CLIC1, MT1X, CYR61, HBA2, HBB, HBA1, MALAT1, RBM25, SLC25A37, NKTR, LUC7L3, PNISR, MEG3, IFI44L, FAM133B, PNN, PLEKHA4, PTMS, BDP1, MTRNR2L12, SREK1, ARGLU1, XAF1, MTRNR2L8, SRRM2, DKK1, HS2ST1, EGR1, TCIM, FOS, RPL17, TNC, IFITM3, TMSB4X, TMSB10, COX6C, CLIC1, TCEAL9, and RPL12, or a byproduct, degradation product, or fragment, or precursor thereof) (e.g., a protein, or a byproduct, degradation product, precursor, or fragment of any thereof) in the tissue sample, wherein the protein is conjugated to an oligonucleotide having a sequence, and wherein (i) each of the at least one probe comprises a protein that specifically binds a different biomarker of the tissue sample, and (ii) the protein of each of the at least one probe is conjugated to a different oligonucleotide having a sequence; imaging the tissue sample to identify a location of interest of the tissue sample; and sequencing all or a portion of the sequence(s) of the oligonucleotide(s) or a complement thereof, from the at least one probe specifically bound to the biomarker in the location of interest of the tissue sample, and using the determined sequence(s) to determine the presence or level of the biomarker in the sample (e.g., brain tissue or cerebrospinal fluid) or to determine the presence or level of the biomarker at the location in the sample (e.g., brain tissue).

In some embodiments, the methods can include delivering a plurality of probes to a tissue sample, wherein at least one probe of the plurality of probes comprises a first oligonucleotide that specifically binds a biomarker (e.g., one of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, CD44, POSTN, NES, TERT, UMOD, SGK1, GPR37L1, ISG15, RGS5, NAPB, BASP1, RUNDC3A, NEFM, RAB3A, GNG3, KIF1A, ATP1A3, CNTN1, CELF4, SYN2, TUBB4A, GRIN1, SPOCD1, DDK1, TNC, GBE1, SMIM3, CLIC1, MT1X, CYR61, HBA2, HBB, HBA1, MALAT1, RBM25, SLC25A37, NKTR, LUC7L3, PNISR, MEG3, IFI44L, FAM133B, PNN, PLEKHA4, PTMS, BDP1, MTRNR2L12, SREK1, ARGLU1, XAF1, MTRNR2L8, SRRM2, DKK1, HS2ST1, EGR1, TCIM, FOS, RPL17, TNC, IFITM3, TMSB4X, TMSB10, COX6C, CLIC1, TCEAL9, and RPL12, or a byproduct, degradation product, or fragment, or precursor thereof) (e.g., gDNA, mRNA, or a byproduct, degradation product, or fragment, or precursor thereof) in the tissue sample, wherein the first oligonucleotide is conjugated to a second oligonucleotide having a sequence, and wherein (i) each of the at least one probe comprises a first oligonucleotide that specifically binds a different biomarker of the tissue sample, and (ii) the first oligonucleotide of each of the at least one probe is conjugated to a different second oligonucleotide having a sequence; imaging the tissue sample to identify a location of interest of the tissue sample; and sequencing all or a portion of the sequence(s) of the second oligonucleotide(s) or a complement thereof, from the at least one probe specifically bound to the biomarker in the location of interest of the tissue sample, and using the determined sequence(s) to determine the presence or level of the biomarker in the sample (e.g., brain tissue or cerebrospinal fluid) or to determine the presence or level of the biomarker at the location in the sample (e.g., brain tissue).

(h) Methods of Detecting Biomarker(s) that Co-Localize with Another Biomarker in a Location in a Sample

In some instances, the methods can include detecting expression of a first biomarker in a biological sample and then detecting colocalized expression of various second biomarkers with the first biomarker. For example in some instances, a first biomarker can be a protein or nucleic acid (i.e., mRNA) biomarker that is specific to a cell of interest. Then, the methods include detecting dysregulated nucleic acid biomarker expression in the cell of interest.

In some instances, the first biomarker is an astrocyte biomarker. In some instances, the first biomarker is glial fibrillary acidic protein (GFAP). Detection of GFAP can be determined using any method known in the art, including fluorescent detection. In some instances, fluorescence detection is achieved using an antibody. In some instances, detection of the antibody is amplified using e.g., a secondary antibody. In some instances, the antibody is conjugated to a fluorophore (e.g., GFAP-Alexa 647, clone 644704).

In some instances, the first biomarker is a microglia biomarker. In some instances, the first biomarker is Ionized calcium-binding adaptor molecule 1 (IBA1). Detection of IBA1 can be determined using any method known in the art, including fluorescent detection. In some instances, fluorescence detection is achieved using an antibody. In some instances, detection of the antibody is amplified using e.g., a secondary antibody. In some instances, the antibody is conjugated to a fluorophore (e.g., IBA1, clone EPR16588).

In some instances, co-localized second biomarkers can be identified as expressed in the same spot on an array at a first biomarker, when the first biomarker is expressed at low abundances (i.e., less than 5%, less than 10%, less than 15%, less than 20%, less than 25%, or less than 30% expression) compared to the average expression of the average spot on a sample.

In some instances, co-localized second biomarkers can be identified as expressed in the same spot on an array at a first biomarker, when the first biomarker is expressed at high abundances (i.e., greater than 50%, 55%, 60%, 65%, 70%, 75%, 80%, 85%, 90%, 95%, 100% or more) compared to the average expression of the average spot on a sample.

In some instances, the increase of the first biomarker (e.g., IBA1) at a spot on an array can be determined by measuring the number of first biomarkers that associate with the capture probes at the spot. In some instances, an arbitrary number such as a log fold change increase (e.g., a 1.5-increase on a log 2 scale compared to the average number of biomarkers that associate with the capture probes at each spot on an array) can be used to identify regions of the sample with increased expression of the first biomarker. In some instances, the log fold change is at least greater than 1.5-fold, at least 1.6-fold, 1.7-fold, 1.8-fold, 1.9-fold, 2.0-fold or greater compared to the average number of biomarkers that associate with the capture probes at each spot on an array.

In some instances, the decrease of the first biomarker (e.g., IBA1) at a spot on an array can be determined by measuring the number of first biomarkers that associate with the capture probes at the spot. In some instances, an arbitrary number such as a log fold change (e.g., less than or equal to a 1.5-increase on a log 2 scale compared to the average number of biomarkers that associate with the capture probes at each spot on an array) can be used to identify regions of the sample with increased expression of the first biomarker. In some instances, the log fold change increase is about a 0.7-fold, 0.8-fold, 0.9-fold, 1.0-fold, 1.2-fold, 1.3-fold, 1.4-fold, 1 or 0.5-fold change compared to the average number of biomarkers that associate with the capture probes at each spot on an array.

Any of the exemplary methods described herein can be used to determine a level and/or at least one activity of one or more second biomarkers (i.e., a biomarker that co-localizes with a first biomarker. In some instances, the second biomarker includes one or more of HBA2, HBB, HBA1, COL1A2, MALAT1, RBM25, SLC25A37, NKTR, LUC7L3, ATP1A2, PNISR, MEG3, IFI44L, FAM133B, PNN, PLEKHA4, PTMS, BDP1, MTRNR2L12, SREK1, ARGLU1, XAF1, MTRNR2L8, SRRM2, COL4A1, DKK1, CHI3L1, HS2ST1, EGR1, TCIM, PLIN2, APOC1, FOS, MGP, SPP1, RPL17, TNC, IFITM3, MT2A, TMSB4X, TMSB10, PDPN, COX6C, VIM, CLIC1, IFITM2, TCEAL9, RPL12, TAGLN, and NAMPT, or a byproduct, degradation product, or fragment, or precursor thereof.

In some embodiments, the methods can include contacting the sample with a binding agent that specifically binds to one or more of the following second biomarkers: HBA2, HBB, HBA1, COL1A2, MALAT1, RBM25, SLC25A37, NKTR, LUC7L3, ATP1A2, PNISR, MEG3, IFI44L, FAM133B, PNN, PLEKHA4, PTMS, BDP1, MTRNR2L12, SREK1, ARGLU1, XAF1, MTRNR2L8, SRRM2, COL4A1, DKK1, CHI3L1, HS2ST1, EGR1, TCIM, PLIN2, APOC1, FOS, MGP, SPP1, RPL17, TNC, IFITM3, MT2A, TMSB4X, TMSB10, PDPN, COX6C, VIM, CLIC1, IFITM2, TCEAL9, RPL12, TAGLN, and NAMPT, or a byproduct, degradation product, or fragment, or precursor thereof) (e.g., gDNA, mRNA, a protein, or a byproduct, degradation product, or fragment, or precursor thereof), wherein the binding agent further comprises an oligonucleotide having a sequence; and sequencing all or a portion of the sequence of the oligonucleotide or a complement thereof, from a probe specifically bound to the second biomarker (e.g., one of HBA2, HBB, HBA1, COL1A2, MALAT, RBM25, SLC25A37, NKTR, LUC7L3, ATP1A2, PNISR, MEG3, IFI44L, FAM133B, PNN, PLEKHA4, PTMS, BDP1, MTRNR2L12, SREK1, ARGLU1, XAF1, MTRNR2L8, SRRM2, COL4A1, DKK1, CHI3L1, HS2ST1, EGR1, TCIM, PLIN2, APOC1, FOS, MGP, SPP1, RPL17, TNC, IFITM3, MT2A, TMSB4X, TMSB10, PDPN, COX6C, VIM, CLIC1, IFITM2, TCEAL9, RPL12, TAGLN, and NAMPT, or a byproduct, degradation product, or fragment, or precursor thereof), to determine the level of the second biomarker (e.g., HBA2, HBB, HBA1, COL1A2, MALAT1, RBM25, SLC25A37, NKTR, LUC7L3, ATP1A2, PNISR, MEG3, IFI44L, FAM133B, PNN, PLEKHA4, PTMS, BDP1, MTRNR2L12, SREK1, ARGLU1, XAF1, MTRNR2L8, SRRM2, COL4A1, DKK1, CHI3L1, HS2ST1, EGR1, TCIM, PLIN2, APOC1, FOS, MGP, SPP1, RPL17, TNC, IFITM3, MT2A, TMSB4X, TMSB10, PDPN, COX6C, VIM, CLIC1, IFITM2, TCEAL9, RPL12, TAGLN, and NAMPT, or a byproduct, degradation product, or fragment, or precursor thereof) in the sample (e.g., cerebrospinal fluid or brain tissue) or at a location in the sample (e.g., brain tissue).

In some embodiments, the methods can include delivering a plurality of probes to a sample (e.g., a tissue sample, for instance, affixed to a support), wherein a probe of the plurality of probes includes a protein that specifically binds to a second biomarker (e.g., one of HBA2, HBB, HBA1, COL1A2, MALAT1, RBM25, SLC25A37, NKTR, LUC7L3, ATP1A2, PNISR, MEG3, IFI44L, FAM133B, PNN, PLEKHA4, PTMS, BDP1, MTRNR2L12, SREK1, ARGLU1, XAF1, MTRNR2L8, SRRM2, COL4A1, DKK1, CHI3L1, HS2ST1, EGR1, TCIM, PLIN2, APOC1, FOS, MGP, SPP1, RPL17, TNC, IFITM3, MT2A, TMSB4X, TMSB10, PDPN, COX6C, VIM, CLIC1, IFITM2, TCEAL9, RPL12, TAGLN, and NAMPT, or a byproduct, degradation product, or fragment, or precursor thereof) (e.g., a protein, or a byproduct, degradation product, or fragment, or precursor thereof) in the tissue sample, wherein the protein is conjugated to an oligonucleotide having a sequence, and separating the probe specifically bound to the biomarker at the location of the tissue sample from the plurality of probes not specifically bound to the biomarker at the location of the tissue sample; and sequencing all or a portion of the sequence of the oligonucleotide or a complement thereof, from the specifically bound probe, and using the determined sequence to determine the level of the biomarker in a sample (e.g., cerebrospinal fluid or brain tissue) or to associate presence or abundance of the biomarker with the location of the tissue sample (e.g., brain tissue).

In some embodiments, the methods can include delivering a plurality of probes to a sample (e.g., a tissue sample, for instance, affixed to a support), wherein a probe of the plurality of probes includes a first oligonucleotide that specifically binds to a second biomarker (e.g., one of HBA2, HBB, HBA1, COL1A2, MALAT1, RBM25, SLC25A37, NKTR, LUC7L3, ATP1A2, PNISR, MEG3, IFI44L, FAM133B, PNN, PLEKHA4, PTMS, BDP1, MTRNR2L12, SREK1, ARGLU1, XAF1, MTRNR2L8, SRRM2, COL4A1, DKK1, CI3L1, HS2ST1, EGR1, TCIM, PLIN2, APOC1, FOS, MGP, SPP1, RPL17, TNC, IFITM3, MT2A, TMSB4X, TMSB10, PDPN, COX6C, VIM, CLIC1, IFITM2, TCEAL9, RPL12, TAGLN, and NAMPT, or a byproduct, degradation product, or fragment, or precursor thereof) (e.g., gDNA, mRNA, or a byproduct, degradation product, or fragment, or precursor thereof) in the tissue sample, wherein the first oligonucleotide is conjugated to a second oligonucleotide having a sequence, and separating the probe specifically bound to the biomarker at the location of the tissue sample from the plurality of probes not specifically bound to the biomarker at the location of the tissue sample; and sequencing all or a portion of the sequence of the second oligonucleotide or a complement thereof, from the specifically bound probe, and using the determined sequence to determine the presence or level of the biomarker in the sample (e.g., brain tissue or cerebrospinal fluid) or to determine the presence or level of the biomarker at the location in the sample (e.g., brain tissue).

In some embodiments, the methods can include delivering a plurality of probes to a tissue sample, wherein at least one probe of the plurality of probes comprises a protein that specifically binds to a second biomarker (e.g., one of HBA2, HBB, HBA1, COL1A2, MALAT1, RBM25, SLC25A37, NKTR, LUC7L3, ATP1A2, PNISR, MEG3, IFI44L, FAM133B, PNN, PLEKHA4, PTMS, BDP1, MTRNR2L12, SREK1, ARGLU1, XAF1, MTRNR2L8, SRRM2, COL4A1, DKK1, CHI3L1, HS2ST1, EGR1, TCIM, PLIN2, APOC1, FOS, MGP, SPP1, RPL17, TNC, IFITM3, MT2A, TMSB4X, TMSB10, PDPN, COX6C, VIM, CLIC1, IFITM2, TCEAL9, RPL12, TAGLN, and NAMPT, or a byproduct, degradation product, or fragment, or precursor thereof) (e.g., a protein, or a byproduct, degradation product, precursor, or fragment of any thereof) in the tissue sample, wherein the protein is conjugated to an oligonucleotide having a sequence, and wherein (i) each of the at least one probe comprises a protein that specifically binds a different biomarker of the tissue sample, and (ii) the protein of each of the at least one probe is conjugated to a different oligonucleotide having a sequence; imaging the tissue sample to identify a location of interest of the tissue sample; and sequencing all or a portion of the sequence(s) of the oligonucleotide(s) or a complement thereof, from the at least one probe specifically bound to the biomarker in the location of interest of the tissue sample, and using the determined sequence(s) to determine the presence or level of the biomarker in the sample (e.g., brain tissue or cerebrospinal fluid) or to determine the presence or level of the biomarker at the location in the sample (e.g., brain tissue).

In some embodiments, the methods can include delivering a plurality of probes to a tissue sample, wherein at least one probe of the plurality of probes comprises a first oligonucleotide that specifically binds a second biomarker (e.g., one of HBA2, HBB, HBA1, COL1A2, MALAT1, RBM25, SLC25A37, NKTR, LUC7L3, ATP1A2, PNISR, MEG3, IFI44L, FAM133B, PNN, PLEKHA4, PTMS, BDP1, MTRNR2L12, SREK1, ARGLU1, XAF1, MTRNR2L8, SRRM2, COL4A1, DKK1, CHI3L1, HS2ST1, EGR1, TCIM, PLIN2, APOC1, FOS, MGP, SPP1, RPL17, TNC, IFITM3, MT2A, TMSB4X, TMSB10, PDPN, COX6C, ViM, CLIC1, IFITM2, TCEAL9, RPL12, TAGLN, and NAMPT, or a byproduct, degradation product, or fragment, or precursor thereof) (e.g., gDNA, mRNA, or a byproduct, degradation product, or fragment, or precursor thereof) in the tissue sample, wherein the first oligonucleotide is conjugated to a second oligonucleotide having a sequence, and wherein (i) each of the at least one probe comprises a first oligonucleotide that specifically binds a different biomarker of the tissue sample, and (ii) the first oligonucleotide of each of the at least one probe is conjugated to a different second oligonucleotide having a sequence; imaging the tissue sample to identify a location of interest of the tissue sample; and sequencing all or a portion of the sequence(s) of the second oligonucleotide(s) or a complement thereof, from the at least one probe specifically bound to the biomarker in the location of interest of the tissue sample, and using the determined sequence(s) to determine the presence or level of the biomarker in the sample (e.g., brain tissue or cerebrospinal fluid) or to determine the presence or level of the biomarker at the location in the sample (e.g., brain tissue).

(i) Methods of (1) Diagnosing Glioblastoma and Identifying a Subject with an Increased Likelihood of Developing Glioblastoma; (2) Methods of Treating Glioblastoma; (3) Monitoring the Progression of Glioblastoma; and (4) Determining the Efficacy of a Treatment for Glioblastoma; in a Subject

(1) Methods of Diagnosing Glioblastoma and Identifying a Subject with an Increased Likelihood of Developing Glioblastoma

Provided herein are methods of diagnosing a subject as having glioblastoma. Also provided herein are methods of identifying a subject as having an increased likelihood of having glioblastoma. Further provided herein are methods of monitoring the progression of glioblastoma in a subject. Also provided herein are methods for determining the efficacy of a treatment for glioblastoma in a subject. Further provided herein are methods for treating glioblastoma in a subject.

In any of these methods, a biological sample can be any appropriate biological sample. In some embodiments, a biological sample can be a sample comprising brain tissue or cerebrospinal fluid. In some embodiments, the biological sample comprises blood, serum, plasma, or a cell culture sample. In some embodiments, the method can further include obtaining the sample from the subject. In some embodiments, the method can further include obtaining first and second biological samples from the subject.

In some embodiments, the methods of diagnosing a subject as having glioblastoma, or an increased likelihood of having glioblastoma can include (a) determining a level of one or more of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, CD44, POSTN, NES, TERT, UMOD, SGK1, GPR37L1, ISG15, RGS5, SPOCD1, DDK1, TNC, GBE1, SMIM3, CLIC1, MT1X, and CYR61, or a byproduct or precursor or degradation product or fragment thereof, in a biological sample from a subject; and (b) identifying a subject having an elevated level of one or more of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, CD44, POSTN, NES, TERT, UMOD, SGK1, GPR37L1, ISG15, RGS5, SPOCD1, DDK1, TNC, GBE1, SMIM3, CLIC1, MT1X, and CYR61 or a byproduct or precursor or degradation product or fragment thereof, as compared to a reference level, as having glioblastoma, or having an increased likelihood of having glioblastoma. In some embodiments, the method can include (a) determining a level of one or more of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, NAPB, BASP1, RUNDC3A, NEFM, RAB3A, GNG3, KIF1A, ATP1A3, CNTN1, CELF4, SYN2, TUBB4A, and GRIN1, or a byproduct or precursor or degradation product or fragment thereof, in a biological sample from a subject; and (b) identifying a subject having an decreased level of one or more of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, NAPB, BASP1, RUNDC3A, NEFM, RAB3A, GNG3, KIF1A, ATP1A3, CNTN1, CELF4, SYN2, TUBB4A, and GRIN1, or a byproduct or precursor or degradation product or fragment thereof, in the biological sample as compared to a reference level, as having glioblastoma, or having an increased likelihood of developing glioblastoma.

Also provided herein are methods of diagnosing a subject as having glioblastoma, or an increased likelihood of having glioblastoma. In some embodiments, the methods can include (a) determining the abundance (e.g., protein or mRNA) of IBA1; (b) determining a level of one or more of DKK1, CHI3L1, HS2ST1, EGR1, TCIM, PLIN2, APOC1, FOS, MGP, SPP1, RPL17, TNC, IFITM3, MT2A, TMSB4X, TMSB10, PDPN, COX6C, VIM, CLIC1, IFITM2, TCEAL9, RPL12, TAGLN, and NAMPT, or a byproduct or precursor or degradation product or fragment thereof, in a colocalized area of IBA1 expression in a biological sample from a subject; and (c) identifying a subject having an elevated level of one or more of DKK1, CHI3L1, HS2ST1, EGR1, TCIM, PLIN2, APOC1, FOS, MGP, SPP1, RPL17, TNC, IFITM3, MT2A, TMSB4X, TMSB10, PDPN, COX6C, VIM, CLIC1, IFITM2, TCEAL9, RPL12, TAGLN, and NAMPT or a byproduct or precursor or degradation product or fragment thereof, in the IBA1 co-localized area as compared to a reference level, as having glioblastoma, or having an increased likelihood of having glioblastoma.

In some embodiments, the methods can include (a) determining a level of one or more of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, CD44, POSTN, NES, TERT, UMOD, SGK1, GPR37L1, ISG15, RGS5, NAPB, BASP1, RUNDC3A, NEFM, RAB3A, GNG3, KIF1A, ATP1A3, CNTN1, CELF4, SYN2, TUBB4A, and GRIN1, or a byproduct or precursor or degradation product or fragment thereof, in a biological sample from a subject; and (b) identifying a subject having an decreased level of one or more of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, CD44, POSTN, NES, TERT, UMOD, SGK1, GPR37L1, ISG15, RGS5, NAPB, BASP1, RUNDC3A, NEFM, RAB3A, GNG3, KIF1A, ATP1A3, CNTN1, CELF4, SYN2, TUBB4A, and GRIN1, or a byproduct or precursor or degradation product or fragment thereof, in the biological sample as compared to a reference level, as having glioblastoma, or having an increased likelihood of having glioblastoma.

Also provided herein are methods of diagnosing a subject as having glioblastoma, or having an increased likelihood of having glioblastoma. In some embodiments, the methods can include (a) determining the abundance (e.g., protein or mRNA) of IBA1; (b) determining a level of one or more of HBA2, HBB, HBA1, COL1A2, MALAT1, RBM25, SLC25A37, NKTR, LUC7L3, ATP1A2, PNISR, MEG3, IFI44L, FAM133B, PNN, PLEKHA4, PTMS, BDP1, MTRNR2L12, SREK1, ARGLU1, XAF1, MTRNR2L8, SRRM2, and COL4A1, or a byproduct or precursor or degradation product or fragment thereof, in an IBA1 co-localized area in a biological sample from a subject; and (c) identifying a subject having an elevated level of one or more of HBA2, HBB, HBA1, COL1A2, MALAT1, RBM25, SLC25A37, NKTR, LUC7L3, ATP1A2, PNISR, MEG3, IFI44L, FAM133B, PNN, PLEKHA4, PTMS, BDP1, MTRNR2L12, SREK1, ARGLU1, XAF1, MTRNR2L8, SRRM2, and COL4A1, or a byproduct or precursor or degradation product or fragment thereof, in the IBA1 co-localized area as compared to a reference level, as having glioblastoma, or having an increased likelihood of having glioblastoma.

In some embodiments, the methods can further include confirming a diagnosis of glioblastoma in the subject. Non-limiting examples of ways to confirm a diagnosis of glioblastoma include obtaining an image of the subject's brain (e.g., a CT, MRI, or PET scan), detecting a genetic mutation associated with glioblastoma (e.g., a mutation associated with neurofibromatosis type 1, Turcot syndrome or Li Fraumeni syndrome), determining the levels of other biomarkers of glioblastoma, or performing neurological testing on the subject (e.g., vision, hearing, balance, coordination, strength and reflexes testing). Other methods of confirming a diagnosis of glioblastoma will be apparent to one skilled in the field.

In some embodiments, the methods can further comprise monitoring the identified subject for the development of symptoms of glioblastoma. In some embodiments, the methods can further include recording in the identified subject's clinical record that the subject has an increased likelihood of developing glioblastoma. In some embodiments, the methods can further include notifying the subject's family that the subject has an increased likelihood or susceptibility of developing glioblastoma.

In some embodiments, the methods can further include performing one or more tests to further determine the subject's risk of developing glioblastoma. Non-limiting examples of more tests to further determine the subject's risk of developing glioblastoma include, detecting a genetic mutation associated with glioblastoma (e.g., a mutation associated with neurofibromatosis type 1, Turcot syndrome, or Li Fraumeni syndrome), and determining the levels of other biomarkers (e.g., in brain tissue, cerebrospinal fluid, or in blood or a component thereof) indicative an increased risk of developing glioblastoma are indicative of an increased risk of developing glioblastoma.

In some embodiments, the methods can further include updating the subject's clinical record to indicate an increased risk of developing glioblastoma. In some embodiments, the methods can further include enrolling the subject in a clinical trial (e.g., for the early treatment and/or prevention of glioblastoma). In some embodiments, the methods can further include informing the subject's family of the subject's likelihood of developing glioblastoma. In some embodiments, the methods can further include monitoring the subject more frequently.

(2) Methods of Treating Glioblastoma

Provided herein are methods for treating a subject having glioblastoma with one or more therapeutic agents. In some embodiments, the methods can further include selecting a treatment for the subject. In some embodiments, the methods can further include administering a treatment of glioblastoma to the subject. In some embodiments, a treatment of glioblastoma can be a treatment that reduces the rate of progression of glioblastoma. In some embodiments, a treatment of glioblastoma can include surgery, radiation therapy, chemotherapy, targeted drug therapy, and tumor treating fields (TTF) therapy.

In some instances, the methods disclosed herein include treating a subject having glioblastoma with one or more therapeutic agents. Examples of therapeutic agents include, but are not limited to, e.g., chemotherapeutic agents, growth inhibitory agents, cytotoxic agents, agents used in radiation therapy, anti-angiogenesis agents, cancer immunotherapeutic agents, apoptotic agents, anti-tubulin agents, and other-agents (e.g., antibodies) to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib (Tarceva®), platelet derived growth factor inhibitors (e.g., Gleevec® (Imatinib Mesylate)), a COX-2 inhibitor (e.g., celecoxib), interferons, CTLA-4 inhibitors (e.g., anti-CTLA antibody ipilimumab (YERVOY®)), PD-1 inhibitors (e.g., anti-PD-1 antibodies, BMS-936558), PD-L1 inhibitors (e.g., anti-PD-L1 antibodies, MPDL3280A), PD-L2 inhibitors (e.g., anti-PD-L2 antibodies), TIM3 inhibitors (e.g., anti-TIM3 antibodies), cytokines, antagonists (e.g., neutralizing antibodies) that bind to one or more of the following targets ErbB2, ErbB3, ErbB4, PDGFR-beta, BlyS, APRIL, BCMA, PD-1, PD-L1, PD-L2, CTLA-4, TIM3, or VEGF receptor(s), TRAIL/Apo2, and other bioactive and organic chemical agents, etc. In some instances, the therapy or treatment includes surgery, chemotherapeutic agents, growth inhibitory agents, cytotoxic agents, agents used in radiation therapy, anti-angiogenesis agents, cancer immunotherapeutic agents, apoptotic agents, anti-tubulin agents, or a combination thereof. In some instances, chemotherapeutic agents are provided as a therapy to a subject having glioblastoma. Nonlimiting exemplary chemotherapeutic agents include anti-hormonal agents that act to regulate or inhibit hormone action on cancers such as anti-estrogens and selective estrogen receptor modulators (SERMs), including, for example, tamoxifen (including Nolvadex® tamoxifen), raloxifene, droloxifene, 4-hydroxytamoxifen, trioxifene, keoxifene, LY117018, onapristone, and Fareston® toremifene; aromatase inhibitors that inhibit the enzyme aromatase, which regulates estrogen production in the adrenal glands, such as, for example, 4(5)-imidazoles, aminoglutethimide, Megase® megestrol acetate, Aromasin® exemestane, formestanie, fadrozole, Rivisor® vorozole, Femara® letrozole, and Arimidex® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., Angiozyme® ribozyme) and a HER2 expression inhibitor; vaccines such as gene therapy vaccines, for example, Allovectin® vaccine, Leuvectin® vaccine, and Vaxid® vaccine; Proleukin® rIL-2; Lurtotecan® topoisomerase 1 inhibitor; Abarelix® rmRH; and pharmaceutically acceptable salts, acids or derivatives of any of the above.

In some embodiments, radiation therapy is administered locally to a tumor lesion to enhance the local immunogenicity of a subject's tumor (adjuvinating radiation) and/or to kill tumor cells (ablative radiation). In some instances, radiation therapy is administered systemically to a subject. In some instances, the radiation therapy is tomotherapy, stereotactic radiation, intensity-modulated radiation therapy (IMRT), hypofractionated radiotherapy, hypoxia-guided radiotherapy, and/or proton therapy. In some instances, radiation is followed by administration of a second therapy (e.g., chemotherapy, immunotherapy). In some instances, radiation is provided concurrently with administration of a second therapy (e.g., chemotherapy, immunotherapy).

In some instances, any of the above therapeutic agents are provided before, substantially contemporaneous with, or after other modes of treatment, for example, surgery, chemotherapy, radiation therapy, or the administration of a biologic, such as another therapeutic antibody. In some embodiments, the cancer has recurred or progressed following a therapy selected from surgery, chemotherapy, and radiation therapy, or a combination thereof.

In some instances, for treatment of cancer, as discussed herein, the antibodies are administered in conjunction with one or more additional anti-cancer agents, such as the chemotherapeutic agent, growth inhibitory agent, anti-angiogenesis agent and/or anti-neoplastic composition. Nonlimiting examples of chemotherapeutic agent, growth inhibitory agent, anti-angiogenesis agent, anti-cancer agent and anti-neoplastic composition.

In some embodiments, the methods can further include updating the subject's clinical record with the diagnosis of glioblastoma. In some embodiments, the methods can further include enrolling the subject in a clinical trial. In some embodiments, the methods can further include informing the subject's family of the diagnosis. In some embodiments, the methods can further include assessing or referring the subject for enrollment in a supportive care plan or care facility. In some embodiments, the methods can further include monitoring the subject more frequently.

(3) Methods of Monitoring the Progression of Glioblastoma in a Subject

In some embodiments, provided herein are methods of monitoring progression of glioblastoma in a subject over time. In some embodiments, the methods can include (a) determining a first level of one or more of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP, CD44, POSTN, NES, TERT, UMOD, SGK1, GPR37L1, ISG15, RGS5, SPOCD1, DDK1, TNC, GBE1, SMIM3, CLIC1, MT1X, and CYR61, or a byproduct or precursor or degradation product or fragment thereof, in a first biological sample obtained from a subject at a first time point; (b) determining a second level of one or more of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP, CD44, POSTN, NES, TERT, UMOD, SGK1, GPR37L1, ISG15, RGS5, SPOCD1, DDK1, TNC, GBE1, SMIM3, CLIC1, MT1X, and CYR61, or a byproduct or precursor or degradation product or fragment thereof, in a second biological sample obtained from the subject at a second time point; (c) identifying: (i) a subject having an increased second level as compared to the first level, as having progressing glioblastoma, or (ii) a subject having about the same or a decreased second level as compared to the first level, as having static or regressing glioblastoma.

In some embodiments, provided herein are methods of monitoring progression of glioblastoma in a subject over time. In some embodiments, the methods can include (a) determining an abundance of IBA1 (i.e., IBA1 protein or mRNA); (b) determining a first level of one or more of DKK1, CHI3L1, HS2ST1, EGR1, TCIM, PLIN2, APOC1, FOS, MGP, SPP1, RPL17, TNC, IFITM3, MT2A, TMSB4X, TMSB10, PDPN, COX6C, VIM, CLIC1, IFITM2, TCEAL9, RPL12, TAGLN, and NAMPT, or a byproduct or precursor or degradation product or fragment thereof, in an IBA1 co-localized area in a first biological sample obtained from a subject at a first time point; (c) determining a second level of one or more of DKK1, CHI3L1, HS2ST1, EGR1, TCIM, PLIN2, APOC1, FOS, MGP, SPP1, RPL17, TNC, IFITM3, MT2A, TMSB4X, TMSB10, PDPN, COX6C, VIM, CLIC1, IFITM2, TCEAL9, RPL12, TAGLN, and NAMPT, or a byproduct or precursor or degradation product or fragment thereof, in the IBA1 co-localized area in a second biological sample obtained from the subject at a second time point; (d) identifying: (i) a subject having an increased second level as compared to the first level, as having progressing glioblastoma, or (ii) a subject having about the same or a decreased second level as compared to the first level, as having static or regressing glioblastoma.

In some embodiments, the methods can include (a) determining a first level of one or more of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, NAPB, BASP1, RUNDC3A, NEFM, RAB3A, GNG3, KIF1A, ATP1A3, CNTN1, CELF4, SYN2, TUBB4A, and GRIN1, or a byproduct or precursor or degradation product or fragment thereof, in a first biological sample obtained from a subject at a first time point; (b) determining a second level of one or more of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, NAPB, BASP1, RUNDC3A, NEFM, RAB3A, GNG3, KIF1A, ATP1A3, CNTN1, CELF4, SYN2, TUBB4A, and GRIN1, or a byproduct or precursor or degradation product or fragment thereof, in a second biological sample obtained from the subject at a second time point; (c) identifying: (i) a subject having about the same or a decreased second level as compared to the first level of the one or more diagnostic biomarkers described herein, as having progressing glioblastoma, or (ii) a subject having an increased second level as compared to the first level, as having static or regressing glioblastoma.

In some embodiments, the methods can include (a) determining an abundance of IBA1 (i.e., IBA1 protein or mRNA); (b) determining a first level of one or more of HBA2, HBB, HBA1, COL1A2, MALAT1, RBM25, SLC25A37, NKTR, LUC7L3, ATP1A2, PNISR, MEG3, IFI44L, FAM133B, PNN, PLEKHA4, PTMS, BDP1, MTRNR2L12, SREK1, ARGLU1, XAF1, MTRNR2L8, SRRM2, and COL4A1, or a byproduct or precursor or degradation product or fragment thereof, in an IBA1 co-localized area in a first biological sample obtained from a subject at a first time point; (c) determining a second level of one or more of HBA2, HBB, HBA1, COL1A2, MALAT1, RBM25, SLC25A37, NKTR, LUC7L3, ATP1A2, PNISR, MEG3, IFI44L, FAM133B, PNN, PLEKHA4, PTMS, BDP1, MTRNR2L12, SREK1, ARGLU1, XAF1, MTRNR2L8, SRRM2, and COL4A1, or a byproduct or precursor or degradation product or fragment thereof, in the IBA1 co-localized area in a second biological sample obtained from the subject at a second time point; (d) identifying: (i) a subject having about the same or a decreased second level as compared to the first level of the one or more diagnostic biomarkers described herein, as having progressing glioblastoma, or (ii) a subject having an increased second level as compared to the first level, as having static or regressing glioblastoma.

In some embodiments, when the methods include identifying a subject as having progressing glioblastoma, the methods can further include administering a treatment for glioblastoma to the subject or increasing the dose of a previously administered treatment for glioblastoma to the subject. In some embodiments, the methods can further include selecting a treatment for glioblastoma for the subject. In some embodiments, the methods can further include administering a treatment of glioblastoma to the subject. In some embodiments, a treatment for glioblastoma can be a treatment that reduces the rate of progression of glioblastoma. In some embodiments, a treatment of glioblastoma can include surgery, radiation therapy, chemotherapy, targeted drug therapy, and tumor treating fields (TTF) therapy. In some embodiments, a treatment of glioblastoma can include palliative care. In some embodiments, the methods can further include updating the subject's clinical record that the subject has progressing glioblastoma. In some embodiments, the methods can further include enrolling the subject in a clinical trial. In some embodiments, the methods can further include informing the subject's family of the progression of the disease. In some embodiments, the methods can further include assessing or referring the subject for enrollment in a supportive care plan. In some embodiments, the methods can further include monitoring the subject more frequently.

In some embodiments, when the methods include identifying a subject as having static or regressing glioblastoma, the methods can include recording in the subject's clinical record that the subject has static or regressing glioblastoma. In some embodiments, the methods can further include the methods can further include maintaining the dose or lowering the dose of a treatment for glioblastoma to be administered to the subject or ceasing administration of a treatment for glioblastoma to the subject.

(4) Methods of Determining the Efficacy of a Treatment for Glioblastoma

In some embodiments, provided herein are methods of determining efficacy of treatment of a treatment for glioblastoma in a subject. In some embodiments, the method can include (a) determining a first level of one or more of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, CD44, POSTN, NES, TERT, UMOD, SGK1, GPR37L1, ISG15, RGS5, SPOCD1, DDK1, TNC, GBE1, SMIM3, CLIC1, MT1X, and CYR61, or a byproduct or precursor or degradation product or fragment thereof, in a first biological sample obtained from a subject at a first time point; (b) determining a second level of one or more of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, CD44, POSTN, NES, TERT, UMOD, SGK1, GPR37L1, ISG15, RGS5, SPOCD1, DDK1, TNC, GBE1, SMIM3, CLIC1, MT1X, and CYR61, or a byproduct or precursor or degradation product or fragment thereof, in a second biological sample obtained from the subject at a second time point, wherein the subject is administered one or more doses of a therapeutic treatment between the first and second time points; (c) identifying: (i) the therapeutic treatment as being effective in a subject having about the same or a decreased second level as compared to the first level, or (ii) the therapeutic treatment as not being effective in a subject having an increased second level as compared to the first level.

In some embodiments, provided herein are methods of determining efficacy of treatment of a treatment for glioblastoma in a subject. In some embodiments, the method can include (a) determining an abundance of IBA1 (i.e., IBA1 protein or mRNA); (b) determining a first level of one or more of DKK1, CHI3L1, HS2ST1, EGR1, TCIM, PLIN2, APOC1, FOS, MGP, SPP1, RPL17, TNC, IFITM3, MT2A, TMSB4X, TMSB10, PDPN, COX6C, VIM, CLIC1, IFITM2, TCEAL9, RPL12, TAGLN, and NAMPT, or a byproduct or precursor or degradation product or fragment thereof, in an IBA1 co-localized area in a first biological sample obtained from a subject at a first time point; (c) determining a second level of one or more of DKK1, CHI3L1, HS2ST1, EGR1, TCIM, PLIN2, APOC1, FOS, MGP, SPP1, RPL17, TNC, IFITM3, MT2A, TMSB4X, TMSB10, PDPN, COX6C, VIM, CLIC1, IFITM2, TCEAL9, RPL12, TAGLN, and NAMPT, or a byproduct or precursor or degradation product or fragment thereof, in the IBA1 co-localized area in a second biological sample obtained from the subject at a second time point, wherein the subject is administered one or more doses of a therapeutic treatment between the first and second time points; (d) identifying: (i) the therapeutic treatment as being effective in a subject having about the same or a decreased second level as compared to the first level, or (ii) the therapeutic treatment as not being effective in a subject having an increased second level as compared to the first level. In some embodiments, the method can include (a) determining a first level of one or more of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, NAPB, BASP1, RUNDC3A, NEFM, RAB3A, GNG3, KIF1A, ATP1A3, CNTN1, CELF4, SYN2, TUBB4A, and GRIN1, or a byproduct or precursor or degradation product or fragment thereof, in a first biological sample obtained from a subject at a first time point; (b) determining a second level of one or more of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, NAPB, BASP1, RUNDC3A, NEFM, RAB3A, GNG3, KIF1A, ATP1A3, CNTN1, CELF4, SYN2, TUBB4A, and GRIN1, or a byproduct or precursor or degradation product or fragment thereof, in a second biological sample obtained from the subject at a second time point, wherein the subject is administered one or more doses of a therapeutic treatment between the first and second time points; (c) identifying: (i) the therapeutic treatment as being effective in a subject having an increased second level as compared to the first level, or (ii) the therapeutic treatment as not being effective in a subject having about the same or a decreased second level as compared to the first level.

In some embodiments, the method can include (a) determining an abundance of IBA1 (i.e., IBA1 protein or mRNA); (b) determining a first level of one or more of HBA2, HBB, HBA1, COL1A2, MALAT1, RBM25, SLC25A37, NKTR, LUC7L3, ATP1A2, PNISR, MEG3, IFI44L, FAM133B, PNN, PLEKHA4, PTMS, BDP1, MTRNR2L12, SREK1, ARGLU1, XAF1, MTRNR2L8, SRRM2, and COL4A1, or a byproduct or precursor or degradation product or fragment thereof, in an IBA1 co-localized area in a first biological sample obtained from a subject at a first time point; (c) determining a second level of one or more of HBA2, HBB, HBA1, COL1A2, MALAT1, RBM25, SLC25A37, NKTR, LUC7L3, ATP1A2, PNISR, MEG3, IFI44L, FAM133B, PNN, PLEKHA4, PTMS, BDP1, MTRNR2L12, SREK1, ARGLU1, XAF1, MTRNR2L8, SRRM2, and COL4A1, or a byproduct or precursor or degradation product or fragment thereof, in the IBA1 co-localized area in a second biological sample obtained from the subject at a second time point, wherein the subject is administered one or more doses of a therapeutic treatment between the first and second time points; (d) identifying: (i) the therapeutic treatment as being effective in a subject having an increased second level as compared to the first level, or (ii) the therapeutic treatment as not being effective in a subject having about the same or a decreased second level as compared to the first level. In some embodiments, the methods include identifying the therapeutic treatment as being effective in the subject. In some embodiments, the methods can further include selecting additional doses of the therapeutic treatment for the subject. In some embodiments, the methods can further include administering additional doses of the therapeutic treatment to the subject. In some embodiments, the methods can further include recording in the subject's clinical record that the therapeutic treatment is effective in the subject.

In some embodiments, the methods include identifying the therapeutic treatment as not being effective in the subject. In some embodiments, the methods can further include selecting a different therapeutic treatment for the subject. In some embodiments, the methods can further include administering a different therapeutic treatment to the subject. In some embodiments, the methods can further include increasing the dose of the therapeutic treatment to be administered to the subject. In some embodiments, the methods can include administering one or more additional doses of the therapeutic treatment to the subject in combination with an additional therapeutic treatment. In some embodiments, the methods can further include ceasing administration of the therapeutic treatment to the subject. In some embodiments, the methods can further include recording in the subject's clinical record that the therapeutic treatment is not effective in the subject. In some embodiments, the methods can further include referring the patient for enrollment in a clinical trial of a different therapeutic agent.

In some embodiments, the methods can further include additional assessments of the efficacy of the therapeutic treatment. Non-limiting examples of ways to assess efficacy of the therapeutic treatment include obtaining an image of the subject's brain (e.g., a CT, MRI, or PET scan), testing of other biomarkers, and performing neurological testing on the subject (e.g., vision, hearing, balance, coordination, strength and reflexes testing).

(m) Determining the Sequence of the One or More Analytes

(1) Pre-Capture Methods

(i) Imaging and Staining

Prior to addition of the probes, in some instances, biological samples can be stained using a wide variety of stains and staining techniques. In some embodiments, a sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the methods disclosed herein include imaging the biological sample. In some instances, imaging the sample occurs prior to deaminating the biological sample. In some instances, the sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation. In some instances, the stain is an H&E stain.

In some embodiments, the biological sample can be stained using a detectable label (e.g., radioisotopes, fluorophores, chemiluminescent compounds, bioluminescent compounds, and dyes) as described elsewhere herein.

In some embodiments, biological samples can be destained. Methods of destaining or discoloring a biological sample are known in the art, and generally depend on the nature of the stain(s) applied to the sample. For example, H&E staining can be destained by washing the sample in HCl, or any other acid.

In some instances, a biological sample can be imaged using a variety of different techniques, e.g., expansion microscopy, bright field microscopy, dark field microscopy, phase contrast microscopy, electron microscopy, fluorescence microscopy, reflection microscopy, interference microscopy, confocal microscopy, and visual identification (e.g., by eye), and combinations thereof.

Methods of staining and imaging are further disclosed in priority documents U.S. Provisional Patent Application Nos. 62/964,063, and 63/108,273, each of which is incorporated herein by reference in its entirety.

(ii) Preparation of Sample for Application to Array

In some instances, the biological sample is deparaffinized. Deparaffinization can be achieved using any method known in the art. For example, in some instances, the biological samples is treated with a series of washes that include xylene and various concentrations of ethanol. In some instances, the biological sample is decrosslinked. In some instances, the biological sample is decrosslinked in a solution containing TE buffer (comprising Tris and EDTA).

In some instances, the methods of preparing a biological sample for probe application include permeabilizing the sample. In some instances, the biological sample is permeabilized using a phosphate buffer. In some instances, the phosphate buffer is PBS (e.g., lx PBS). In some instances, the phosphate buffer is PBST (e.g., lx PBST). In some instances, the permeabilization step is performed multiple times (e.g., 3 times at 5 minutes each).

In some instances, the methods of preparing a biological sample for probe application include steps of equilibrating and blocking the biological sample. In some instances, equilibrating is performed using a pre-hybridization (pre-Hyb) buffer.

Methods of sample preparation are further disclosed in priority documents U.S. Provisional Patent Application Nos. 62/964,063, and 63/108,273, each of which is incorporated herein by reference in its entirety.

(2) Post Capture Methods

After the one or more analytes from the sample has hybridized or otherwise been associated with a capture probe according to any of the methods described above in connection with the general spatial cell-based analytical methodology, the barcoded constructs that result from hybridization/association are analyzed.

In some embodiments, after contacting a biological sample with a substrate that includes capture probes, a removal step can optionally be performed to remove all or a portion of the biological sample from the substrate. In some embodiments, the removal step includes enzymatic and/or chemical degradation of cells of the biological sample. For example, the removal step can include treating the biological sample with an enzyme (e.g., a proteinase, e.g., proteinase K) to remove at least a portion of the biological sample from the substrate. In some embodiments, the removal step can include ablation of the tissue (e.g., laser ablation).

In some embodiments, a biological sample is not removed from the substrate. For example, the biological sample is not removed from the substrate prior to releasing a capture probe (e.g., a capture probe bound to an analyte) from the substrate. In some embodiments, such releasing comprises cleavage of the capture probe from the substrate (e.g., via a cleavage domain). In some embodiments, such releasing does not comprise releasing the capture probe from the substrate (e.g., a copy of the capture probe bound to an analyte can be made and the copy can be released from the substrate, e.g., via denaturation). In some embodiments, the biological sample is not removed from the substrate prior to analysis of an analyte bound to a capture probe after it is released from the substrate. In some embodiments, the biological sample remains on the substrate during removal of a capture probe from the substrate and/or analysis of an analyte bound to the capture probe after it is released from the substrate. In some embodiments, the biological sample remains on the substrate during removal (e.g., via denaturation) of a copy of the capture probe (e.g., complement). In some embodiments, analysis of an analyte bound to capture probe from the substrate can be performed without subjecting the biological sample to enzymatic and/or chemical degradation of the cells (e.g., permeabilized cells) or ablation of the tissue (e.g., laser ablation).

In some embodiments, a capture probe can be extended (an “extended capture probe,” e.g., as described herein). For example, extending a capture probe can include generating cDNA from a captured (hybridized) RNA. This process involves synthesis of a complementary strand of the hybridized nucleic acid, e.g., generating cDNA based on the captured RNA template (the RNA hybridized to the capture domain of the capture probe). Thus, in an initial step of extending a capture probe, e.g., the cDNA generation, the captured (hybridized) nucleic acid, e.g., RNA, acts as a template for the extension, e.g., reverse transcription, step. In some embodiments, the capture probe is extended using reverse transcription. In some embodiments, a capture domain of a capture probe includes a primer for producing the complementary strand of a nucleic acid hybridized to the capture probe, e.g., a primer for DNA polymerase and/or reverse transcription.

In some embodiments, extended capture probes are amplified to yield quantities that are sufficient for analysis, e.g., via DNA sequencing. In some embodiments, the first strand of the extended capture probes (e.g., DNA and/or cDNA molecules) acts as a template for the amplification reaction (e.g., a polymerase chain reaction). In some embodiments, where the extended capture probe includes a cleavage domain, the extended capture probe is released from the surface of the substrate by cleavage. For example, the cleavage domain of the extended capture probe can be cleaved by any of the methods described herein. In some embodiments, the extended capture probe is released from the surface of the substrate, e.g., via cleavage of a cleavage domain in the extended capture probe, prior to the step of amplifying the extended capture probe.

In some instances, the one or more analytes and capture probe can be amplified or copied, creating a plurality of cDNA molecules. In some embodiments, cDNA can be denatured from the capture probe template and transferred (e.g., to a clean tube) for amplification, and/or library construction. The spatially-barcoded cDNA can be amplified via PCR prior to library construction. The cDNA can then be enzymatically fragmented and size-selected in order to optimize for cDNA amplicon size. P5 and P7 sequences directed to capturing the amplicons on a sequencing flowcell (Illumina sequencing instruments) can be appended to the amplicons, i7, and i5 can be used as sample indexes, and TruSeq Read 2 can be added via End Repair, A-tailing, Adaptor Ligation, and PCR. The cDNA fragments can then be sequenced using paired-end sequencing using TruSeq Read 1 and TruSeq Read 2 as sequencing primer sites. The additional sequences are directed toward Illumina sequencing instruments or sequencing instruments that utilize those sequences; however a skilled artisan will understand that additional or alternative sequences used by other sequencing instruments or technologies are also equally applicable for use in the aforementioned methods.

In some embodiments, where a sample is barcoded directly via hybridization with capture probes or analyte capture agents hybridized, bound, or associated with either the cell surface, or introduced into the cell, as described above, sequencing can be performed on the intact sample.

A wide variety of different sequencing methods can be used to analyze barcoded analyte (e.g., the one or more analytes). In general, sequenced polynucleotides can be, for example, nucleic acid molecules such as deoxyribonucleic acid (DNA) or ribonucleic acid (RNA), including variants or derivatives thereof (e.g., single stranded DNA or DNA/RNA hybrids, and nucleic acid molecules with a nucleotide analog).

Sequencing of polynucleotides can be performed by various systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based single plex methods, emulsion PCR), and/or isothermal amplification. Non-limiting examples of methods for sequencing genetic material include, but are not limited to, DNA hybridization methods (e.g., Southern blotting), restriction enzyme digestion methods, Sanger sequencing methods, next-generation sequencing methods (e.g., single-molecule real-time sequencing, nanopore sequencing, and Polony sequencing), ligation methods, and microarray methods.

Methods of post-capture detection are further disclosed in priority documents U.S. Provisional Patent Application Nos. 62/964,063, and 63/108,273, each of which is incorporated herein by reference in its entirety.

(n) Kits

In some embodiments, also provided herein are kits that include one or more reagents to detect a level of one or more of any of the biomarkers and/or candidate biomarkers described herein (e.g., COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, CD44, POSTN, NES, TERT, UMOD, SGK1, GPR37L1, ISG15, RGS5, NAPB, BASP1, RUNDC3A, NEFM, RAB3A, GNG3, KIF1A, ATP1A3, CNTN1, CELF4, SYN2, TUBB4A, GRIN1, SPOCD1, DDK1, TNC, GBE1, SMIM3, CLIC1, MT1X, CYR61, HBA2, HBB, HBA1, MALAT1, RBM25, SLC25A37, NKTR, LUC7L3, PNISR, MEG3, IFI44L, FAM133B, PNN, PLEKHA4, PTMS, BDP1, MTRNR2L12, SREK1, ARGLU1, XAF1, MTRNR2L8, SRRM2, DKK1, HS2ST1, EGR1, TCIM, FOS, RPL17, TNC, IFITM3, TMSB4X, TMSB10, COX6C, CLIC1, TCEAL9, and RPL12, or a byproduct or precursor or degradation product or fragment thereof).

In some embodiments, reagents can include one or more antibodies (and/or antigen-binding antibody fragments), labeled hybridization probes, and primers. For example, in some embodiments, an antibody (and/or antigen-binding antibody fragment) can be used for visualizing one or more features of a tissue sample (e.g., by using immunofluorescence or immunohistochemistry). In some embodiments, an antibody (and/or antigen-binding antibody fragment) can be an analyte binding moiety, for example, as part of an analyte capture agent. Useful commercially available antibodies will be apparent to one skilled in the art.

In some embodiments, labeled hybridization probes can be used for in situ sequencing of one or more biomarkers and/or candidate biomarkers. In some embodiments, primers can be used for amplification (e.g., clonal amplification) of a captured oligonucleotide analyte.

In some embodiments, a kit can further include instructions for performing any of the methods or steps provided herein.

### EXAMPLES

Identifying individual cells and their genetic makeup can be important for understanding how a system physiologically functions, develops, and organizes; as well as how these modalities are altered in diseased states. The Example described below demonstrates, e.g., the ability to do one or more of the following: (1) examine histological and transcriptome profiles from the same tissue section at a much higher resolution, better sensitivity, and shorter time; (2) obtain unbiased and high-throughput gene expression analysis for intact tissue sections across different brain regions; (3) generate spatial clustering that reliably correlates with anatomy; and or (4) demonstrate the ability to discover novel targets and/or pathways with unbiased analysis.

### Example 1—Human Brain Analyses

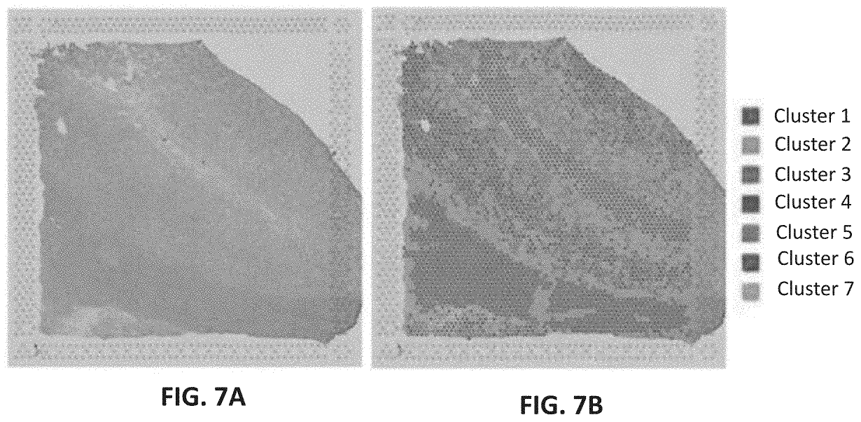

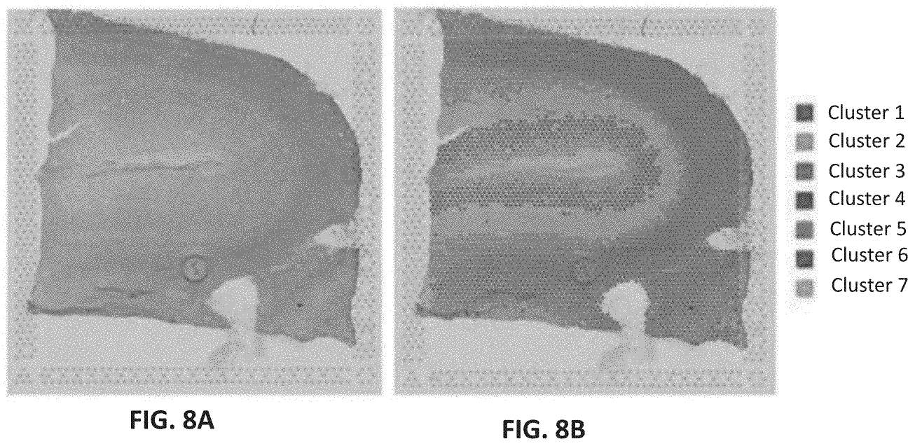

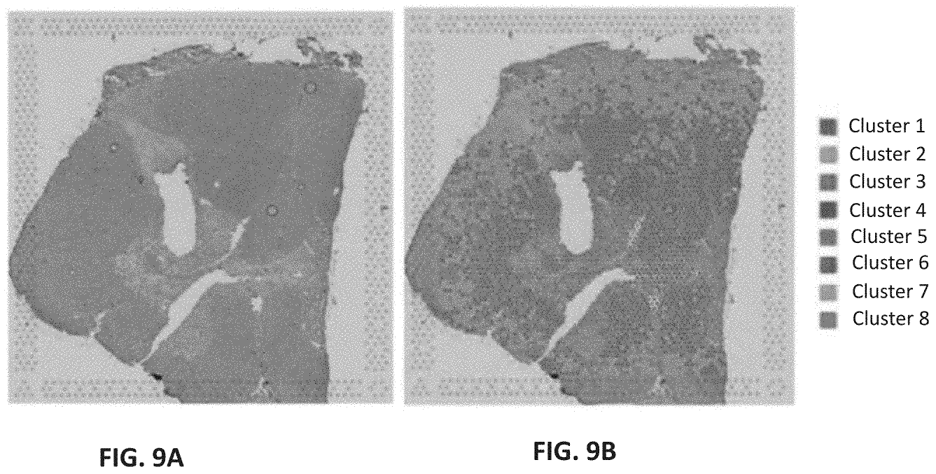

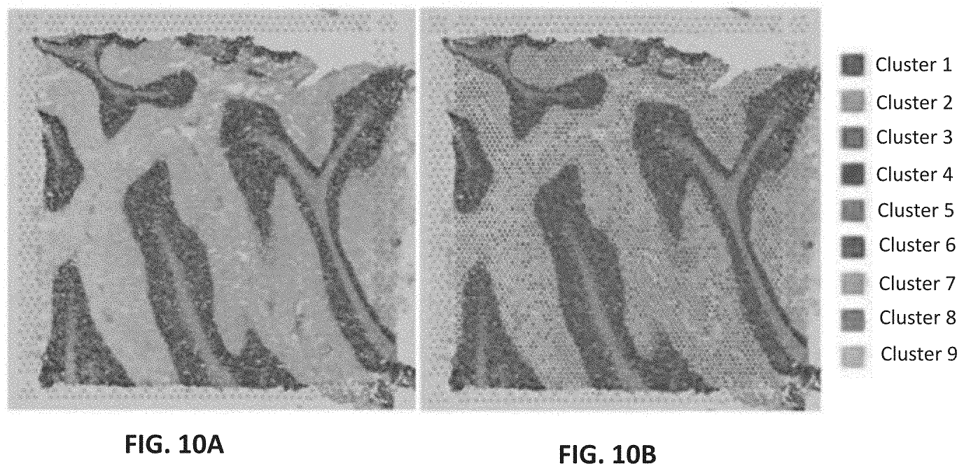

Spatial analysis was performed on an unspecified human cerebral cortex sample cord (FIGS. 7A and 7B), a temporal human cerebral cortex sample (FIGS. 8A and 8B), and human neuronal samples from spinal cord (FIGS. 9A and 9B) and cerebellum (FIGS. 10A and 10B).

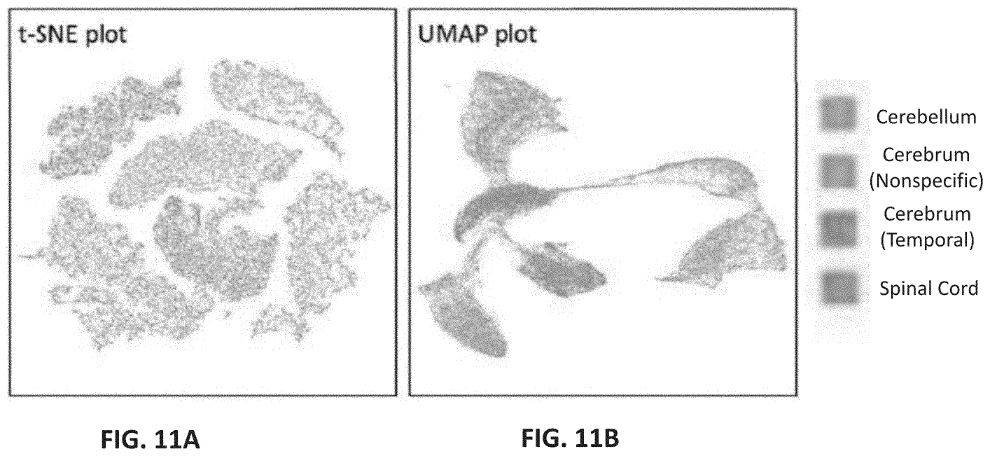

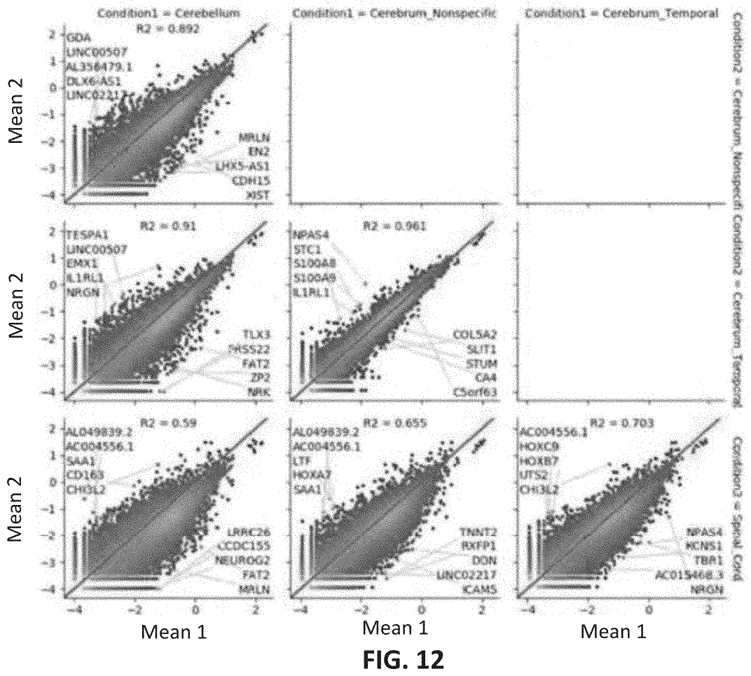

Multiple neuronal samples, i.e., cerebellum, cerebrum (non-specific), cerebrum (temporal), and spinal cord, were compared using t-SNE and UMAP plots (see FIGS. 11A and 11B). The t-SNE plot and UMAP plots demonstrated distinct cell type clustering and relationships, respectively. A scatter plot demonstrated the differential expression of genes captured using the methods described herein (FIG. 12). Comparison of different brain regions exhibited larger spread, compared to similar regions (e.g., cerebral tissues).

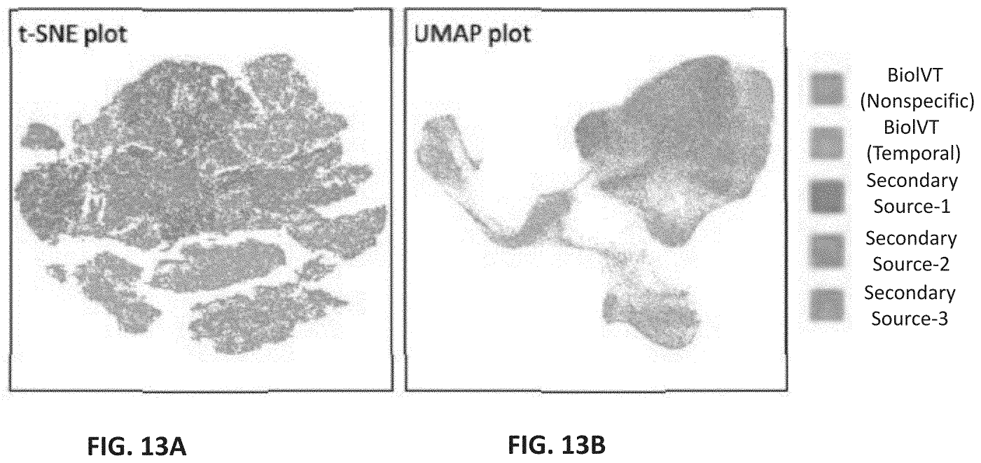

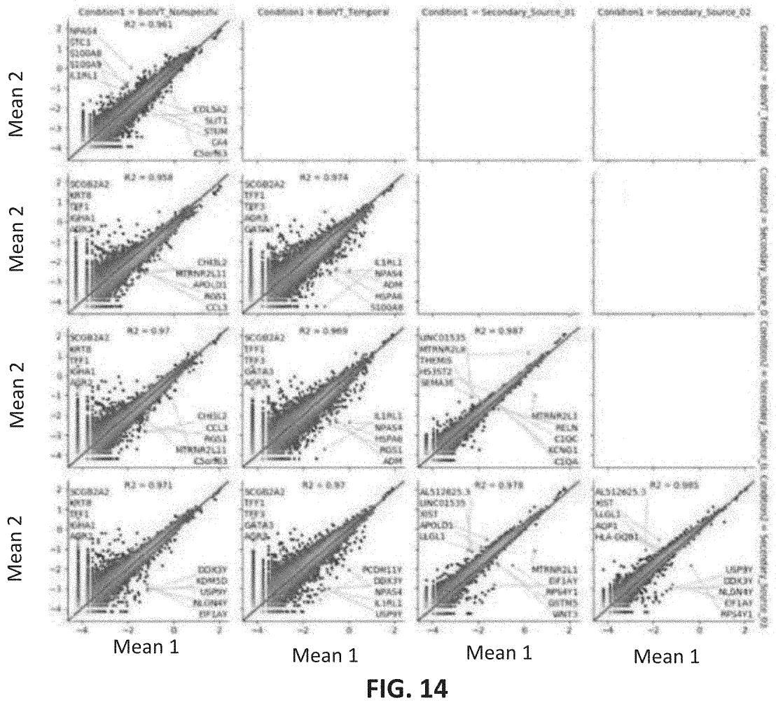

Cerebral tissues from different region/sources (BioIVT nonspecific, BioIVT temporal, secondary source 1, secondary source 2, and secondary source 3) were compared (FIGS. 13A and 13B). A t-SNE plot and UMAP plot demonstrated distinct cell type clustering and relationships, respectively. A scatter plot demonstrated the differential expression of genes captured using the methods described herein (FIG. 14).

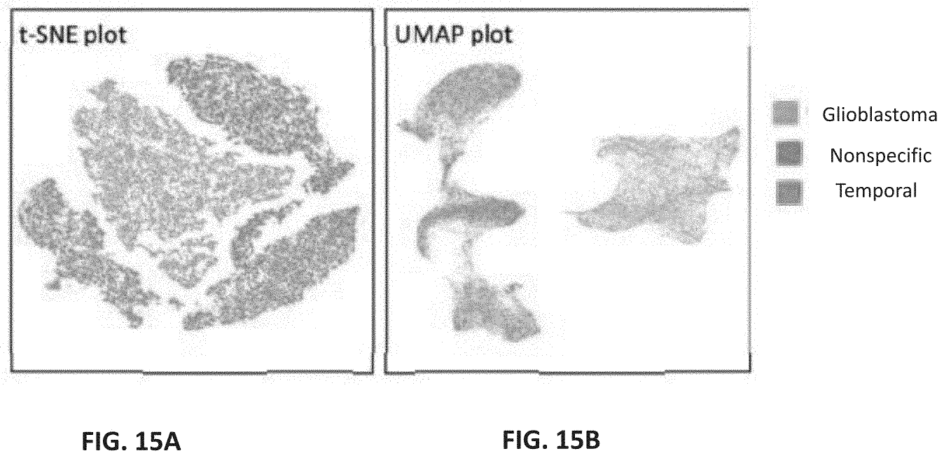

Healthy and glioblastoma samples from the cerebral cortex were also compared. A t-SNE plot and UMAP plot demonstrated distinct cell type clustering and relationships, respectively (FIGS. 15A and 15B).

### Example 2—Comparison Between Healthy and Glioblastoma Samples

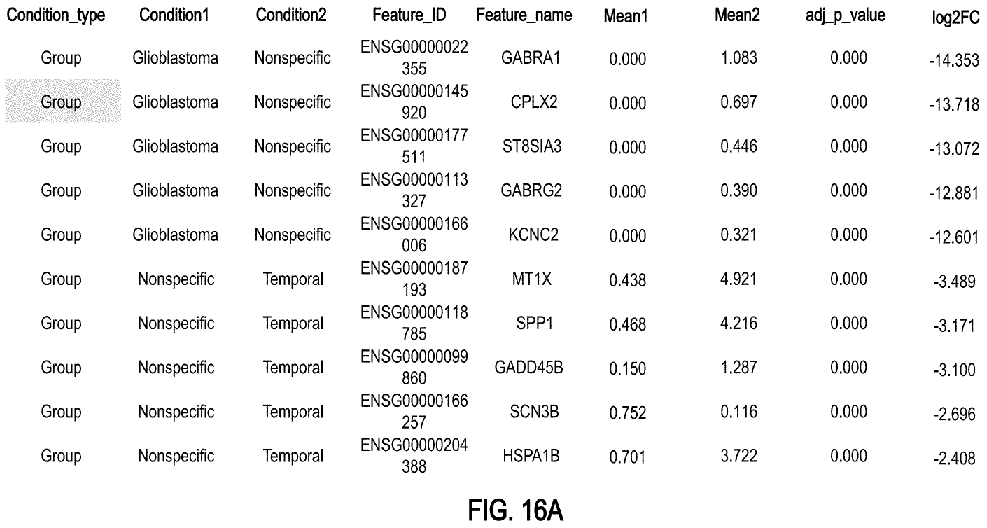

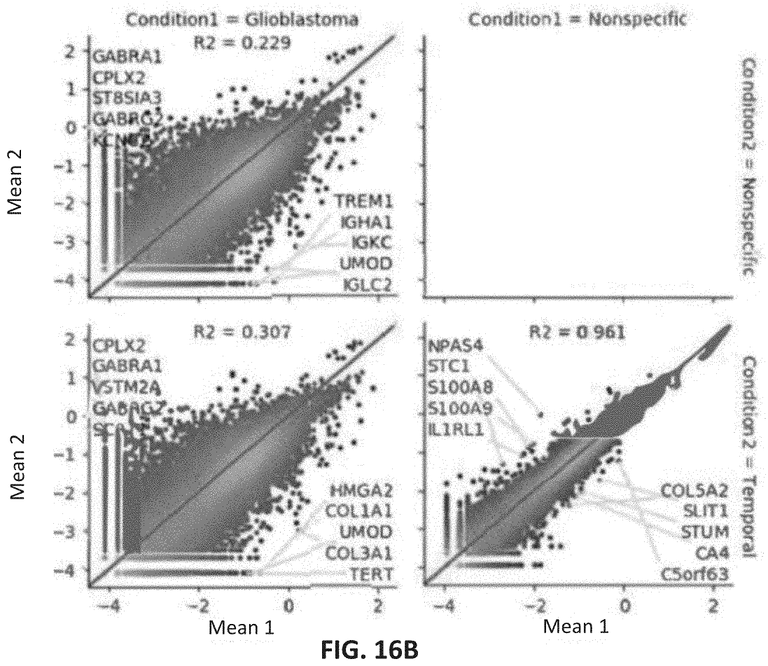

Spatial analysis was performed on healthy (nonspecific & temporal) and glioblastoma samples from different patients. A scatter plot demonstrated the differential expression of genes captured using the methods described herein (FIG. 16B). Decreased expression of GABRA1, CPLX2, ST8SIA3, GABRG2, and KCNC2 was observed in glioblastoma compared to healthy sample (FIG. 16A).

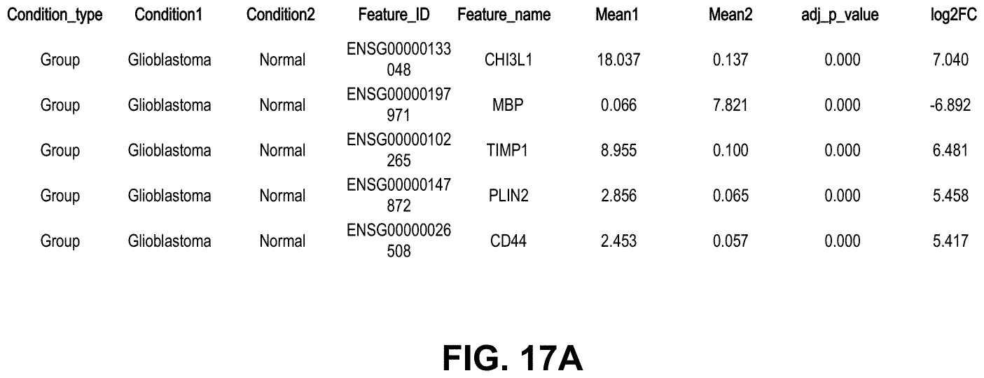

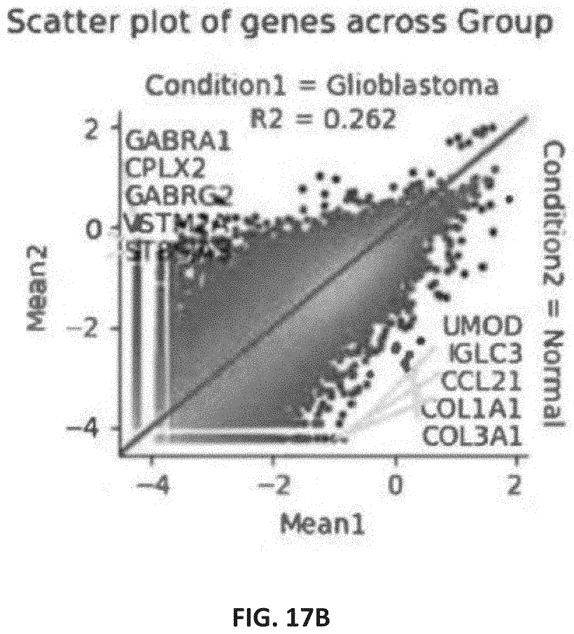

Pooled normal healthy and glioblastoma samples from different patients were compared. A scatter plot demonstrated the differential expression of genes captured using the methods described herein (FIG. 17B). Differentially expressed genes were observed. For example, overexpression of CHI3L1, TIMP1, PLIN2, and CD44 and underexpression of MBP within the glioblastoma sample were observed (FIG. 17A).

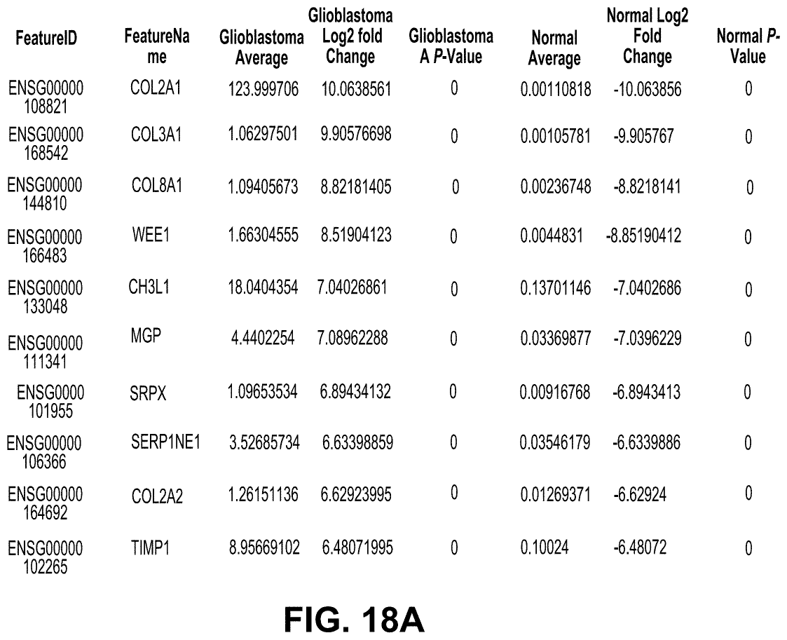

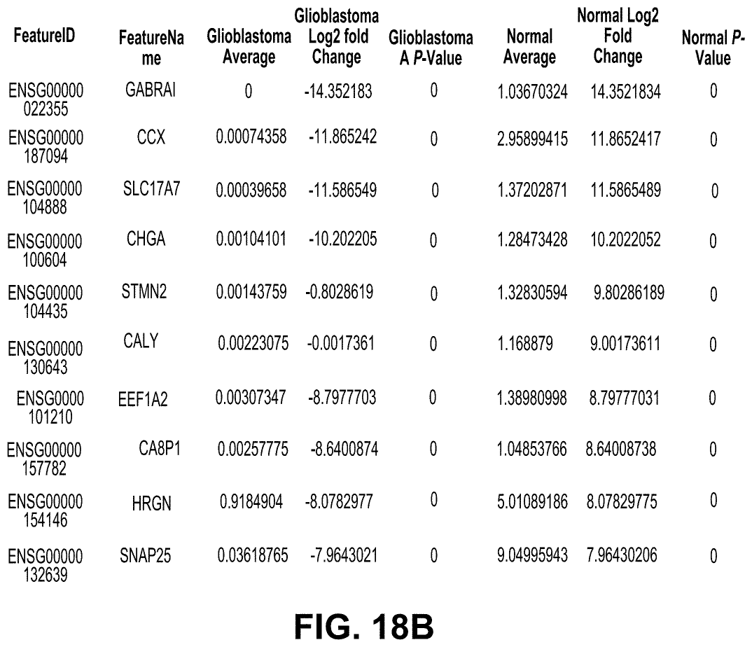

Comparison of between healthy and glioblastoma samples also showed other genes were differentially expressed, for example, the genes shown in FIGS. 18A and 18B as well as Tables 2 and 3.

TABLE 2
Top overexpressed genes in glioblastoma relative to normal

| | | Glioblastoma | | | Normal | |
|---|---|---|---|---|---|---|
| Feature | Glioblastoma | Log2 Fold | Glioblastoma | Normal | Log2 Fold | Normal |
| Name | Average | Change | P-Value | Average | Change | P-Value |
| COL1A1 | 1.23999706 | 10.0638561 | 0 | 0.00110818 | −10.063856 | 0 |
| COL3A1 | 1.06297501 | 9.90576698 | 0 | 0.00105781 | −9.905767 | 0 |
| COL8A1 | 1.09405673 | 8.82181405 | 0 | 0.00236748 | −8.8218141 | 0 |
| WEE1 | 1.66304555 | 8.51904123 | 0 | 0.0044831 | −8.5190412 | 0 |
| CHI3L1 | 18.0404354 | 7.04026861 | 0 | 0.13701146 | −7.0402686 | 0 |
| MGP | 4.4401254 | 7.03962288 | 0 | 0.03369877 | −7.0396229 | 0 |
| SRPX | 1.09653534 | 6.89434132 | 0 | 0.00916768 | −6.8943413 | 0 |
| SERPINE1 | 3.52695734 | 6.63398859 | 0 | 0.03546179 | −6.6339886 | 0 |
| COL1A2 | 1.26151136 | 6.62923995 | 0 | 0.01269371 | −6.62924 | 0 |
| TIMP1 | 8.95669102 | 6.48071995 | 0 | 0.10024 | −6.48072 | 0 |
| ANXA1 | 3.15377841 | 6.32864809 | 0 | 0.03918931 | −6.3286481 | 0 |
| COL6A2 | 1.06297501 | 5.85344594 | 0 | 0.01833536 | −5.8534459 | 0 |
| CAV1 | 1.20608973 | 5.56157824 | 0 | 0.02548816 | −5.5615782 | 0 |
| PLIN2 | 2.85624653 | 5.45801383 | 0 | 0.06492933 | −5.4580138 | 0 |
| CD44 | 2.4537209 | 5.41720583 | 0 | 0.05737355 | −5.4172058 | 0 |
| APOC1 | 5.78253847 | 5.3730687 | 0 | 0.13947968 | −5.3730687 | 0 |
| IGFBP2 | 4.66914861 | 5.36192666 | 0 | 0.1134878 | −5.3619267 | 0 |
| PDPN | 1.03333089 | 5.35572835 | 0 | 0.02518593 | −5.3557283 | 0 |
| VIM | 25.9392573 | 5.23652741 | 0 | 0.68797886 | −5.2365274 | 0 |
| LGALS3 | 6.94574832 | 4.90982887 | 0 | 0.23100535 | −4.9098289 | 0 |
| VEGFA | 5.15530241 | 4.75663587 | 0 | 0.19065749 | −4.7566359 | 0 |
| IGFBP5 | 2.99698179 | 4.62036276 | 0 | 0.12179916 | −4.6203628 | 0 |
| CTGF | 1.18611217 | 4.55321848 | 0 | 0.0504726 | −4.5532185 | 0 |
| EMP1 | 1.07288944 | 4.35483735 | 0 | 0.05238673 | −4.3548374 | 0 |
| EMP3 | 1.07353388 | 4.35155197 | 0 | 0.05253785 | −4.351552 | 0 |
| IGFBP3 | 1.07958168 | 4.28689993 | 0 | 0.05525793 | −4.2868999 | 0 |
| A2M | 4.80264633 | 4.26503459 | 0 | 0.24974368 | −4.2650346 | 0 |
| ANXA2 | 1.02544892 | 4.21795198 | 0 | 0.05505644 | −4.217952 | 0 |
| FLNA | 1.36933073 | 4.14183216 | 3.01E−306 | 0.07752229 | −4.1418322 | 3.01E−306 |
| IGFBP7 | 7.97159381 | 4.12851759 | 0 | 0.45571421 | −4.1285176 | 0 |
| S100A11 | 1.60574018 | 4.0907128 | 1.96E−302 | 0.09419538 | −4.0907128 | 1.96E−302 |
| ADM | 2.30331908 | 4.0587042 | 1.38E−299 | 0.13817001 | −4.0587042 | 1.38E−299 |
| FN1 | 1.64138253 | 4.03850975 | 6.14E−292 | 0.09983703 | −4.0385097 | 6.14E−292 |
| SERPING1 | 2.00553934 | 3.98757791 | 1.69E−291 | 0.126383 | −3.9875779 | 1.69E−291 |
| MT2A | 47.8913793 | 3.97755744 | 2.62E−295 | 3.04009285 | −3.9775574 | 2.62E−295 |
| S100A10 | 2.19525186 | 3.78345289 | 3.35E−266 | 0.15937656 | −3.7834529 | 3.35E−266 |
| SPARC | 5.0779699 | 3.77129812 | 8.99E−269 | 0.37184507 | −3.7712981 | 8.99E−269 |
| ITGB1 | 1.24768074 | 3.72288566 | 3.20E−257 | 0.09444724 | −3.7228857 | 3.20E−257 |
| SLC5A3 | 1.06183486 | 3.7215987 | 2.00E−255 | 0.08044386 | −3.7215987 | 2.00E−255 |
| FABP7 | 1.92766154 | 3.62028615 | 1.98E−247 | 0.15670686 | −3.6202862 | 1.98E−247 |
| YBX3 | 1.44121031 | 3.58458058 | 3.83E−242 | 0.12008651 | −3.5845806 | 3.83E−242 |
| IFITM2 | 1.64420815 | 3.57344722 | 8.11E−243 | 0.13806927 | −3.5734472 | 8.11E−243 |
| TAGLN2 | 1.1312854 | 3.55871738 | 6.46E−238 | 0.09595839 | −3.5587174 | 6.46E−238 |
| COL6A1 | 1.06917153 | 3.55021661 | 4.43E−238 | 0.09122344 | −3.5502166 | 4.43E−238 |
| HLA-A | 5.10359869 | 3.51334628 | 4.82E−240 | 0.44689914 | −3.5133463 | 4.82E−240 |
| LGALS3BP | 1.08384488 | 3.31576455 | 1.13E−211 | 0.10880322 | −3.3157645 | 1.13E−211 |
| ANXA5 | 1.56201757 | 3.29340226 | 4.49E−211 | 0.15927582 | −3.2934023 | 4.49E−211 |
| APOE | 23.4428053 | 3.2832261 | 4.26E−215 | 2.40797638 | −3.2832261 | 4.26E−215 |
| GADD45A | 1.28461197 | 3.24716836 | 8.61E−204 | 0.13524844 | −3.2471684 | 8.61E−204 |
| TPM4 | 1.83877873 | 3.15405038 | 5.04E−197 | 0.20652462 | −3.1540504 | 5.04E−197 |
| SPP1 | 17.4107207 | 3.13518575 | 1.38E−198 | 1.98162896 | −3.1351858 | 1.38E−198 |

TABLE 3
Top underexpressed genes in glioblastoma relative to normal

| | | Glioblastoma | | | Normal | |
|---|---|---|---|---|---|---|
| Feature | Glioblastoma | Log2 Fold | Glioblastoma | Normal | Log2 Fold | Normal |
| Name | Average | Change | P-Value | Average | Change | P-Value |
| GABRA1 | 0 | −14.352183 | 0 | 1.03670324 | 14.3521834 | 0 |
| CCK | 0.00074358 | −11.865242 | 0 | 2.95899415 | 11.8652417 | 0 |
| SLC17A7 | 0.00039658 | −11.586549 | 0 | 1.37202871 | 11.5865489 | 0 |
| CHGA | 0.00104101 | −10.202205 | 0 | 1.28473428 | 10.2022052 | 0 |
| STMN2 | 0.00143759 | −9.8028619 | 0 | 1.32830594 | 9.80286189 | 0 |
| CALY | 0.00223075 | −9.0017361 | 0 | 1.168879 | 9.00173611 | 0 |
| EEF1A2 | 0.00307347 | −8.7977703 | 0 | 1.38980998 | 8.79777031 | 0 |
| CABP1 | 0.00257775 | −8.6400874 | 0 | 1.04813766 | 8.64008738 | 0 |
| NRGN | 0.0184904 | −8.0782977 | 0 | 5.01089186 | 8.07829775 | 0 |
| SNAP25 | 0.03618765 | −7.9643021 | 0 | 9.04995943 | 7.96430206 | 0 |
| ATP2B2 | 0.00475892 | −7.7395272 | 0 | 1.02758594 | 7.73952723 | 0 |
| SYN1 | 0.00758453 | −7.2364604 | 0 | 1.15114811 | 7.23646035 | 0 |
| NECAB1 | 0.00822897 | −6.9445903 | 0 | 1.01967756 | 6.94459029 | 0 |
| MBP | 0.06583178 | −6.8916979 | 0 | 7.82290092 | 6.8916979 | 0 |
| PHYHIP | 0.01090587 | −6.8331796 | 0 | 1.24912138 | 6.83317959 | 0 |
| BASP1 | 0.01913484 | −6.6459909 | 0 | 1.9212331 | 6.64599094 | 0 |
| CPLX1 | 0.01229389 | −6.505835 | 0 | 1.12168057 | 6.50583504 | 0 |
| VSNL1 | 0.04342518 | −6.505552 | 0 | 3.95001012 | 6.50555202 | 0 |
| TAGLN3 | 0.02201002 | −5.7653121 | 0 | 1.19980733 | 5.76531209 | 0 |
| ENC1 | 0.04322689 | −5.7367194 | 0 | 2.30763564 | 5.7367194 | 0 |
| FBXL16 | 0.02909884 | −5.2660351 | 0 | 1.12157983 | 5.26603506 | 0 |
| CHN1 | 0.1102484 | −5.2245807 | 0 | 4.12399452 | 5.22458072 | 0 |
| KIF5A | 0.04218588 | −5.1231949 | 0 | 1.47196648 | 5.12319487 | 0 |
| PLP1 | 0.12987896 | −5.0483235 | 0 | 4.2992886 | 5.04832352 | 0 |
| OLFM1 | 0.10008611 | −4.9282871 | 0 | 3.04890792 | 4.92828714 | 0 |
| SNCB | 0.05185244 | −4.838491 | 0 | 1.48491205 | 4.838491 | 0 |
| STXBP1 | 0.07178043 | −4.7219566 | 0 | 1.89559382 | 4.72195662 | 0 |
| ATP1B1 | 0.12283972 | −4.7048967 | 0 | 3.20495994 | 4.70489675 | 0 |
| DNM1 | 0.06513777 | −4.6966958 | 0 | 1.69042924 | 4.69669583 | 0 |
| SERPINI1 | 0.05502506 | −4.5038462 | 0 | 1.24947398 | 4.50384622 | 0 |
| PRKAR1B | 0.06434461 | −4.3941476 | 0 | 1.35394522 | 4.39414762 | 0 |
| MEF2C | 0.05338918 | −4.3447482 | 0 | 1.08576544 | 4.34474823 | 0 |
| MTURN | 0.06578221 | −4.266778 | 0 | 1.26720487 | 4.26677801 | 0 |
| NSF | 0.06107285 | −4.2061486 | 2.57E−308 | 1.12812817 | 4.20614862 | 2.57E−308 |
| SYT1 | 0.15193855 | −4.1835448 | 0 | 2.76168758 | 4.18354475 | 0 |
| MAP2 | 0.0757462 | −4.1097179 | 7.11E−302 | 1.3085098 | 4.10971787 | 7.11E−302 |
| MT-ATP8 | 0.16096068 | −4.0814395 | 1.19E−307 | 2.72572207 | 4.08143951 | 1.19E−307 |
| MAP1A | 0.09770665 | −4.0064399 | 1.21E−292 | 1.57104793 | 4.00643992 | 1.21E−292 |
| UCHL1 | 0.23298897 | −3.8907615 | 2.44E−285 | 3.45666813 | 3.89076146 | 2.44E−285 |
| FAIM2 | 0.08541277 | −3.8188601 | 2.44E−271 | 1.20600306 | 3.81886007 | 2.44E−271 |
| STMN1 | 0.21395328 | −3.6921083 | 1.89E−260 | 2.76596919 | 3.69210828 | 1.89E−260 |
| APLP1 | 0.1050929 | −3.5845635 | 1.70E−242 | 1.26131137 | 3.58456347 | 1.70E−242 |
| NCDN | 0.11808079 | −3.5523504 | 2.66E−241 | 1.3858306 | 3.55235042 | 2.66E−241 |
| STMN3 | 0.11198342 | −3.2329552 | 6.54E−204 | 1.05327559 | 3.2329552 | 6.54E−204 |
| MT-ND4L | 0.59382445 | −3.1987417 | 5.70E−207 | 5.45265307 | 3.19874171 | 5.70E−207 |
| BEX1 | 0.17335371 | −3.1870431 | 7.04E−201 | 1.57920817 | 3.18704311 | 7.04E−201 |
| MT-ND2 | 5.0047023 | −3.1332228 | 8.11E−201 | 43.9113161 | 3.13322283 | 8.11E−201 |
| PPP3CA | 0.20225426 | −3.1272765 | 5.75E−196 | 1.7676493 | 3.12727649 | 5.75E−196 |
| MT-ND5 | 1.25843789 | −3.0810993 | 1.52E−194 | 10.6500215 | 3.08109927 | 1.52E−194 |

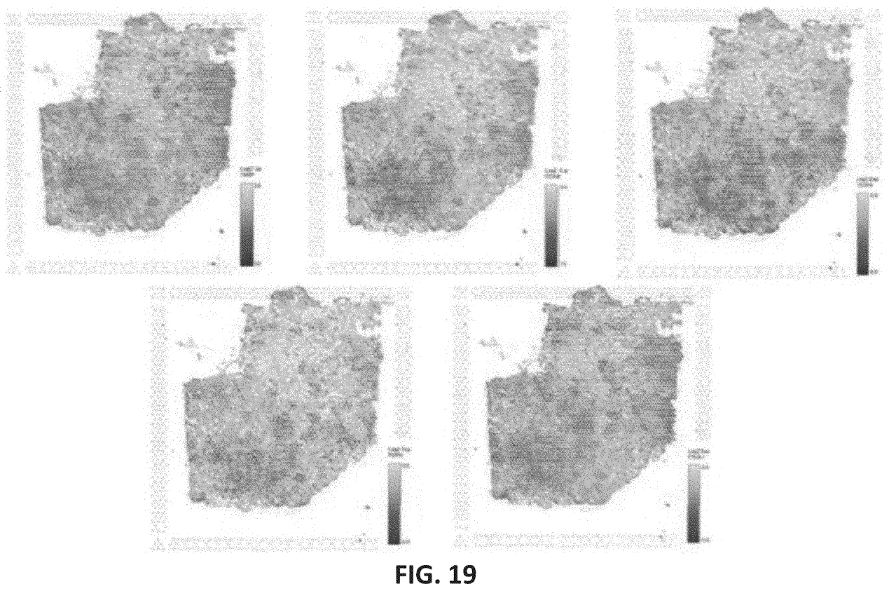

Spatial analysis of glioblastoma samples demonstrated regional distribution of overexpressed genes in glioblastoma (FIG. 19).

### Example 3: Identification of Differentially Expressed Genes in Glioblastoma

This example provides data from additional spatial analysis experiments that were performed to identify genes that are dysregulated in a glioblastoma tissue.

### Example 3A: Preparation of Biological Samples

In order to identify genes that are dysregulated in glioblastoma, fresh frozen samples from a subject diagnosed with glioblastoma were used herein. Normal (i.e., non-glioblastoma brain tissue) and glioblastoma samples were isolated, flash-frozen, and embedded in optimal cutting temperature compound (OCT). 10 μm sections were cut and placed on the capture area of a slide having arrays with 5000 spots with uniquely barcoded capture probes. A spot has an area of about 50 mm×50 mm on the slide. The capture probes are nucleic acid sequences having a spatial barcode, a unique molecule identifier, and a capture domain that includes a poly-thymine sequence in order to capture poly-adenylated sequences indiscriminately.

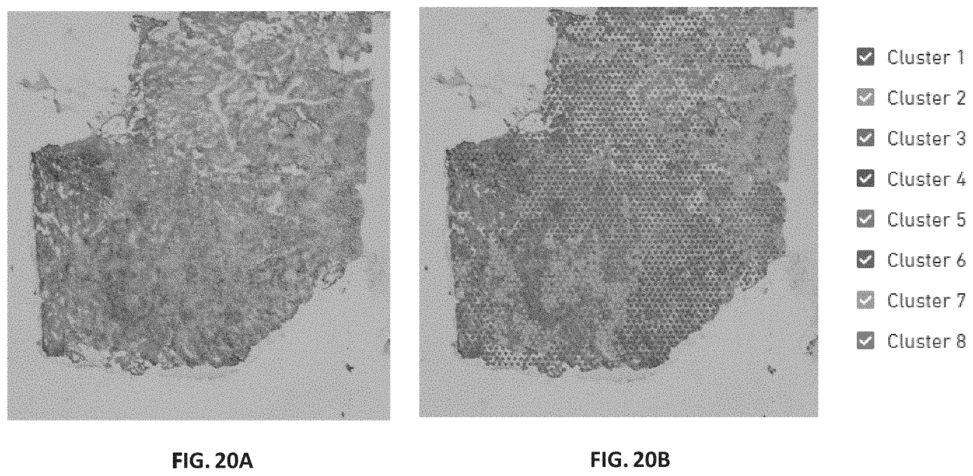

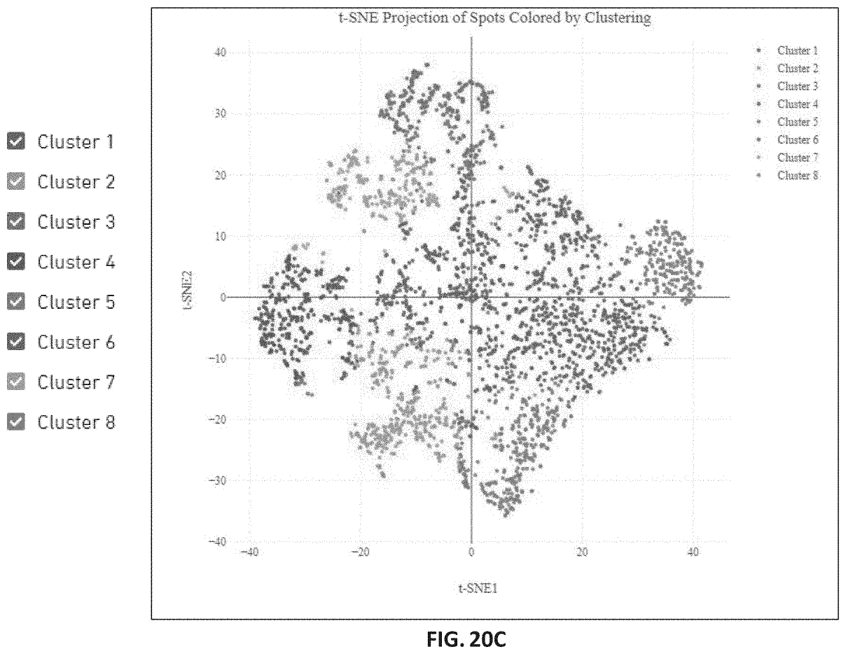

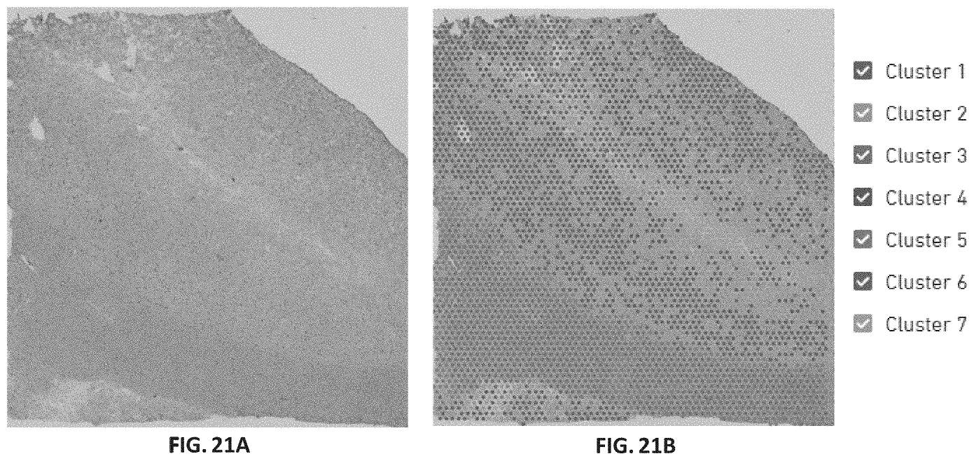

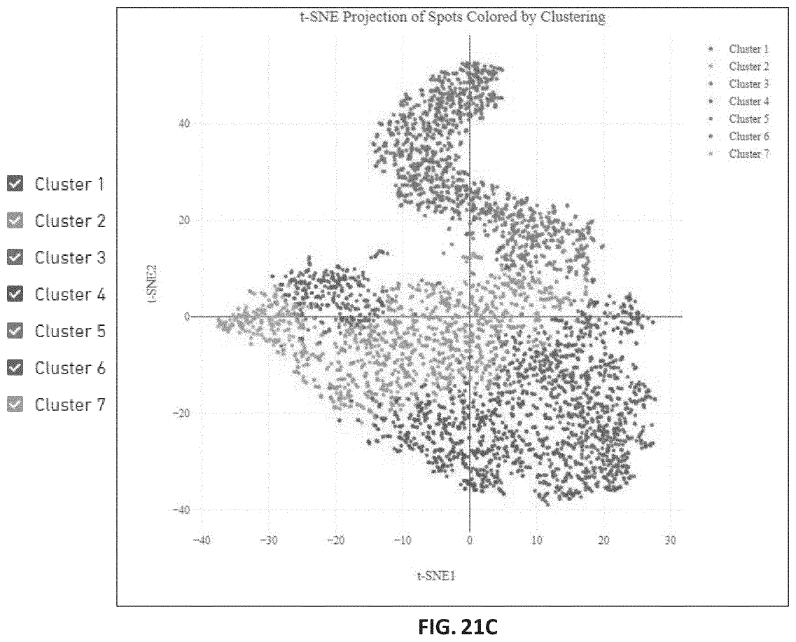

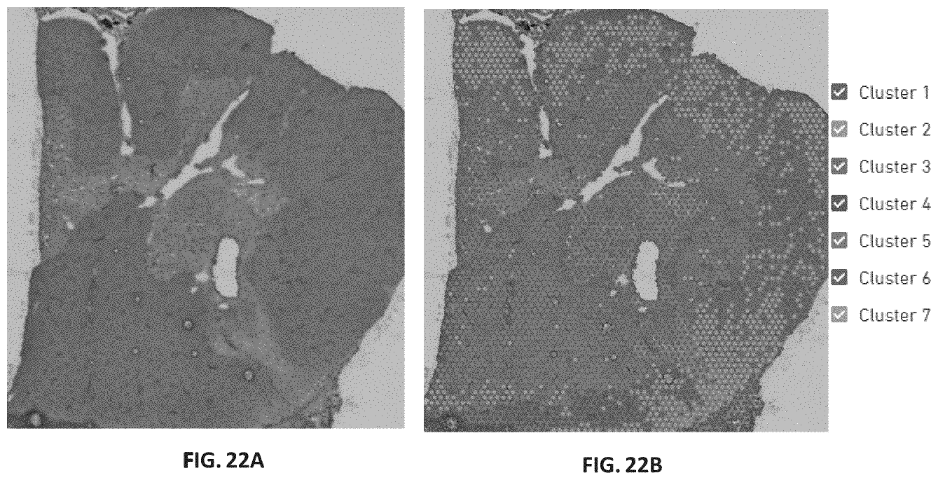

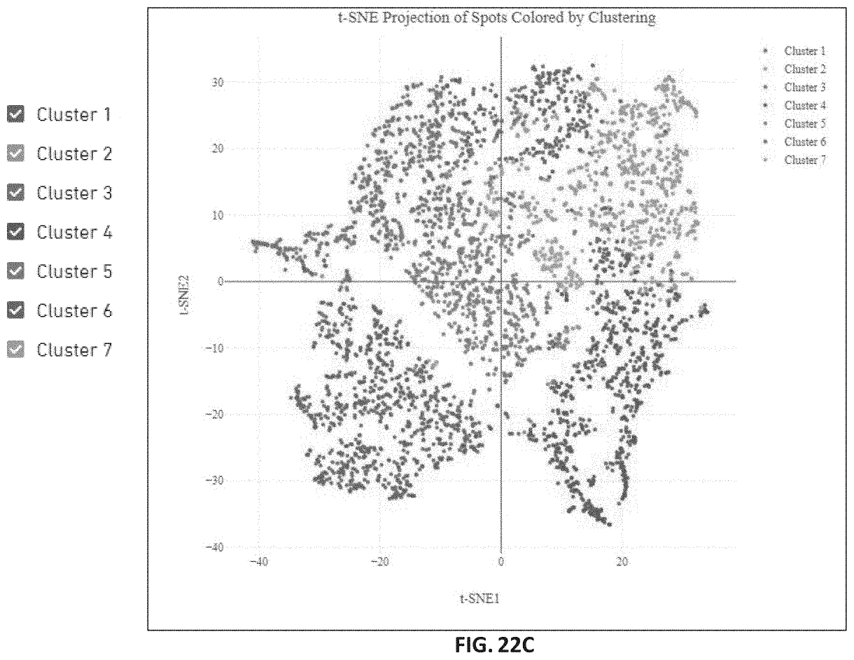

After placing the tissue section on the slide, each tissue was stained with hematoxylin and eosin (H&E) per established protocols. The stained tissues were imaged using brightfield microscopy. FIG. 20A shows a representative H&E stain image for a glioblastoma sample. FIGS. 21A and 22A show H&E stain images from representative normal samples. Fiducial markers were present on the slide within the frame surrounding the captured tissue allow for alignment of the spatial gene expression information (in Examples 3B and 3C) to the acquired image of the tissue section.

### Example 3B: Gene Detection in Normal and Glioblastoma Samples

The biological sample then was permeabilized to release analytes and was contacted with a plurality of capture probes attached to a slide. In particular, After 30 minutes, the tissues were washed and permeabilized by adding 1.25 mg/ml Proteinase K, incubated at 37° C. for at least 5 minutes and then washed to remove the protease. The released analytes then were allowed to hybridize to the capture domain on the capture probe immobilized on the spatial array via the polyA tail on each analyte (i.e., mRNA molecule). The captured analytes were copied, using the capture probe as a template and the extension product was released from the spatial array. Briefly, the tissues were incubated with a second strand extension mix comprising Kapa Hifi DNA polymerase (Roche) for 25 minutes at 53° C. Following incubation, the extension mix was removed from the tissues and the tissues were washed with SSC. A solution of KOH was added to each of the tissue wells, the tissues were incubated at room temperature for 10 minutes to release the extension product from the spatial array and the supernatant from each tissue well was transferred for quantitation, library preparation and sequencing on the Illumina NextSeq sequencing instrument.

### Example 3C: Analysis of Spatial Detection Results

The RNA-seq data were merged with the brightfield tissue images to align reads, perform clustering, and gene expression analysis. Additional analyses and data visualization were also performed. As shown in FIG. 20B, data clustering by gene expression profile overlaid on top of the H&E image demonstrates a loss of laminar organization in the tumor samples, which is preserved laminar organization of normal tissue (See FIGS. 21B and 22B). Further, visualization by t-SNE of gene expression measurements within each spot on the Visium array. In addition, t-SNE visualization of gene expression profiles glioblastoma (FIG. 20C) in healthy cerebrum demonstrating heterogeneity of the tissue. FIGS. 21C and 22C.

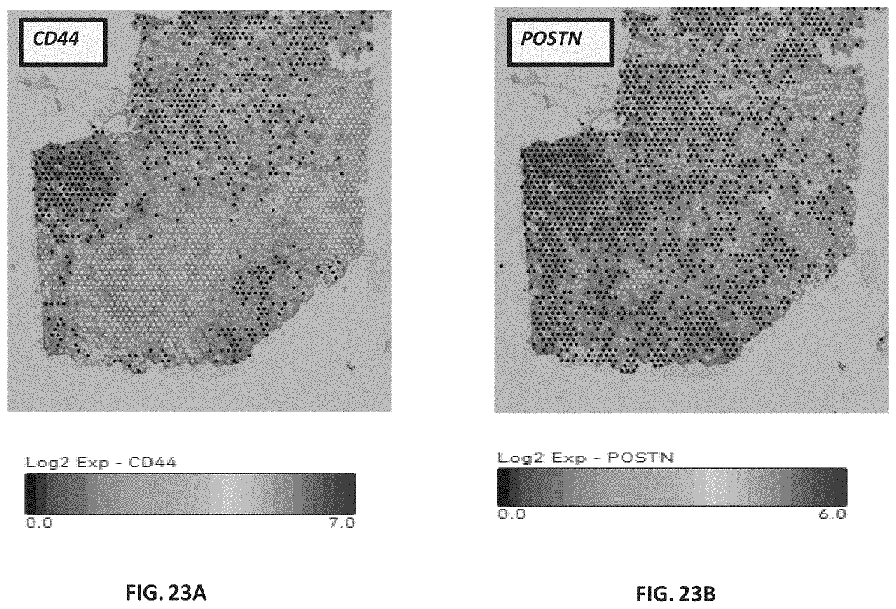

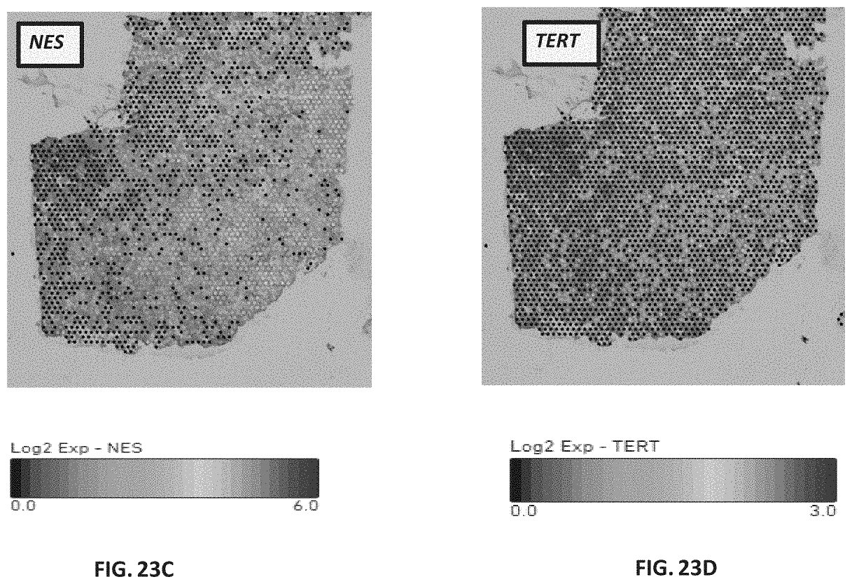

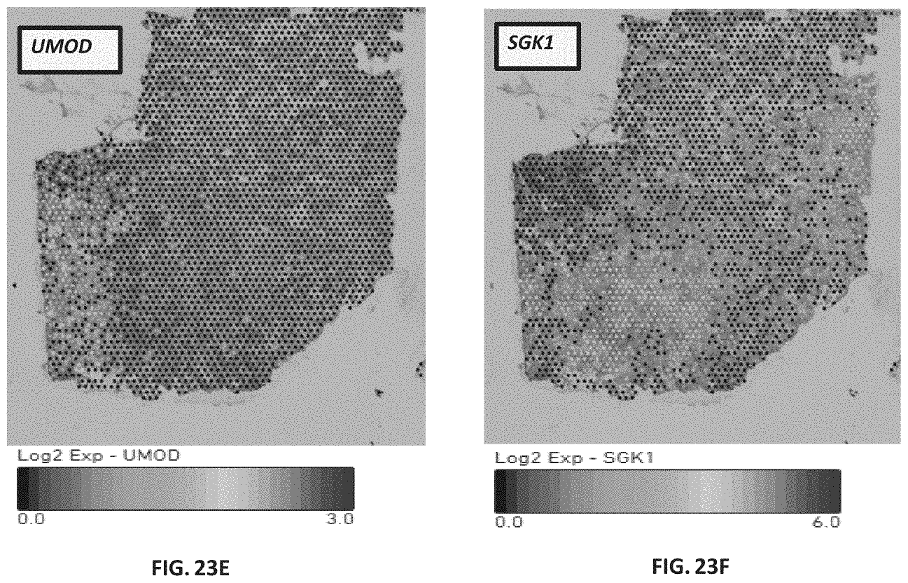

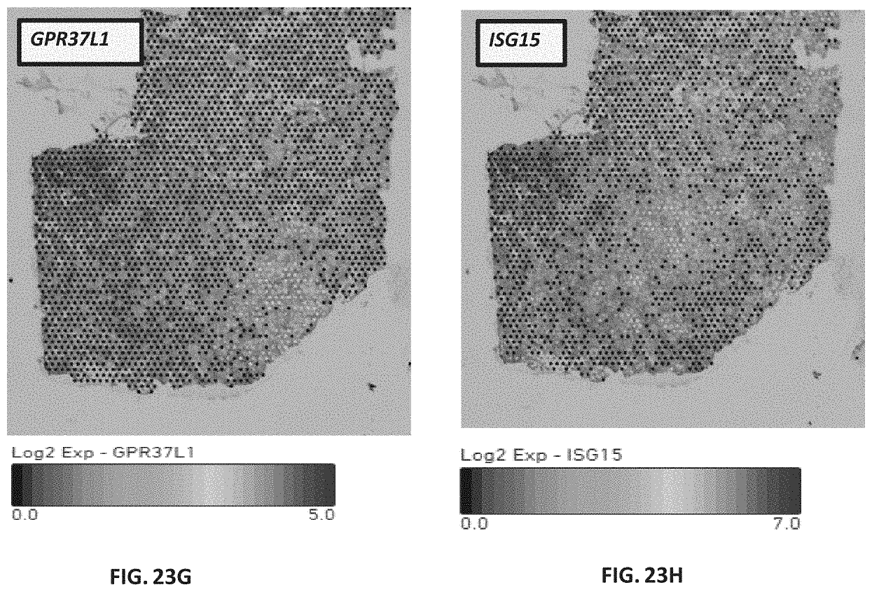

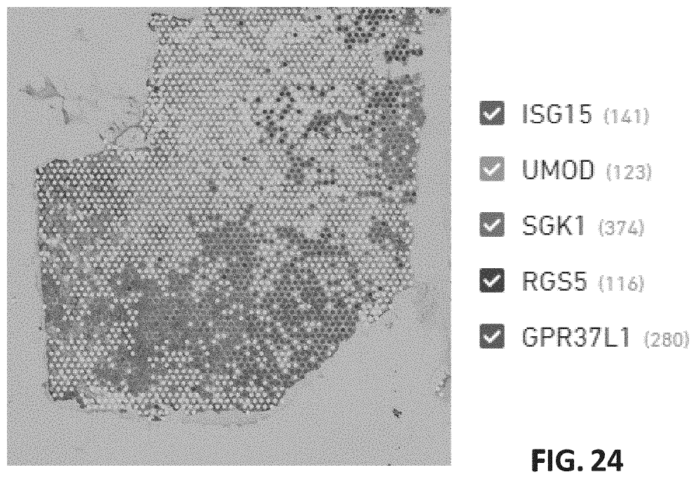

In addition to the clusters of genes identified as differentially expressed in glioblastoma samples as shown in FIG. 20B, individual genes that were differentially expressed in a glioblastoma sample were identified. In particular, CD44, periostin (POSTN), nestin (NES), telomerase reverse transcriptase (TERT), uromodulin (UMOD), serum/glucocorticoid regulated kinase 1 (SGK1), G protein-coupled receptor 37 like 1 (GPR37L1), ISG15 ubiquitin like modifier (ISG15), and regulator of G protein signaling 5 (RGS5) all were upregulated in a glioblastoma sample. See FIGS. 23A-23H and 24. In addition, spatial analysis provides the ability to demonstrate differentially-expressed genes in one image. See FIG. 24, which shows differential expression of ISG15, UMOD, SGK1, RGS5, and GPR37L1 in one image. Further, spatial analysis provided herein showed that certain genes are differentially expressed in certain spots. For example, as shown in FIG. 24, 141 out of 5000 spots showed overexpression of ISG15; 123 out of 5000 spots showed over expression of UMOD; 374 out of 5000 spots showed overexpression of SGK1; 116 out of 5000 spots showed overexpression of RGS5; and 280 out of 5000 spots showed overexpression of GPR37L1. Taken together, these data demonstrate that certain genes (e.g., CD44, POSTN, NES, TERT, UMOD, SGK1, GPR37L1, ISG1, and RGS5) are upregulated in the setting of glioblastoma. Further, this experiment is proof of concept that genes can be identified for both abundance and location in the setting of glioblastoma.

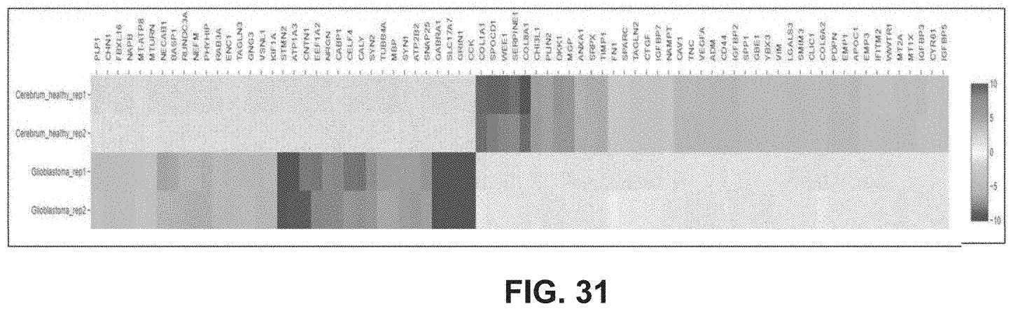

In this example, biomarkers for glioblastoma were identified. Exemplary dysregulated biomarkers are shown in Tables 4 and 5 below and in the heat map in FIG. 31.

TABLE 4
Biomarkers in healthy cerebrum

| | | | Cerebrum_healthy_rep 1 | | | Cerebrum_healthy_rep 2 | |
|---|---|---|---|---|---|---|---|
| | | Cerebrumhealthy_rep1 | Log2 Fold | Cerebrum_healthy_rep 1 | Cerebrum_healthy_rep 2 | Log2 Fold | Cerebrum_healthy_rep 2 |
| Feature ID | Biomarker | Average | Change | P-Value | Average | Change | P-Value |
| ENSG00000108821 | COL1A1 | 0.00165172 | −8.52365821 | 8.08E−194 | 0.00172304 | −8.44555616 | 6.14E−185 |
| ENSG00000134668 | SPOCD1 | 0.00188768 | −8.73028509 | 3.71E−221 | 0.00295379 | −8.12123246 | 3.95E−206 |
| ENSG00000166483 | WEE1 | 0.00353941 | −8.71051916 | 8.47E−256 | 0.00615373 | −7.93135597 | 3.67E−234 |
| ENSG00000106366 | SERPINE1 | 0.00896651 | −8.35291697 | 6.89E−283 | 0.01378436 | −7.72673915 | 1.22E−257 |
| ENSG00000144810 | COL8A1 | 0.00070788 | −10.0108015 | 1.89E−234 | 0.00246149 | −8.4725613 | 5.34E−214 |
| ENSG00000133048 | CHI3L1 | 0.12977844 | −6.95625135 | 5.76E−277 | 0.11716706 | −7.08666266 | 3.61E−271 |
| ENSG00000147872 | PLIN2 | 0.02359608 | −6.78476811 | 1.65E−238 | 0.02264573 | −6.82601785 | 7.70E−230 |
| ENSG00000107984 | DKK1 | 0.00448325 | −7.50704262 | 4.88E−205 | 0.00541528 | −7.22696328 | 2.11E−192 |
| ENSG00000111341 | MGP | 0.02548376 | −7.26405168 | 6.59E−266 | 0.02633797 | −7.1992713 | 7.24E−253 |
| ENSG00000135046 | ANXA1 | 0.04176506 | −6.01089108 | 3.38E−214 | 0.03864543 | −6.10546712 | 1.02E−208 |
| ENSG00000101955 | SRPX | 0.01226996 | −6.28600585 | 3.57E−189 | 0.01279976 | −6.20790379 | 1.66E−179 |
| ENSG00000102265 | TIMP1 | 0.09344047 | −6.30038671 | 1.65E−238 | 0.09058293 | −6.32817546 | 5.80E−230 |
| ENSG00000115414 | FN1 | 0.14912722 | −2.71218782 | 2.94E−64 | 0.12725918 | −2.934838 | 1.17E−69 |
| ENSG00000113140 | SPARC | 0.47593294 | −2.93208319 | 2.14E−83 | 0.40762321 | −3.14806157 | 9.59E−90 |
| ENSG00000158710 | TAGLN2 | 0.08824934 | −3.56858357 | 1.52E−102 | 0.09033678 | −3.51832137 | 1.30E−96 |
| ENSG00000118523 | CTGF | 0.08919318 | −3.34857391 | 3.40E−87 | 0.08049081 | −3.48446455 | 1.49E−88 |
| ENSG00000163453 | IGFBP7 | 0.52878816 | −3.62157439 | 1.69E−116 | 0.483191046 | −3.73865275 | 1.54E−117 |
| ENSG00000105835 | NAMPT | 0.12505922 | −3.2153357 | 2.13E−89 | 0.130459121 | −3.13723364 | 6.32E−83 |
| ENSG00000105974 | CAV1 | 0.03020298 | −5.20571565 | 7.33E−164 | 0.024368779 | −5.49751915 | 4.91E−166 |
| ENSG00000041982 | TNC | 0.01958474 | −5.18097156 | 7.30E−150 | 0.015507405 | −5.49791749 | 1.07E−151 |
| ENSG00000112715 | VEGFA | 0.12789075 | −5.2894767 | 1.63E−191 | 0.10682879 | −5.53342764 | 2.88E−194 |
| ENSG00000148926 | ADM | 0.05025965 | −5.46357434 | 2.56E−189 | 0.047752961 | −5.5204466 | 1.18E−183 |
| ENSG00000026508 | CD44 | 0.04672024 | −5.434406 | 6.54E−185 | 0.044060722 | −5.50199461 | 1.04E−179 |
| ENSG00000115457 | IGFBP2 | 0.09108087 | −5.64064671 | 8.90E−207 | 0.097228968 | −5.52918366 | 9.70E−194 |
| ENSG00000118785 | SPP1 | 0.55096848 | −4.82155706 | 4.40E−178 | 0.493037018 | −4.96670428 | 2.42E−178 |
| ENSG00000114480 | GBE1 | 0.03114682 | −4.83881361 | 4.81E−146 | 0.028553317 | −4.94737879 | 2.03E−143 |
| ENSG00000060138 | YBX3 | 0.04955176 | −4.82849109 | 5.26E−156 | 0.042829976 | −5.02277552 | 1.24E−156 |
| ENSG00000026025 | VIM | 0.91552792 | −4.78806956 | 7.28E−177 | 0.887122023 | −4.81746818 | 2.16E−171 |
| ENSG00000131981 | LGALS3 | 0.21614009 | −4.9892083 | 4.39E−186 | 0.221288207 | −4.93834602 | 2.11E−171 |
| ENSG00000256235 | SMIM3 | 0.02760741 | −4.99450452 | 1.09E−150 | 0.026584123 | −5.03185718 | 1.15E−145 |
| ENSG00000213719 | CLIC1 | 0.02760741 | −4.91333116 | 1.32E−146 | 0.028799466 | −4.83522911 | 3.25E−138 |
| ENSG00000142173 | COL6A2 | 0.02689953 | −4.88899705 | 1.37E−142 | 0.027076421 | −4.86245794 | 5.93E−136 |
| ENSG00000162493 | PDPN | 0.03020298 | −4.96381664 | 1.48E−151 | 0.022891883 | −5.34596889 | 9.69E−157 |
| ENSG00000134531 | EMP1 | 0.03091086 | −4.81728293 | 8.69E−145 | 0.021168839 | −5.34565813 | 2.00E−154 |
| ENSG00000185201 | IFITM2 | 0.07361977 | −4.26165374 | 1.45E−133 | 0.063752665 | −4.45487414 | 1.43E−135 |
| ENSG00000018408 | WWTR1 | 0.04672024 | −4.18603684 | 1.13E−122 | 0.033722452 | −4.64303082 | 7.54E−133 |
| ENSG00000125148 | MT2A | 2.10311866 | −4.50990104 | 1.70E−162 | 2.028270108 | −4.54643184 | 5.26E−158 |
| ENSG00000187193 | MT1X | 0.50873149 | −4.511623 | 6.75E−161 | 0.468914388 | −4.61412182 | 1.14E−159 |
| ENSG00000146674 | IGFBP3 | 0.03209067 | −4.90162652 | 1.48E−148 | 0.052183648 | −4.17909762 | 2.11E−118 |
| ENSG00000142871 | CYR61 | 0.09367644 | −4.01596094 | 2.91E−123 | 0.097228968 | −3.94525623 | 1.18E−115 |
| ENSG00000115461 | IGFBP5 | 0.19490362 | −3.86805597 | 1.85E−123 | 0.163196976 | −4.1122682 | 3.40E−129 |
| ENSG00000133110 | POSTN | 0.00306749 | −7.41526256 | 2.68E−180 | 0.004676836 | −6.8215414 | 1.42E−165 |
| ENSG00000132688 | NES | 0.03893353 | −4.15370168 | 1.72E−117 | 0.034953198 | −4.29400578 | 3.24E−117 |
| ENSG00000164362 | TERT | 0 | −8.15720339 | 3.49E−67 | 0.000246149 | −7.07764186 | 5.07E−62 |
| ENSG00000169344 | UMOD | 0 | −7.69722464 | 5.06E−49 | 0 | −7.61912259 | 1.18E−46 |
| ENSG00000118515 | SGK1 | 0.13732918 | −2.18524567 | 3.14E−46 | 0.122336195 | −2.34778194 | 1.08E−49 |
| ENSG00000187608 | ISG15 | 0.20198245 | −1.65435861 | 6.86E−30 | 0.198642473 | −1.66874059 | 3.23E−29 |
| ENSG00000232995 | RGS5 | 0.00165172 | −1.41683004 | 0.007789068 | 0.002461493 | −0.81959013 | 0.134049619 |

TABLE 5
Biomarkers in cerebrum of glioblastoma subjects.

| | | | Glioblastoma_rep1 | | | Glioblastoma_rep2 | |
|---|---|---|---|---|---|---|---|
| | | Glioblastoma_rep1 | Log2 Fold | Glioblastoma_rep1 | Glioblastoma_rep2 | Log2 Fold | Glioblastoma_rep2 |
| FeatureID | Biomarker | Average | Change | P-Value | Average | Change | P-Value |
| ENSG00000108821 | COL1A1 | 1.047265 | 1.701277 | 1.09E−52 | 0.858982 | 1.01141 | 1.69E−17 |
| ENSG00000134668 | SPOCD1 | 1.231537 | 1.38109 | 5.17E−36 | 1.261309 | 1.330997 | 1.35E−31 |
| ENSG00000166483 | WEE1 | 2.163478 | 1.386341 | 4.92E−37 | 2.206734 | 1.324516 | 6.29E−32 |
| ENSG00000106366 | SERPINE1 | 4.101244 | 1.374869 | 3.93E−37 | 4.213114 | 1.33365 | 5.19E−33 |
| ENSG00000144810 | COL8A1 | 1.307009 | 1.330347 | 2.08E−33 | 1.388348 | 1.385224 | 2.83E−34 |
| ENSG00000133048 | CHI3L1 | 22.20082 | 1.397138 | 1.48E−38 | 22.35111 | 1.298663 | 1.95E−31 |
| ENSG00000147872 | PLIN2 | 3.434338 | 1.244664 | 2.18E−30 | 3.839872 | 1.447512 | 5.56E−39 |
| ENSG00000107984 | DKK1 | 1.135257 | 1.282438 | 2.96E−30 | 1.240707 | 1.419634 | 4.10E−35 |
| ENSG00000111341 | MGP | 5.37087 | 1.36167 | 1.22E−36 | 5.549757 | 1.337763 | 2.02E−33 |
| ENSG00000135046 | ANXA1 | 3.731289 | 1.396355 | 1.57E−38 | 3.72596 | 1.275991 | 2.43E−30 |
| ENSG00000101955 | SRPX | 1.328699 | 1.363123 | 6.51E−35 | 1.361284 | 1.31584 | 1.01E−30 |
| ENSG00000102265 | TIMP1 | 10.33546 | 1.448368 | 2.75E−41 | 9.981202 | 1.23164 | 4.12E−28 |
| ENSG00000115414 | FN1 | 1.466418 | 1.539079 | 2.67E−43 | 1.118313 | 0.72306 | 3.08E−09 |
| ENSG00000113140 | SPARC | 5.104072 | 1.355562 | 2.56E−36 | 4.596051 | 0.977047 | 8.91E−18 |
| ENSG00000158710 | TAGLN2 | 1.474177 | 1.395076 | 4.27E−37 | 1.350378 | 1.051625 | 7.69E−20 |
| ENSG00000118523 | CTGF | 1.284791 | 1.396603 | 5.69E−35 | 1.163555 | 1.024463 | 9.55E−18 |
| ENSG00000163453 | IGFBP7 | 9.097045 | 1.381857 | 5.47E−38 | 8.492878 | 1.08848 | 3.62E−22 |
| ENSG00000105835 | NAMPT | 1.464831 | 1.06025 | 1.10E−21 | 1.665856 | 1.312928 | 2.43E−31 |
| ENSG00000105974 | CAV1 | 1.558289 | 1.407583 | 9.29E−38 | 1.526092 | 1.233956 | 2.43E−27 |
| ENSG00000041982 | TNC | 0.95363 | 1.272374 | 1.38E−29 | 1.027022 | 1.369194 | 2.13E−32 |
| ENSG00000112715 | VEGFA | 6.597822 | 1.252288 | 5.64E−31 | 7.215008 | 1.3925 | 2.91E−36 |
| ENSG00000148926 | ADM | 2.96969 | 1.290543 | 1.51E−32 | 3.166702 | 1.358862 | 3.95E−34 |
| ENSG00000026508 | CD44 | 2.817158 | 1.411342 | 8.26E−39 | 2.758317 | 1.236552 | 4.55E−28 |
| ENSG00000115457 | IGFBP2 | 6.100552 | 1.306702 | 7.93E−34 | 6.442065 | 1.347018 | 5.48E−34 |
| ENSG00000118785 | SPP1 | 21.3253 | 1.357918 | 7.58E−37 | 21.38973 | 1.254147 | 1.23E−29 |
| ENSG00000114480 | GBE1 | 1.19239 | 1.269222 | 5.96E−30 | 1.273831 | 1.344012 | 9.71E−32 |
| ENSG00000060138 | YBX3 | 1.8697 | 1.255594 | 2.20E−30 | 2.018094 | 1.359511 | 1.09E−33 |
| ENSG00000026025 | VIM | 34.03267 | 1.306202 | 3.50E−34 | 35.31906 | 1.299277 | 6.20E−32 |
| ENSG00000131981 | LGALS3 | 9.388706 | 1.35675 | 1.22E−36 | 9.444161 | 1.260777 | 7.76E−30 |
| ENSG00000256235 | SMIM3 | 1.214256 | 1.361396 | 1.78E−34 | 1.219299 | 1.260265 | 7.30E−28 |
| ENSG00000213719 | CLIC1 | 1.171759 | 1.423048 | 7.85E−38 | 1.122151 | 1.188347 | 7.72E−25 |
| ENSG00000142173 | COL6A2 | 1.190274 | 1.609591 | 8.38E−49 | 0.998544 | 1.000227 | 1.03E−17 |
| ENSG00000162493 | PDPN | 1.287436 | 1.332658 | 1.60E−33 | 1.323314 | 1.297297 | 5.51E−30 |
| ENSG00000134531 | EMP1 | 1.212669 | 1.386481 | 4.22E−36 | 1.197486 | 1.237679 | 4.10E−27 |
| ENSG00000185201 | IFITM2 | 1.96598 | 1.386766 | 2.84E−37 | 1.896709 | 1.176858 | 2.86E−25 |
| ENSG00000018408 | WWTR1 | 1.175638 | 1.357557 | 2.04E−34 | 1.160121 | 1.210783 | 8.55E−26 |
| ENSG00000125148 | MT2A | 63.52167 | 1.256583 | 1.15E−31 | 67.68511 | 1.325115 | 2.80E−33 |
| ENSG00000187193 | MT1X | 15.34378 | 1.248028 | 6.50E−31 | 16.46831 | 1.336869 | 1.56E−33 |
| ENSG00000146674 | IGFBP3 | 1.275974 | 1.254591 | 1.47E−29 | 1.36088 | 1.325835 | 3.46E−31 |
| ENSG00000142871 | CYR61 | 2.01606 | 1.245089 | 2.14E−29 | 2.116252 | 1.273998 | 4.91E−29 |
| ENSG00000115461 | IGFBP5 | 3.723354 | 1.195291 | 3.06E−28 | 4.050729 | 1.323914 | 9.92E−33 |
| ENSG00000133110 | POSTN | 0.80392 | 1.513073 | 1.38E−41 | 0.747292 | 1.185242 | 5.23E−24 |
| ENSG00000132688 | NES | 0.964034 | 1.37263 | 4.35E−35 | 0.934318 | 1.176706 | 2.78E−24 |
| ENSG00000164362 | TERT | 0.091166 | 1.35854 | 3.73E−21 | 0.094926 | 1.356442 | 3.44E−20 |
| ENSG00000169344 | UMOD | 0.069477 | 1.505787 | 2.86E−19 | 0.065439 | 1.215037 | 3.67E−12 |
| ENSG00000118515 | SGK1 | 0.811326 | 1.057309 | 1.93E−20 | 0.825455 | 1.0217 | 6.08E−18 |
| ENSG00000187608 | ISG15 | 0.81256 | 0.944948 | 7.17E−17 | 0.793746 | 0.82242 | 4.42E−12 |
| ENSG00000232995 | RGS5 | 0.005643 | 0.654059 | 0.155763 | 0.006261 | 0.852593 | 0.048621 |

### Example 4: Identification of Differentially Expressed Genes in Glioblastoma Combined with Immunofluorescence

This example provides data from additional spatial analysis experiments that were performed to identify genes that are dysregulated in a glioblastoma tissue co-localized with protein detection using immunofluorescence.

### Example 4A: Preparation of Biological Samples

Preparation of the biological samples were performed in a similar manner as in Example 3 with the following changes. Instead of staining with H&E, samples were stained for detection of proteins of interest using immunofluorescence, and then imaged on a fluorescent microscope.

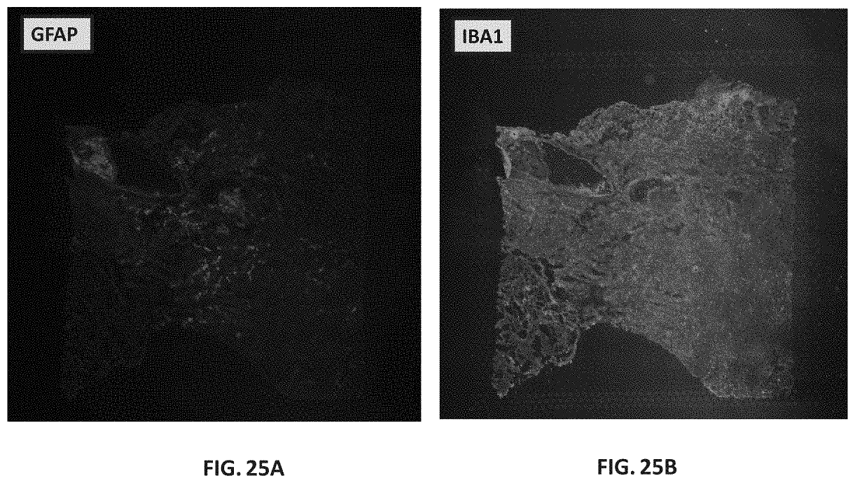

Briefly, after placing the tissue section on the slide, each tissue was fixed with MeOH for 30 minutes at −20° C. After a series of washes in 1×PBS, the samples were blocked using blocking buffer at room temperature for 5 minutes. Blocking buffer was removed, and then the samples were incubated with a primary antibody that was conjugated with a fluorophore at room temperature for 30 minutes. The samples then were washed using standard wash buffer (e.g., lx PBS) four times for 5-10 minutes per wash. Optionally, samples are counterstained with DAPI. After, samples are immersed in 3×SSC buffer and a cover slip was applied. Samples were then imaged at 20× under a fluorescent microscope. FIGS. 25A-25B show representative images of immunofluorescent detection of glial fibrillary acidic protein (GFAP; GFAP-Alexa 647, clone 644704), and astrocyte marker; and ionized calcium-binding adaptor molecule 1 (IBA1; clone EPR16588), a microglia marker. The tissue section was also stained against an astrocyte marker, glial fibrillary acidic protein.

Once again, fiducial markers were present on the slide within the frame surrounding the captured tissue allow for alignment of the spatial gene expression information (in Examples 4B and 4C) to the acquired image of the tissue section.

### Example 4B: Gene Detection and Analysis of Normal and Glioblastoma Samples

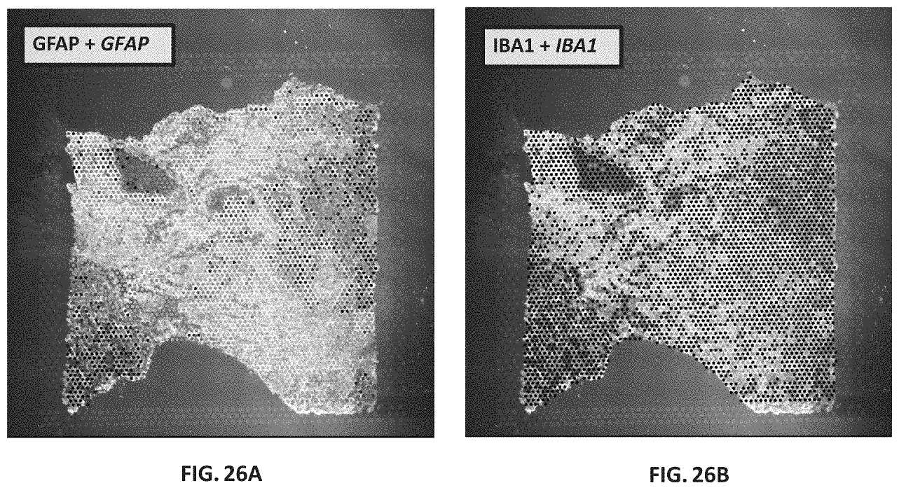

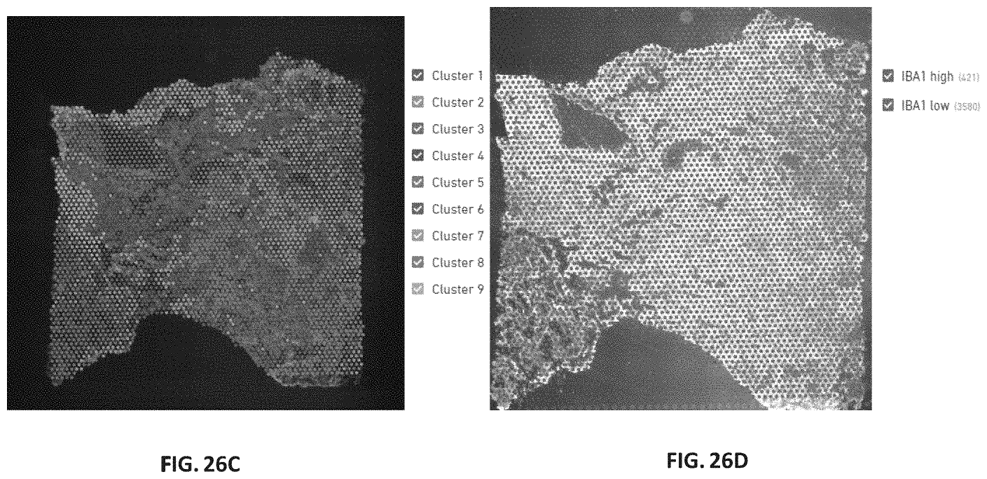

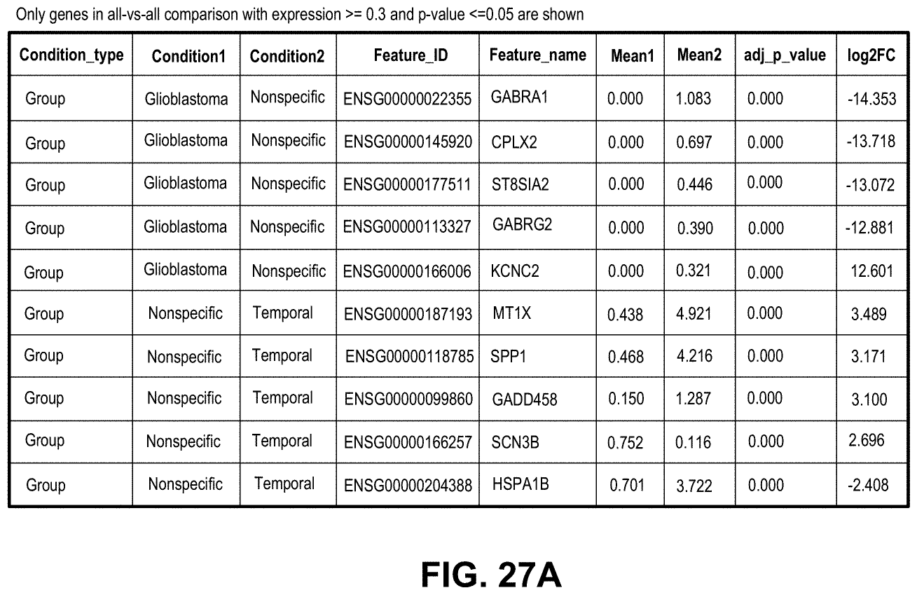

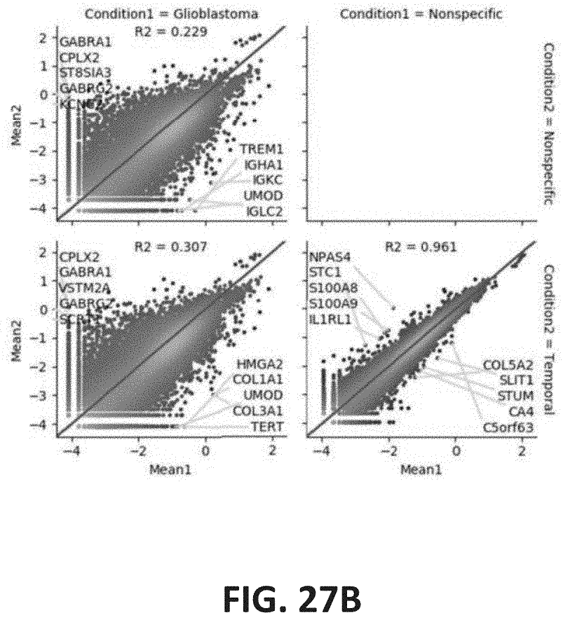

After imaging, the biological sample then immersed in 3× in SSC buffer, and permeabilized using similar methods disclosed in Example 3B. Analytes were detected and analyzed using similar methods disclosed in Examples 3B and 3C, and both individual genes and clusters were identified in each spot. As shown in FIGS. 26A-26B, both protein and mRNA expression can be detected and imaged. FIG. 26A shows protein expression of GFAP and mRNA expression of GEAP. FIG. 26B shows protein expression of IBA1 and mRNA expression of IBA1. Thus, combining gene expression data with immunostaining allows for simultaneous detection of mRNA and protein. Alternatively, protein expression can be combined with cluster expression to overlap for simultaneous detection of mRNA clusters and protein. See, e.g., FIG. 26C. Finally, based on expression abundance of any particular mRNA, spots can be bifurcated as spots that express a particular mRNA of interest at a high abundance or at a low abundance. For example, as shown in FIG. 26D, 421 spots in the glioblastoma sample express IBA1 at a high abundance, and 3580 express IBA1 at low abundance. These data demonstrate the power of co-localizing protein and mRNA expression in a single sample using spatial detection and immunofluorescence.

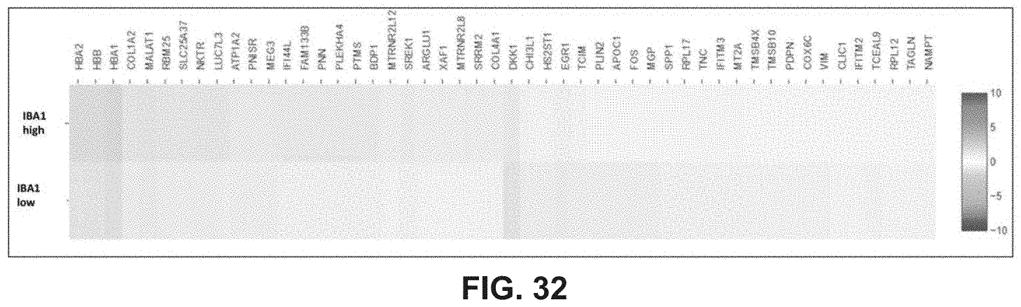

In this example, biomarkers that associated with differential expression of IBA1 in a glioblastoma sample were identified. Exemplary dysregulated biomarkers that associated with differential expression of IBA1 are shown in the heat map in FIG. 32.

### Example 5: Comparison Between Healthy and Glioblastoma Samples

Spatial analysis was performed on healthy (nonspecific & temporal) and glioblastoma samples from different patients. Two tissue sections each taken from a glioblastoma sample and a healthy control sample were used. A scatter plot demonstrated the differential expression of genes captured using the methods described herein (FIG. 26B). Decreased expression of GABRA CPLX2, ST8SIA3, GABRG2, and KCNC2 was observed in glioblastoma compared to healthy sample (FIG. 26A).

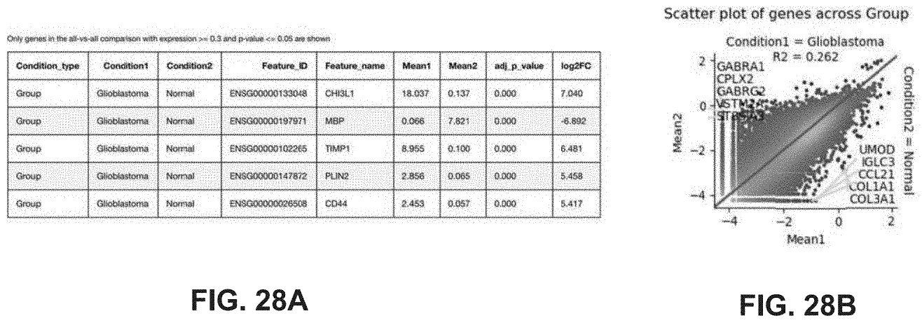

A scatter plot demonstrated the differential expression of genes captured using the methods described herein (FIG. 28B). Differentially expressed genes were observed. For example, overexpression of CHI3L1, TIMP1, PLIN2, and CD44 and underexpression of MBP within the glioblastoma sample were observed (FIG. 28A).

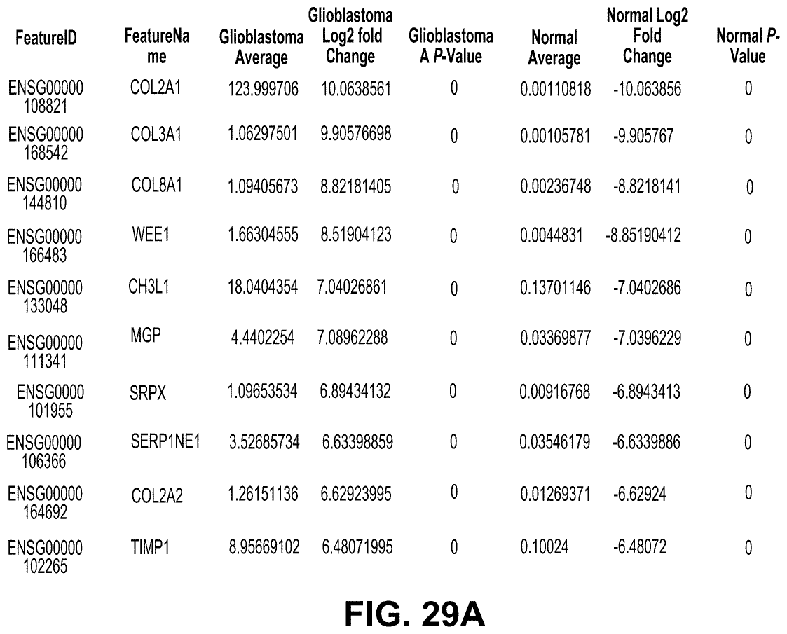

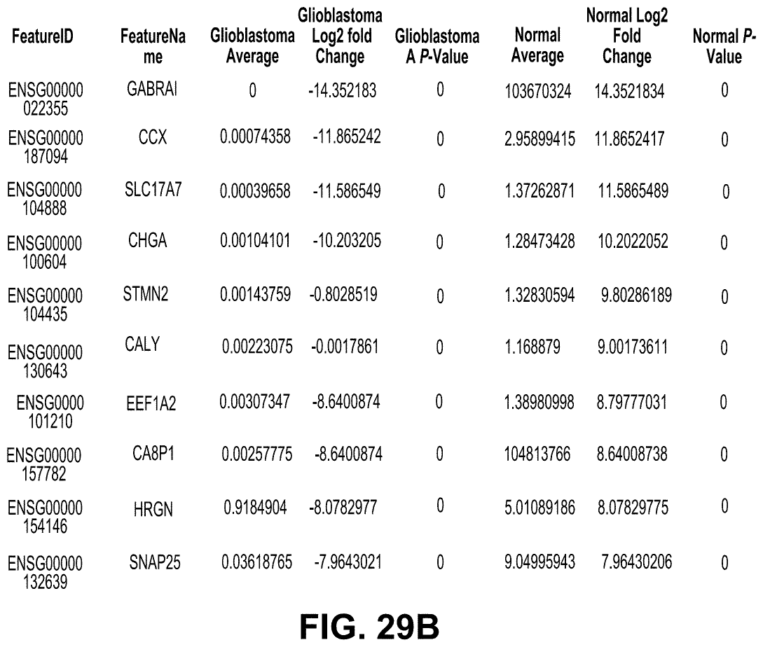

Comparison of between healthy and glioblastoma samples also showed other genes were differentially expressed, for example, the genes shown in FIGS. 29A and 29B as well as Tables 6 and 7.

TABLE 6
Top overexpressed genes in glioblastoma relative to normal

| | | Glioblastoma | | | Normal | |
|---|---|---|---|---|---|---|
| Feature | Glioblastoma | Log2 Fold | Glioblastoma | Normal | Log2 Fold | Normal |
| Name | Average | Change | P-Value | Average | Change | P-Value |
| COL1A1 | 1.23999706 | 10.0638561 | 0 | 0.00110818 | −10.063856 | 0 |
| COL3A1 | 1.06297501 | 9.90576698 | 0 | 0.00105781 | −9.905767 | 0 |
| COL8A1 | 1.09405673 | 8.82181405 | 0 | 0.00236748 | −8.8218141 | 0 |
| WEE1 | 1.66304555 | 8.51904123 | 0 | 0.0044831 | −8.5190412 | 0 |
| CHI3L1 | 18.0404354 | 7.04026861 | 0 | 0.13701146 | −7.0402686 | 0 |
| MGP | 4.4401254 | 7.03962288 | 0 | 0.03369877 | −7.0396229 | 0 |
| SRPX | 1.09653534 | 6.89434132 | 0 | 0.00916768 | −6.8943413 | 0 |
| SERPINE1 | 3.52695734 | 6.63398859 | 0 | 0.03546179 | −6.6339886 | 0 |
| COL1A2 | 1.26151136 | 6.62923995 | 0 | 0.01269371 | −6.62924 | 0 |
| TIMP1 | 8.95669102 | 6.48071995 | 0 | 0.10024 | −6.48072 | 0 |
| ANXA1 | 3.15377841 | 6.32864809 | 0 | 0.03918931 | −6.3286481 | 0 |
| COL6A2 | 1.06297501 | 5.85344594 | 0 | 0.01833536 | −5.8534459 | 0 |
| CAV1 | 1.20608973 | 5.56157824 | 0 | 0.02548816 | −5.5615782 | 0 |
| PLIN2 | 2.85624653 | 5.45801383 | 0 | 0.06492933 | −5.4580138 | 0 |
| CD44 | 2.4537209 | 5.41720583 | 0 | 0.05737355 | −5.4172058 | 0 |
| APOC1 | 5.78253847 | 5.3730687 | 0 | 0.13947968 | −5.3730687 | 0 |
| IGFBP2 | 4.66914861 | 5.36192666 | 0 | 0.1134878 | −5.3619267 | 0 |
| PDPN | 1.03333089 | 5.35572835 | 0 | 0.02518593 | −5.3557283 | 0 |
| VIM | 25.9392573 | 5.23652741 | 0 | 0.68797886 | −5.2365274 | 0 |
| LGALS3 | 6.94574832 | 4.90982887 | 0 | 0.23100535 | −4.9098289 | 0 |
| VEGFA | 5.15530241 | 4.75663587 | 0 | 0.19065749 | −4.7566359 | 0 |
| IGFBP5 | 2.99698179 | 4.62036276 | 0 | 0.12179916 | −4.6203628 | 0 |
| CTGF | 1.18611217 | 4.55321848 | 0 | 0.0504726 | −4.5532185 | 0 |
| EMP1 | 1.07288944 | 4.35483735 | 0 | 0.05238673 | −4.3548374 | 0 |
| EMP3 | 1.07353388 | 4.35155197 | 0 | 0.05253785 | −4.351552 | 0 |
| IGFBP3 | 1.07958168 | 4.28689993 | 0 | 0.05525793 | −4.2868999 | 0 |
| A2M | 4.80264633 | 4.26503459 | 0 | 0.24974368 | −4.2650346 | 0 |
| ANXA2 | 1.02544892 | 4.21795198 | 0 | 0.05505644 | −4.217952 | 0 |
| FLNA | 1.36933073 | 4.14183216 | 3.01E−306 | 0.07752229 | −4.1418322 | 3.01E−306 |
| IGFBP7 | 7.97159381 | 4.12851759 | 0 | 0.45571421 | −4.1285176 | 0 |
| S100A11 | 1.60574018 | 4.0907128 | 1.96E−302 | 0.09419538 | −4.0907128 | 1.96E−302 |
| ADM | 2.30331908 | 4.0587042 | 1.38E−299 | 0.13817001 | −4.0587042 | 1.38E−299 |
| FN1 | 1.64138253 | 4.03850975 | 6.14E−292 | 0.09983703 | −4.0385097 | 6.14E−292 |
| SERPING1 | 2.00553934 | 3.98757791 | 1.69E−291 | 0.126383 | −3.9875779 | 1.69E−291 |
| MT2A | 47.8913793 | 3.97755744 | 2.62E−295 | 3.04009285 | −3.9775574 | 2.62E−295 |
| S100A10 | 2.19525186 | 3.78345289 | 3.35E−266 | 0.15937656 | −3.7834529 | 3.35E−266 |
| SPARC | 5.0779699 | 3.77129812 | 8.99E−269 | 0.37184507 | −3.7712981 | 8.99E−269 |
| ITGB1 | 1.24768074 | 3.72288566 | 3.20E−257 | 0.09444724 | −3.7228857 | 3.20E−257 |
| SLC5A3 | 1.06183486 | 3.7215987 | 2.00E−255 | 0.08044386 | −3.7215987 | 2.00E−255 |
| FABP7 | 1.92766154 | 3.62028615 | 1.98E−247 | 0.15670686 | −3.6202862 | 1.98E−247 |
| YBX3 | 1.44121031 | 3.58458058 | 3.83E−242 | 0.12008651 | −3.5845806 | 3.83E−242 |
| IFITM2 | 1.64420815 | 3.57344722 | 8.11E−243 | 0.13806927 | −3.5734472 | 8.11E−243 |
| TAGLN2 | 1.1312854 | 3.55871738 | 6.46E−238 | 0.09595839 | −3.5587174 | 6.46E−238 |
| COL6A1 | 1.06917153 | 3.55021661 | 4.43E−238 | 0.09122344 | −3.5502166 | 4.43E−238 |
| HLA-A | 5.10359869 | 3.51334628 | 4.82E−240 | 0.44689914 | −3.5133463 | 4.82E−240 |
| LGALS3BP | 1.08384488 | 3.31576455 | 1.13E−211 | 0.10880322 | −3.3157645 | 1.13E−211 |
| ANXA5 | 1.56201757 | 3.29340226 | 4.49E−211 | 0.15927582 | −3.2934023 | 4.49E−211 |
| APOE | 23.4428053 | 3.2832261 | 4.26E−215 | 2.40797638 | −3.2832261 | 4.26E−215 |
| GADD45A | 1.28461197 | 3.24716836 | 8.61E−204 | 0.13524844 | −3.2471684 | 8.61E−204 |
| TPM4 | 1.83877873 | 3.15405038 | 5.04E−197 | 0.20652462 | −3.1540504 | 5.04E−197 |
| SPP1 | 17.4107207 | 3.13518575 | 1.38E−198 | 1.98162896 | −3.1351858 | 1.38E−198 |

TABLE 7
Top underexpressed genes in glioblastoma relative to normal

| | | Glioblastoma | | | Normal | |
|---|---|---|---|---|---|---|
| Feature | Glioblastoma | Log2 Fold | Glioblastoma | Normal | Log2 Fold | Normal |
| Name | Average | Change | P-Value | Average | Change | P-Value |
| GABRA1 | 0 | −14.352183 | 0 | 1.03670324 | 14.3521834 | 0 |
| CCK | 0.00074358 | −11.865242 | 0 | 2.95899415 | 11.8652417 | 0 |
| SLC17A7 | 0.00039658 | −11.586549 | 0 | 1.37202871 | 11.5865489 | 0 |
| CHGA | 0.00104101 | −10.202205 | 0 | 1.28473428 | 10.2022052 | 0 |
| STMN2 | 0.00143759 | −9.8028619 | 0 | 1.32830594 | 9.80286189 | 0 |
| CALY | 0.00223075 | −9.0017361 | 0 | 1.168879 | 9.00173611 | 0 |
| EEF1A2 | 0.00307347 | −8.7977703 | 0 | 1.38980998 | 8.79777031 | 0 |
| CABP1 | 0.00257775 | −8.6400874 | 0 | 1.04813766 | 8.64008738 | 0 |
| NRGN | 0.0184904 | −8.0782977 | 0 | 5.01089186 | 8.07829775 | 0 |
| SNAP25 | 0.03618765 | −7.9643021 | 0 | 9.04995943 | 7.96430206 | 0 |
| ATP2B2 | 0.00475892 | −7.7395272 | 0 | 1.02758594 | 7.73952723 | 0 |
| SYN1 | 0.00758453 | −7.2364604 | 0 | 1.15114811 | 7.23646035 | 0 |
| NECAB1 | 0.00822897 | −6.9445903 | 0 | 1.01967756 | 6.94459029 | 0 |
| MBP | 0.06583178 | −6.8916979 | 0 | 7.82290092 | 6.8916979 | 0 |
| PHYHIP | 0.01090587 | −6.8331796 | 0 | 1.24912138 | 6.83317959 | 0 |
| BASP1 | 0.01913484 | −6.6459909 | 0 | 1.9212331 | 6.64599094 | 0 |
| CPLX1 | 0.01229389 | −6.505835 | 0 | 1.12168057 | 6.50583504 | 0 |
| VSNL1 | 0.04342518 | −6.505552 | 0 | 3.95001012 | 6.50555202 | 0 |
| TAGLN3 | 0.02201002 | −5.7653121 | 0 | 1.19980733 | 5.76531209 | 0 |
| ENC1 | 0.04322689 | −5.7367194 | 0 | 2.30763564 | 5.7367194 | 0 |
| FBXL16 | 0.02909884 | −5.2660351 | 0 | 1.12157983 | 5.26603506 | 0 |
| CHN1 | 0.1102484 | −5.2245807 | 0 | 4.12399452 | 5.22458072 | 0 |
| KIF5A | 0.04218588 | −5.1231949 | 0 | 1.47196648 | 5.12319487 | 0 |
| PLP1 | 0.12987896 | −5.0483235 | 0 | 4.2992886 | 5.04832352 | 0 |
| OLFM1 | 0.10008611 | −4.9282871 | 0 | 3.04890792 | 4.92828714 | 0 |
| SNCB | 0.05185244 | −4.838491 | 0 | 1.48491205 | 4.838491 | 0 |
| STXBP1 | 0.07178043 | −4.7219566 | 0 | 1.89559382 | 4.72195662 | 0 |
| ATP1B1 | 0.12283972 | −4.7048967 | 0 | 3.20495994 | 4.70489675 | 0 |
| DNM1 | 0.06513777 | −4.6966958 | 0 | 1.69042924 | 4.69669583 | 0 |
| SERPINI1 | 0.05502506 | −4.5038462 | 0 | 1.24947398 | 4.50384622 | 0 |
| PRKAR1B | 0.06434461 | −4.3941476 | 0 | 1.35394522 | 4.39414762 | 0 |
| MEF2C | 0.05338918 | −4.3447482 | 0 | 1.08576544 | 4.34474823 | 0 |
| MTURN | 0.06578221 | −4.266778 | 0 | 1.26720487 | 4.26677801 | 0 |
| NSF | 0.06107285 | −4.2061486 | 2.57E−308 | 1.12812817 | 4.20614862 | 2.57E−308 |
| SYT1 | 0.15193855 | −4.1835448 | 0 | 2.76168758 | 4.18354475 | 0 |
| MAP2 | 0.0757462 | −4.1097179 | 7.11E−302 | 1.3085098 | 4.10971787 | 7.11E−302 |
| MT-ATP8 | 0.16096068 | −4.0814395 | 1.19E−307 | 2.72572207 | 4.08143951 | 1.19E−307 |
| MAP1A | 0.09770665 | −4.0064399 | 1.21E−292 | 1.57104793 | 4.00643992 | 1.21E−292 |
| UCHL1 | 0.23298897 | −3.8907615 | 2.44E−285 | 3.45666813 | 3.89076146 | 2.44E−285 |
| FAIM2 | 0.08541277 | −3.8188601 | 2.44E−271 | 1.20600306 | 3.81886007 | 2.44E−271 |
| STMN1 | 0.21395328 | −3.6921083 | 1.89E−260 | 2.76596919 | 3.69210828 | 1.89E−260 |
| APLP1 | 0.1050929 | −3.5845635 | 1.70E−242 | 1.26131137 | 3.58456347 | 1.70E−242 |
| NCDN | 0.11808079 | −3.5523504 | 2.66E−241 | 1.3858306 | 3.55235042 | 2.66E−241 |
| STMN3 | 0.11198342 | −3.2329552 | 6.54E−204 | 1.05327559 | 3.2329552 | 6.54E−204 |
| MT-ND4L | 0.59382445 | −3.1987417 | 5.70E−207 | 5.45265307 | 3.19874171 | 5.70E−207 |
| BEX1 | 0.17335371 | −3.1870431 | 7.04E−201 | 1.57920817 | 3.18704311 | 7.04E−201 |
| MT-ND2 | 5.0047023 | −3.1332228 | 8.11E−201 | 43.9113161 | 3.13322283 | 8.11E−201 |
| PPP3CA | 0.20225426 | −3.1272765 | 5.75E−196 | 1.7676493 | 3.12727649 | 5.75E−196 |
| MT-ND5 | 1.25843789 | −3.0810993 | 1.52E−194 | 10.6500215 | 3.08109927 | 1.52E−194 |

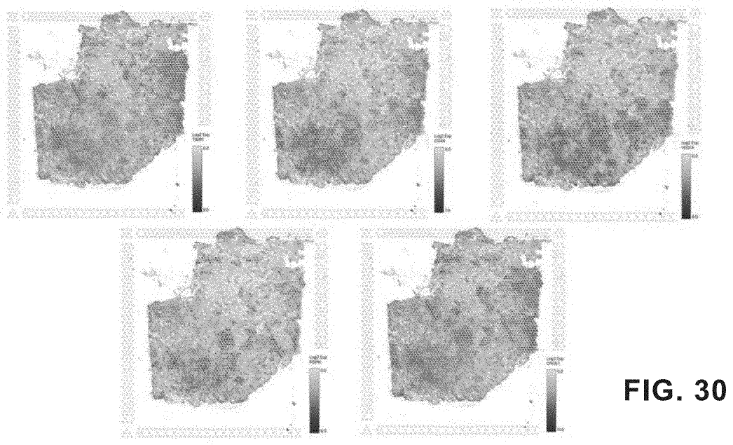

Spatial analysis of glioblastoma samples demonstrated regional distribution of overexpressed genes in glioblastoma (FIG. 30).

### Example 6: Comparison Between Healthy and Glioblastoma Samples

Spatial analysis was performed on healthy (nonspecific &temporal) and glioblastoma samples from different patients. Four tissue sections from 1 glioblastoma sample and a total of nine tissue sections from two control samples (4 sections from one control sample and 5 sections from the second control sample) were used to create whole transcriptome sequencing libraries. Whole transcriptome sequencing and analysis showed that genes were differentially expressed, for example, the genes shown in Table 8.

TABLE 8
top overexpressed genes in glioblastoma relative
to normal, whole transcriptome results.

| | | | Glioblastoma | |
|---|---|---|---|---|
| | | Glioblastoma | Log2 Fold | Glioblastoma |
| FeatureID | FeatureName | Average | Change | P-Value |
| ENSG00000170290 | SLN | 1.79572485 | 7.11452788 | 0 |
| ENSG00000102359 | SRPX2 | 1.2604062 | 5.6423875 | 1.14E−255 |
| ENSG00000170439 | METTL7B | 2.04773939 | 5.3733812 | 7.59E−237 |
| ENSG00000133110 | POSTN | 2.83567896 | 5.23122146 | 1.81E−227 |
| ENSG00000166741 | NNMT | 1.19618979 | 5.23563833 | 1.62E−225 |
| ENSG00000157150 | TIMP4 | 1.08558475 | 5.10925969 | 1.21E−217 |
| ENSG00000196136 | SERPINA3 | 5.4768289 | 4.72097273 | 4.10E−193 |
| ENSG00000162873 | KLHDC8A | 1.13643032 | 4.71148824 | 1.94E−192 |
| ENSG00000132688 | NES | 3.26063514 | 4.61622082 | 2.71E−187 |
| ENSG00000134531 | EMP1 | 3.15206151 | 4.42972224 | 1.66E−174 |
| ENSG00000181104 | F2R | 1.23857625 | 4.41003053 | 1.46E−172 |
| ENSG00000229807 | XIST | 1.61098898 | 4.39884917 | 2.13E−171 |
| ENSG00000168542 | COL3A1 | 1.17496623 | 4.41375537 | 2.09E−166 |
| ENSG00000113140 | SPARC | 19.3375981 | 4.20699279 | 4.10E−161 |
| ENSG00000164692 | COL1A2 | 1.58994734 | 4.11304809 | 4.54E−150 |
| ENSG00000187498 | COL4A1 | 1.45266127 | 4.1109279 | 9.21E−149 |
| ENSG00000074410 | CA12 | 1.35700368 | 4.03323633 | 9.49E−148 |
| ENSG00000182718 | ANXA2 | 2.17859775 | 3.9971458 | 1.41E−146 |
| ENSG00000018408 | WWTR1 | 2.44183045 | 3.99077399 | 2.26E−146 |
| ENSG00000102265 | TIMP1 | 15.495953 | 3.95925361 | 1.73E−144 |
| ENSG00000179431 | FJX1 | 1.95811535 | 3.85754333 | 8.75E−138 |
| ENSG00000144810 | COL8A1 | 1.74218086 | 3.77807497 | 3.24E−132 |
| ENSG00000159403 | C1R | 1.50108129 | 3.7627829 | 7.79E−132 |
| ENSG00000163565 | IFI16 | 1.30015488 | 3.66606153 | 8.00E−126 |
| ENSG00000182326 | C1S | 1.2276916 | 3.60670229 | 3.59E−122 |
| ENSG00000115414 | FN1 | 2.86026797 | 3.60549473 | 2.98E−120 |
| ENSG00000172037 | LAMB2 | 1.48695247 | 3.54299336 | 8.10E−119 |
| ENSG00000134668 | SPOCD1 | 1.30752248 | 3.49230713 | 2.04E−114 |
| ENSG00000131435 | PDLIM4 | 1.15774484 | 3.38491192 | 4.87E−109 |
| ENSG00000110492 | MDK | 1.28423721 | 3.18784985 | 1.52E−97 |
| ENSG00000185201 | IFITM2 | 2.2629159 | 3.16213754 | 9.48E−97 |
| ENSG00000245532 | NEAT1 | 5.94474762 | 3.11898334 | 1.16E−94 |
| ENSG00000149131 | SERPING1 | 2.81002878 | 3.09823979 | 1.84E−93 |
| ENSG00000142156 | COL6A1 | 2.1866627 | 3.06636705 | 1.22E−91 |
| ENSG00000164434 | FABP7 | 4.107273 | 3.05359799 | 4.48E−91 |
| ENSG00000204287 | HLA-DRA | 1.4910759 | 3.00103364 | 5.91E−88 |
| ENSG00000101955 | SRPX | 1.19876693 | 3.02158944 | 7.46E−88 |
| ENSG00000175899 | A2M | 6.9004139 | 2.97943542 | 2.34E−87 |
| ENSG00000138448 | ITGAV | 2.72458882 | 2.96132092 | 2.74E−86 |
| ENSG00000105855 | ITGB8 | 2.14930925 | 2.95931109 | 3.68E−86 |
| ENSG00000118785 | SPP1 | 19.7822315 | 2.90867342 | 1.84E−83 |
| ENSG00000026025 | VIM | 28.2939982 | 2.8893192 | 1.60E−82 |
| ENSG00000162493 | PDPN | 1.15159001 | 2.88555631 | 3.51E−81 |
| ENSG00000110799 | VWF | 1.01976142 | 2.89038134 | 7.21E−81 |
| ENSG00000115380 | EFEMP1 | 1.93540615 | 2.86314337 | 7.67E−81 |
| ENSG00000164111 | ANXA5 | 2.8731537 | 2.85432725 | 1.16E−80 |
| ENSG00000026508 | CD44 | 2.20100376 | 2.85568845 | 4.17E−80 |
| ENSG00000163191 | S100A11 | 1.72347381 | 2.83125421 | 1.77E−78 |
| ENSG00000060982 | BCAT1 | 1.20486112 | 2.80431383 | 1.26E−77 |
| ENSG00000080493 | SLC4A4 | 2.76451942 | 2.78461039 | 5.17E−77 |
| ENSG00000181722 | ZBTB20 | 1.32422845 | 2.76771188 | 7.81E−76 |
| ENSG00000163584 | RPL22L1 | 1.03422376 | 2.77109136 | 1.29E−75 |
| ENSG00000106278 | PTPRZ1 | 5.56918168 | 2.75134542 | 1.50E−75 |
| ENSG00000162430 | SELENON | 1.45572353 | 2.7580052 | 2.84E−75 |
| ENSG00000163453 | IGFBP7 | 8.54184352 | 2.73694606 | 1.35E−74 |
| ENSG00000114115 | RBP1 | 1.12181639 | 2.72610485 | 1.38E−73 |
| ENSG00000062716 | VMP1 | 2.04034146 | 2.713888 | 4.76E−73 |
| ENSG00000168615 | ADAM9 | 1.09665132 | 2.66972765 | 7.30E−71 |
| ENSG00000206503 | HLA-A | 6.85047792 | 2.64688475 | 2.91E−70 |
| ENSG00000135404 | CD63 | 8.52225721 | 2.64335333 | 3.81E−70 |
| ENSG00000011465 | DCN | 1.02033749 | 2.67132502 | 6.26E−70 |
| ENSG00000234745 | HLA-B | 5.63476246 | 2.61600871 | 1.26E−68 |
| ENSG00000105835 | NAMPT | 1.69172944 | 2.60619255 | 1.41E−67 |
| ENSG00000112715 | VEGFA | 4.77830112 | 2.5993549 | 3.89E−67 |
| ENSG00000105894 | PTN | 7.42108831 | 2.57308207 | 1.03E−66 |
| ENSG00000158710 | TAGLN2 | 1.20792338 | 2.55843037 | 3.45E−65 |
| ENSG00000249992 | TMEM158 | 1.07457883 | 2.52534622 | 8.88E−64 |
| ENSG00000019582 | CD74 | 3.11288889 | 2.48616331 | 2.79E−62 |
| ENSG00000125148 | MT2A | 44.2981341 | 2.47099387 | 1.27E−61 |
| ENSG00000150093 | ITGB1 | 1.58952287 | 2.46958778 | 2.84E−61 |
| ENSG00000122786 | CALD1 | 1.52651924 | 2.42166646 | 4.16E−59 |
| ENSG00000108679 | LGALS3BP | 1.34399668 | 2.3881849 | 1.67E−57 |
| ENSG00000144136 | SLC20A1 | 1.8275905 | 2.38033065 | 2.91E−57 |
| ENSG00000177697 | CD151 | 1.03252587 | 2.38222765 | 8.25E−57 |
| ENSG00000142089 | IFITM3 | 3.19726768 | 2.34126085 | 1.12E−55 |
| ENSG00000166710 | B2M | 10.7930561 | 2.33248744 | 1.86E−55 |
| ENSG00000113594 | LIFR | 1.33101999 | 2.31802452 | 1.75E−54 |
| ENSG00000118705 | RPN2 | 1.42798131 | 2.3153264 | 2.15E−54 |
| ENSG00000145824 | CXCL14 | 3.19726768 | 2.29573873 | 1.24E−53 |
| ENSG00000002586 | CD99 | 2.16449925 | 2.28033431 | 6.36E−53 |
| ENSG00000139289 | PHLDA1 | 1.10626256 | 2.28238255 | 1.17E−52 |
| ENSG00000136158 | SPRY2 | 1.38016768 | 2.26381838 | 4.45E−52 |
| ENSG00000111341 | MGP | 2.84113645 | 2.27064135 | 9.78E−52 |
| ENSG00000148926 | ADM | 1.66798938 | 2.26777313 | 2.18E−51 |
| ENSG00000115461 | IGFBP5 | 3.12914007 | 2.24386561 | 3.44E−51 |
| ENSG00000135046 | ANXA1 | 1.84511509 | 2.25296807 | 3.65E−51 |
| ENSG00000138434 | ITPRID2 | 1.12742547 | 2.24158614 | 4.33E−51 |
| ENSG00000106236 | NPTX2 | 1.15868474 | 2.2153727 | 7.05E−50 |
| ENSG00000185222 | TCEAL9 | 1.24227522 | 2.20407075 | 1.87E−49 |
| ENSG00000103187 | COTL1 | 1.08397783 | 2.18330218 | 1.51E−48 |
| ENSG00000204525 | HLA-C | 2.59342726 | 2.17883952 | 1.53E−48 |
| ENSG00000102024 | PLS3 | 1.18203064 | 2.17979207 | 2.07E−48 |
| ENSG00000143870 | PDIA6 | 1.40375614 | 2.17324256 | 3.50E−48 |
| ENSG00000100644 | HIF1A | 1.42194776 | 2.14266248 | 5.76E−47 |
| ENSG00000185624 | P4HB | 2.12908623 | 2.10785913 | 1.53E−45 |
| ENSG00000204580 | DDR1 | 1.81079357 | 2.08621227 | 1.36E−44 |
| ENSG00000166340 | TPP1 | 1.85684868 | 2.07020271 | 4.68E−44 |
| ENSG00000124225 | PMEPA1 | 1.02403645 | 2.05406382 | 3.00E−43 |
| ENSG00000183255 | PTTG1IP | 1.42852706 | 2.01531059 | 9.05E−42 |
| ENSG00000089157 | RPLP0 | 5.56678645 | 2.00470346 | 1.41E−41 |

In addition, the whole transcriptome sequencing library was selectively enriched for targeted sequencing using hybrid capture pulldown of a panel of human neuroscience-related transcripts. Targeted sequencing and analysis showed that genes were differentially expressed, for example, the genes shown in Table 9.

TABLE 9
top overexpressed genes in glioblastoma relative
to normal, targeted sequencing results.

| | | | Glioblastoma | |
|---|---|---|---|---|
| | | Glioblastoma | Log2 Fold | Glioblastoma |
| FeatureID | FeatureName | Average | Change | P-Value |
| ENSG00000132688 | NES | 3.43009676 | 4.6310742 | 4.84E−205 |
| ENSG00000187498 | COL4A1 | 1.28661071 | 4.22722676 | 3.65E−173 |
| ENSG00000172037 | LAMB2 | 1.72165588 | 3.78208577 | 7.19E−148 |
| ENSG00000113140 | SPARC | 9.16340877 | 3.6887985 | 1.17E−142 |
| ENSG00000115414 | FN1 | 2.13504587 | 3.59808635 | 3.61E−134 |
| ENSG00000067182 | TNFRSF1A | 1.0292648 | 3.32218081 | 1.52E−118 |
| ENSG00000204287 | HLA-DRA | 1.43134179 | 3.25290006 | 1.07E−114 |
| ENSG00000144908 | ALDH1L1 | 1.31319923 | 2.96682169 | 4.31E−98 |
| ENSG00000026508 | CD44 | 1.93373 | 2.89811978 | 5.48E−94 |
| ENSG00000196924 | FLNA | 1.75464387 | 2.71813036 | 4.13E−84 |
| ENSG00000105835 | NAMPT | 1.37749113 | 2.71648475 | 7.64E−84 |
| ENSG00000112715 | VEGFA | 3.3302016 | 2.68422099 | 5.11E−82 |
| ENSG00000125730 | C3 | 1.13187427 | 2.65710698 | 1.03E−80 |
| ENSG00000206503 | HLA-A | 5.27657205 | 2.57580858 | 6.24E−77 |
| ENSG00000030582 | GRN | 1.03818047 | 2.56578626 | 6.34E−76 |
| ENSG00000234745 | HLA-B | 3.65550476 | 2.52262648 | 4.97E−74 |
| ENSG00000118785 | SPP1 | 8.5402033 | 2.50180907 | 6.74E−73 |
| ENSG00000166340 | TPP1 | 2.24066687 | 2.4417344 | 8.04E−70 |
| ENSG00000115457 | IGFBP2 | 3.52339431 | 2.37417656 | 4.57E−66 |
| ENSG00000148926 | ADM | 1.65249008 | 2.36926752 | 3.29E−65 |
| ENSG00000100644 | HIF1A | 1.3881305 | 2.24350791 | 8.27E−60 |
| ENSG00000106366 | SERPINE1 | 1.45915869 | 2.10910808 | 7.13E−53 |
| ENSG00000170558 | CDH2 | 1.07200058 | 2.07406104 | 7.09E−52 |
| ENSG00000125398 | SOX9 | 1.08987155 | 1.82351282 | 5.27E−41 |
| ENSG00000119655 | NPC2 | 1.10399794 | 1.8151279 | 1.20E−40 |
| ENSG00000181449 | SOX2 | 1.6308349 | 1.7687618 | 6.86E−39 |
| ENSG00000205336 | ADGRG1 | 1.43494768 | 1.74713688 | 4.81E−38 |
| ENSG00000131981 | LGALS3 | 2.89394787 | 1.70038942 | 4.88E−36 |
| ENSG00000079215 | SLC1A3 | 4.93541881 | 1.53182532 | 5.49E−30 |
| ENSG00000108518 | PFN1 | 2.23670434 | 1.52433325 | 1.16E−29 |
| ENSG00000114353 | GNAI2 | 1.5285028 | 1.51892911 | 1.96E−29 |
| ENSG00000130203 | APOE | 13.9945538 | 1.47722043 | 4.82E−28 |
| ENSG00000160307 | S100B | 5.17523057 | 1.46571086 | 1.21E−27 |
| ENSG00000161011 | SQSTM1 | 1.8469706 | 1.40154042 | 1.96E−25 |
| ENSG00000112096 | SOD2 | 1.85770904 | 1.34069361 | 3.61E−23 |
| ENSG00000131095 | GFAP | 12.2349766 | 1.33087211 | 3.62E−23 |
| ENSG00000117984 | CTSD | 2.96440149 | 1.30991243 | 1.72E−22 |
| ENSG00000148180 | GSN | 1.74864065 | 1.24216165 | 2.16E−20 |
| ENSG00000113712 | CSNK1A1 | 1.07023726 | 1.09957174 | 2.48E−16 |
| ENSG00000102144 | PGK1 | 2.18243762 | 1.09112437 | 3.78E−16 |
| ENSG00000067560 | RHOA | 1.80364044 | 1.07206323 | 1.19E−15 |
| ENSG00000137710 | RDX | 1.12412755 | 1.06725974 | 1.77E−15 |
| ENSG00000184009 | ACTG1 | 7.72741102 | 1.03244178 | 1.15E−14 |
| ENSG00000122566 | HNRNPA2B1 | 2.36516927 | 1.03025827 | 1.33E−14 |
| ENSG00000123384 | LRP1 | 1.41273775 | 0.9924383 | 1.24E−13 |
| ENSG00000084207 | GSTP1 | 1.21027275 | 0.9897912 | 1.48E−13 |
| ENSG00000106211 | HSPB1 | 1.84550447 | 0.95859759 | 8.04E−13 |
| ENSG00000120885 | CLU | 15.3904707 | 0.94628642 | 1.40E−12 |
| ENSG00000133048 | CHI3L1 | 3.4428957 | 0.94483501 | 2.95E−12 |
| ENSG00000185896 | LAMP1 | 1.06946457 | 0.93909228 | 2.32E−12 |
| ENSG00000177700 | POLR2L | 1.23698014 | 0.83317238 | 4.86E−10 |
| ENSG00000168036 | CTNNB1 | 1.31092078 | 0.8153585 | 1.13E−09 |
| ENSG00000128272 | ATF4 | 1.18742882 | 0.76698972 | 1.07E−08 |
| ENSG00000152661 | GJA1 | 1.55762733 | 0.623434 | 3.24E−06 |
| ENSG00000136156 | ITM2B | 4.66743352 | 0.52479654 | 8.90E−05 |
| ENSG00000112531 | QKI | 1.93178836 | 0.4993827 | 0.00019628 |
| ENSG00000156508 | EEF1A1 | 2.69023467 | 0.37810806 | 0.00493817 |
| ENSG00000189403 | HMGB1 | 2.33101234 | 0.28006584 | 0.03811429 |
| ENSG00000189058 | APOD | 1.3076517 | 0.24009539 | 0.07689109 |
| ENSG00000240972 | MIF | 3.70820628 | 0.11748718 | 0.39079095 |
| ENSG00000018625 | ATP1A2 | 1.48800584 | 0.09612068 | 0.48612996 |
| ENSG00000136238 | RAC1 | 1.37061616 | 0.05141084 | 0.71150614 |
| ENSG00000115053 | NCL | 1.38064134 | 0.04106448 | 0.76710185 |

Several overexpressed genes appeared in the whole transcriptome and targeted sequencing results. See Table 10 below.

TABLE 10
overexpressed genes in glioblastoma relative to normal tissue, as shown
in both whole transcriptome and targeted sequencing approaches.
Overlapping Genes

ADM
CD44
FN1
HLA-A
HLA-B
HLA-DRA
LAMB2
NAMPT
NES
SPARC
SPP1
VEGFA

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of determining abundance of two or more analytes in a subject having glioblastoma, comprising
determining the abundance of the two or more analytes selected from the group consisting of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, and MT-ND5 and byproducts, precursors and degradation products thereof, in a biological sample obtained from a subject.

2. The method of claim 1, wherein the two or more analytes further comprise
CD44, POSTN, NES, TERT, UMOD, SGK1, GPR37L1, ISG15, or RGS5, or a byproduct or precursor or degradation product thereof, in the biological sample from a subject.

3. The method of claim 1, wherein the two or more analytes further comprise
SPOCD1, DDK1, TNC, GBE1, SMIM3, CLIC1, MT1X, or CYR61, or a byproduct or precursor or degradation product thereof, in the biological sample from a subject.

4. The method of claim 1, wherein the method further comprises:
(a) determining the abundance of SPOCD1, DDK1, TNC, GBE1, SMIM3, CLIC1, MT1X, and CYR61, or a byproduct or precursor or degradation product thereof, in a biological sample from a subject; and
(b) administering a treatment for glioblastoma to a subject having an elevated abundance of SPOCD1, DDK1, TNC, GBE1, SMIM3, CLIC1, MT1X, and CYR61 or a byproduct or precursor or degradation product thereof, in the biological sample as compared to a reference level.

5. A method of diagnosing a subject as having glioblastoma, wherein the method comprises:
(a) determining elevated abundance of ionized calcium-binding adaptor molecule 1 (IBA1);
(b) determining the abundance of two or more analytes selected from the group consisting of DKK1, CHI3L1, HS2ST1, EGR1, TCIM, PLIN2, APOC1, FOS, MGP, SPP1, RPL17, TNC, IFITM3, MT2A, TMSB4X, TMSB10, PDPN, COX6C, VIM, CLIC1, IFITM2, TCEAL9, RPL12, TAGLN, NAMPT, HBA2, HBB, HBA1, COL1A2, MALAT1, RBM25, SLC25A37, NKTR, LUC7L3, ATP1A2, PNISR, MEG3, IFI44L, FAM133B, PNN, PLEKHA4, PTMS, BDP1, MTRNR2L12, SREK1, ARGLU1, XAF1, MTRNR2L8, SRRM2, and COL4A1 and byproducts, precursors, and degradation products thereof, in areas of a biological sample from a subject having elevated IBA1 compared to a reference level of abundance; and
(c) identifying a subject having:
(i) elevated abundance of the two or more analytes DKK1, CHI3L1, HS2ST1, EGR1, TCIM, PLIN2, APOC1, FOS, MGP, SPP1, RPL17, TNC, IFITM3, MT2A, TMSB4X, TMSB10, PDPN, COX6C, VIM, CLIC1, IFITM2, TCEAL9, RPL12, TAGLN, and NAMPT and byproducts, precursors, and degradation products thereof, in the areas as compared to the reference level of abundance, or
(ii) decreased abundance of the two or more analytes HBA2, HBB, HBA1, COL1A2, MALAT1, RBM25, SLC25A37, NKTR, LUC7L3, ATP1A2, PNISR MEG3, IFI44L, FAM133B, PNN, PLEKHA4, PTMS, BDP1, MTRNR2L12, SREK1, ARGLU1, XAF1, MTRNR2L8, SRRM2, and COL4A1 and byproducts, precursors, and degradation products thereof, in the areas as compared to the reference level of abundance, as having glioblastoma.

6. The method of claim 5, wherein the two or more analytes are selected from the group consisting of DKK1, HS2ST1, EGR1, TCIM, FOS, RPL17, TNC, IFITM3, TMSB4X, TMSB10, COX6C, CLIC1, TCEAL9, and RPL12 and byproducts, precursors, and degradation products thereof.

7. The method of claim 1, wherein the method further comprises:
(a) determining the abundance of two or more analytes selected from the group consisting of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, CD44, POSTN, NES, TERT, UMOD, SGK1, GPR37L1, ISG15, RGS5, SPOCD1, DDK1, TNC, GBE1, SMIM3, CLIC1, MT1 X, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, and MT-ND5, and byproducts, precursors, and degradation products thereof, in a biological sample from a subject;
(b) administering a treatment for glioblastoma to the subject having
(i) an elevated abundance of two or more analytes selected from the group consisting of COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, CD44, POSTN, NES, TERT, UMOD, SGK1, GPR37L1, ISG15, RGS5, SPOCD1, DDK1, TNC, GBE1, SMIM3, CLIC1, MT1X, and byproducts, precursors, and degradation products thereof, in a biological sample from a subject; or
(ii) a decreased abundance of two or more analytes selected from the group consisting of GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, and MT-ND5, and byproducts, precursors and degradation products thereof, in the biological sample as compared to a reference level of abundance.

8. The method of claim 7, further comprising:
(c) determining an abundance of two or more analytes selected from the group consisting of NAPB, BASP1, RUNDC3A, NEFM, RAB3A, GNG3, KIF1A, ATP1A3, CNTN1, CELF4, SYN2, TUBB4A, and GRIN1, and byproducts precursors and degradation products thereof, in a biological sample from a subject; and
(d) administering a treatment for glioblastoma to the subject having decreased abundance of the two or more analytes of step (c) in the biological sample as compared to a reference level of abundance.

9-10. (canceled)

11. The method of claim 5, wherein the two or more analytes are selected from the group consisting of HBA2, HBB, HBA1, MALAT1, RBM25, SLC25A37, NKTR, LUC7L3, PNISR, MEG3, IFI44L, FAM133B, PNN, PLEKHA4, PTMS, BDP1, MTRNR2L12, SREK1, ARGLU1, XAF1, MTRNR2L8, and SRRM2 and byproducts, precursors and degradation products thereof.

12. The method of claim 1, wherein the method further comprises confirming a diagnosis of glioblastoma in the subject by obtaining an image of the subject's brain or performing neurological testing on the subject.

13. The method of any one of claim 1, wherein the biological sample from the subject comprises more than one biological sample from the subject from a plurality of time points and determining the abundance of the two or more analytes in the two or more biological samples from the plurality of time points from the subject.

14-28. (canceled)

29. The method of claim 1, wherein the biological sample comprises brain tissue or cerebrospinal fluid.

30-71. (canceled)

72. The method of claim 1, wherein the two or more analytes are mRNA molecules.

73. The method of claim 72, wherein the determining step comprises:
(a) contacting the biological sample with a substrate comprising a plurality of attached capture probes, wherein a capture probe of the plurality of attached capture probes comprises (i) a spatial barcode and (ii) a capture domain that binds to a sequence present in the analyte;
(b) hybridizing the two or more analytes to the capture domain;
(c) extending a 3′ end of the capture probe using the analyte that is bound to the capture domain as a template to generate an extended capture probe;
(d) amplifying the extended capture probe; and
(e) determining (i) all or a portion of the sequence of the spatial barcode or the complement thereof, and (ii) all or a portion of the sequence of the analyte from the biological sample; and using the determined sequences of (i) and (ii) to identify the location of the analyte in the biological sample, thereby determining the abundance and location of the two or more analytes.

74-77. (canceled)

78. The method of claim 1, wherein the two or more analytes are proteins.

79. The method of claim 78, wherein the determining step comprises determining the abundance and location of the two or more analytes, the method comprising:
(a) attaching the biological sample with a plurality of analyte capture agents, wherein an analyte capture agent of the plurality of analyte capture agents comprises:
(i) an analyte binding moiety that binds to the two or more analytes
(ii) an analyte binding moiety barcode that uniquely identifies an interaction between the two or more analytes and the analyte binding moiety; and
(iii) an analyte capture sequence, wherein the analyte capture sequence binds to a capture domain;
(b) contacting the biological sample with a substrate, wherein the substrate comprises a plurality of capture probes, wherein a capture probe of the plurality of capture probes comprises (i) the capture domain and (ii) a spatial barcode;
(c) hybridizing the two or more analytes to the capture probe; and
(d) determining (i) all or a part of a sequence corresponding to the analyte binding moiety barcode, and (ii) all or a part of a sequence corresponding to the spatial barcode, or a complement thereof, and using the determined sequence of (i) and (ii) to identify the abundance and spatial location of the two or more analytes in the biological sample.

80-104. (canceled)

105. The method of claim 1, further comprising administering a treatment for glioblastoma to the subject, wherein the treatment comprises surgery, chemotherapeutic agents, growth inhibitory agents, cytotoxic agents, agents used in radiation therapy, anti-angiogenesis agents, cancer immunotherapeutic agents, apoptotic agents, anti-tubulin agents, or a combination thereof.

106-107. (canceled)

108. A kit comprising:
an antibody that binds specifically to COL1A1, COL3A1, COL8A1, WEE1, CHI3L1, MGP, SRPX, SERPINE1, COL1A2, TIMP1, ANXA1, COL6A2, CAV1, PLIN2, CD44, APOC1, IGFBP2, PDPN, VIM, LGALS3, VEGFA, IGFBP5, CTGF, EMP1, EMP3, IGFBP3, A2M, ANXA2, FLNA, IFGBP7, S100A11, ADM, FN1, SERPING1, MT2A, S100A10, SPARC, ITGB1, SLC5A3, FABP7, YBX3, IFITM2, TAGLN2, COL6A1, HLA-A, LGALS3BP, ANXA5, APOE, GADD45A, TPM4, SPP1, GABRA1, CCK, SLC17A7, CHGA, STMN2, CALY, EEF1A2, CABP1, NRGN, SNAP25, ATP2B2, SYN1, NECAB1, MBP, PHYHIP, BASP, CPLX1, VSNL1, TAGLN3, ENC1, FBXL16, CHN1, KIF5A, PLP1, OLFM1, SNCB, STXBP1, ATP1B1, DNM1, SERPINI1, PRKAR1B, MEF2C, MTURN, NSF, SYT1, MAP2, MT-ATP8, MAP1A, UCHL1, FAIM2, STMN1, APLP1, NCDN, STMN3, MT-ND4L, BEX1, MT-ND2, PPP3CA, CPLX2, ST8SIA3, GABRG2, KCNC2, MT-ND5, CD44, POSTN, NES, TERT, UMOD, SGK1, GPR37L1, ISG15, RGS5, SPOCD1, DDK1, TNC, GBE1, SMIM3, CLIC1, MTX, CYR61, NAPB, BASP1, RUNDC3A, NEFM, RAB3A, GNG3, KIF1A, ATP1A3, CNTN1, CELF4, SYN2, TUBB4A, GRIN1, DKK1, HS2ST1, EGR1, TCIM, FOS, RPL17, TNC, IFITM3, TMSB4X, TMSB10, COX6C, CLIC1, TCEAL9, RPL12, HBA2, HBB, HBA1, MALAT1, RBM25, SLC25A37, NKTR, LUC7L3, PNISR, MEG3, IFI44L, FAM133B, PNN, PLEKHA4, PTMS, BDP1, MTRNR2L12, SREK1, ARGLU1, XAF1, MTRNR2L8, SRRM2, or a byproduct or precursor or degradation product thereof, or any combination thereof, and
instructions for performing the method of claim 73.
